(19) United States
(12) Patent Application Publication
(10) Pub. No.: US 2023/0338473 A1
(43) Pub. Date: Oct. 26, 2023

(54) ANTAGONISTIC PEPTIDE TARGETING IL-2, IL-9, AND IL-15 SIGNALING FOR THE TREATMENT OF CYTOKINERELEASE SYNDROME AND CYTOKINE STORM ASSOCIATED DISORDERS

(71) Applicant: Bioniz, LLC, Irvine, CA (US)

(72) Inventors: Yutaka Tagaya, Rockville, MD (US); Nazli Azimi, San Juan Capistrano, CA (US)

(21) Appl. No.: 18/002,732

(22) PCT Filed: Jun. 22, 2021
(86) PCT No.: PCT/US2021/038512

§ 371 Date: Dec. 21, 2022

Related U.S. Application Data
(60) Provisional application No. 63/043,636, filed on Jun. 24, 2020.

(51) Int. Cl. C07K 14/54; A61K 38/20; C07K 14/55; A61P 35/00

(52) U.S. Cl. CPC A61K 38/2013; A61K 38/2086; A61P 35/00; C07K 14/5443; C07K 14/55

(57) ABSTRACT

The yc-family Interleukin-2 (IL-2), Interleukin-9 (IL-9), and Interleukin-15 (IL-15) cytokines are associated with important human diseases, such as cytokine-release syndrome and cytokine storm associated disorders. Compositions, methods, and kits to modulate signaling by at least one IL-2, IL-9, or IL-15 γc-cytokine family members for inhibiting, ameliorating, reducing a severity of, treating, delaying the onset of, or preventing at least one cytokine storm related disorder are described.

### CROSS-REFERENCE TO RELATED APPLICATIONS

[0000A] This application claims the benefit of U.S. Provisional Application 63/043,636 filed on Jun. 24, 2020, which is hereby incorporated by reference in its entirety.

### REFERENCE TO ELECTRONIC SEQUENCE LISTING

[0000B] The present application is being filed along with an Electronic Sequence Listing. The Electronic Sequence Listing is provided as a file entitled BION014WOSEQLIST.txt which is 15,648 bytes in size, created on Jun. 21, 2021. The information in the Electronic Sequence Listing is incorporated herein by reference in its entirety.

### FIELD

The present embodiments relate to inhibiting, ameliorating, reducing a severity of, treating, delaying the onset of, or preventing immune-mediated diseases such as cytokine-release syndrome (also known as cytokine storm), and cytokine storm associated disorders using a γc-cytokine antagonist peptide, or a derivative thereof, by modulating the signaling by at least one of IL-2, IL-9, and IL-15 γc-cytokine family members.

### BACKGROUND

Cytokines are a diverse group of soluble factors that mediate various cell functions, such as, growth, functional differentiation, and promotion or prevention of programmed cell death (apoptotic cell death). Cytokines, unlike hormones, are not produced by specialized glandular tissues, but can be produced by a wide variety of cell types, such as epithelial, stromal or immune cells.

The γc-family cytokines are a group of mammalian cytokines that are mainly produced by epithelial, stromal and immune cells and control the normal and pathological activation of a diverse array of lymphocytes. These cytokines are critically required for the early development of T cells in the thymus as well as their homeostasis in the periphery.

### SUMMARY

Some embodiments, disclosed herein pertain to therapeutic compounds, compositions comprising the same, methods of using the same for the treatment of disease states, and/or methods of manufacturing the same.

Some embodiments pertain to a composition. In some embodiments, the composition comprises a therapeutic compound that is a γc-cytokine antagonist peptide, or a derivative thereof, in an amount sufficient to inhibit signaling by at least one of IL-2, IL-9, and IL-15 γc-cytokine family members, thereby inhibiting, ameliorating, reducing a severity of, treating, delaying the onset of, or preventing at least one cytokine storm related disorder. In some embodiments, the composition further comprises a pharmaceutically acceptable carrier.

In some embodiments of the composition, the at least one cytokine storm related disorder (e.g., the at least one cytokine storm associated disorder) is selected from the group consisting of cytokine release syndrome, cytokine storm, multiple organ dysfunction syndrome, systemic inflammatory response syndrome, sepsis, septic shock, graft-versus-host disease, haploidentical donor transplantation, sarcoidosis, hemophagocytic lymphohistiocytosis, vascular leak syndrome, systemic capillary leak syndrome, Stevens-Johnson syndrome, toxic epidermal necrolysis, asthmatic allergic lung inflammation, rhinosinusitis, viral infection, coronavirus infection, multi-system inflammatory syndrome in children (MIS-C) associated with coronavirus disease (e.g., COVID, COVID-19, etc.), viral hemorrhagic fever, influenza viral infection, hantaviral infection, Epstein-Barr viral infection, HIV/HCV coinfection liver fibrosis, fungal infection, pulmonary Aspergillosis, bacterial infection, toxic shock syndrome, lyme neuroborreliosis, lyme disease, autoimmune disease, juvenile idiopathic arthritis, Still’s disease, macrophage activation syndrome, Sjögren’s syndrome, systemic sclerosis, inflammatory myopathies, systemic vasculitides, giant cell arteritis, Horton disease, cranial arteritis, temporal arteritis, T-cell based immunotherapy induced cytokine storm, chimeric antigen receptor T-cell therapy induced cytokine storm, immune effector cell-associated neurotoxicity syndrome, T-cell bispecific antibody therapy induced cytokine storm, pulmonary infiltrate, adult respiratory distress syndrome, interstitial lung disease, pneumonia, community acquired pneumonia, acute interstitial pneumonia, and/or combinations of any of the foregoing.

In some embodiments of the composition, the therapeutic compound is at least one of a γc cytokine antagonist peptide, a γc cytokine antagonist peptide derivative, and/or a combination thereof.

In some embodiments of the composition, the γc cytokine antagonist peptide comprises a partial sequence of a γc-box D-helix region of IL-2 and IL-15 γc-cytokine family members.

In some embodiments of the composition, the partial sequence comprises consecutive blocks of at least 5 amino acids of the γc-box D-helix region of each IL-2 and IL-15 γc-cytokine family members.

In some embodiments of the composition, the partial sequence comprises consecutive blocks of 1-10 amino acids of the γc-box D-helix region of each IL-2 and IL-15 γc-cytokine family members.

In some embodiments of the composition, the γc cytokine antagonist peptide comprises 11 to 50 amino acids.

In some embodiments of the composition, the γc cytokine antagonist peptide further comprises a conjugate at the N-termini, C-termini, side residues, or a combination thereof.

In some embodiments of the composition, the conjugate comprises one or more additional moieties selected from the group consisting of bovine serum albumin (BSA), albumin, Keyhole Limpet Hemocyanin (KLH), Fc region of IgG, a biological protein that functions as scaffold, an antibody against a cell-specific antigen, a receptor, a ligand, a metal ion, and Poly Ethylene Glycol (PEG).

In some embodiments of the composition, the γc cytokine antagonist peptide further comprises a signal peptide.

In some embodiments of the composition, the γc cytokine antagonist peptide comprises a sequence of SEQ ID NO: 1 (BNZ-γ)

In some embodiments of the composition, the γc cytokine antagonist peptide and the γc antagonist peptide derivative have similar physico-chemical properties but distinct IL-2, IL-9, and IL-15 biological activities.

In some embodiments of the composition, the γc cytokine antagonist peptide derivative shares at least about 60% identity with a peptide of SEQ ID NO: 1.

In some embodiments of the composition, the γc cytokine antagonist peptide derivative shares at least about 90% identity with a peptide of SEQ ID NO: 1.

In some embodiments of the composition, the γc cytokine antagonist peptide derivative shares at least about 95% identity with a peptide of SEQ ID NO: 1.

In some embodiments of the composition, the pharmaceutically acceptable carrier is formulated for topical, oral, and/or parenteral delivery.

In some embodiments of the composition, the pharmaceutically acceptable carrier is formulated for topical delivery.

In some embodiments of the composition, the pharmaceutically acceptable carrier is formulated for oral delivery.

In some embodiments of the composition, the pharmaceutically acceptable carrier is formulated for parenteral delivery.

In some embodiments, a method of inhibiting, ameliorating, reducing a severity of, treating, delaying the onset of, or preventing at least one cytokine storm related disorder comprises administering one or more of the compositions provided herein to a subject in need thereof, thereby inhibiting, ameliorating, reducing a severity of, treating, delaying the onset of, or preventing the at least one cytokine storm related disorder.

In some embodiments of the method of inhibiting, ameliorating, reducing a severity of, treating, delaying the onset of, or preventing at least one cytokine storm related disorder, the at least one cytokine storm related disorder is selected from the group consisting of cytokine release syndrome, cytokine storm, multiple organ dysfunction syndrome, systemic inflammatory response syndrome, sepsis, septic shock, graft-versus-host disease, haploidentical donor transplantation, sarcoidosis, hemophagocytic lymphohistiocytosis, vascular leak syndrome, systemic capillary leak syndrome, Stevens-Johnson syndrome, toxic epidermal necrolysis, asthmatic allergic lung inflammation, rhinosinusitis, viral infection, coronavirus infection, multi-system inflammatory syndrome in children (MIS-C) associated with COVID-19 (or a different coronavirus disease), viral hemorrhagic fever, influenza viral infection, hantaviral infection, Epstein-Barr viral infection, HIV/HCV coinfection liver fibrosis, fungal infection, pulmonary Aspergillosis, bacterial infection, toxic shock syndrome, lyme neuroborreliosis, lyme disease, autoimmune disease, juvenile idiopathic arthritis, Still’s disease, macrophage activation syndrome, Sjögren’s syndrome, systemic sclerosis, inflammatory myopathies, systemic vasculitides, giant cell arteritis, Horton disease, cranial arteritis, temporal arteritis, T-cell based immunotherapy induced cytokine storm, chimeric antigen receptor T-cell therapy induced cytokine storm, immune effector cell-associated neurotoxicity syndrome, T-cell bispecific antibody therapy induced cytokine storm, pulmonary infiltrate, adult respiratory distress syndrome, interstitial lung disease, pneumonia, community acquired pneumonia, and acute interstitial pneumonia.

In some embodiments, a method of designing a γc-cytokine antagonist peptide and/or a derivative thereof configured to modulate and/or block signaling by at least one of IL-2, IL-9, and IL-15 γc-cytokine family member that inhibits, ameliorates, reduces a severity of, treats, delays the onset of, or prevents at least one cytokine storm related disorder comprises the steps of using a computer to obtain from an amino acid sequence database amino acid sequences of at least one IL-2 and IL-15 γc-cytokine family members, assembling a γc cytokine antagonist peptide and/or a derivative thereof based on a sequence of the at least one IL-2 and IL-15 γc-cytokine family members, wherein the γc cytokine antagonist peptide and/or the derivative thereof modulates and/or blocks signaling by the at least one of IL-2, IL-9, and IL-15 γc-cytokine family members.

In some embodiments of the method of designing a γc-cytokine antagonist peptide and/or a derivative thereof, the γc cytokine antagonist peptide comprises a partial sequence of a γc-box D-helix region of each of at least IL-2 and IL-15 γc-cytokine family members.

In some embodiments of the method of designing a γc-cytokine antagonist peptide and/or a derivative thereof, the sequence comprises consecutive blocks of at least 5 amino acids of the γc-box D-helix region of each of at least IL-2 and IL-15 γc-cytokine family members.

In some embodiments of the method of designing a γc-cytokine antagonist peptide and/or a derivative thereof, the sequence comprises consecutive blocks of 1-10 amino acids of the γc-box D-helix region of each of at least IL-2 and IL-15 γc-cytokine family members.

In some embodiments of the method of designing a γc-cytokine antagonist peptide and/or a derivative thereof, the γc cytokine antagonist peptide comprises 11 to 50 amino acids.

In some embodiments of the method of designing a γc-cytokine antagonist peptide and/or a derivative thereof, the γc cytokine antagonist peptide further comprises a conjugate at the N-termini, C-termini, side residues, or a combination thereof.

In some embodiments of the method of designing a γc-cytokine antagonist peptide and/or a derivative thereof, the γc cytokine antagonist peptide further comprises a signal peptide.

In some embodiments of the method of designing a γc-cytokine antagonist peptide and/or a derivative thereof, the γc cytokine antagonist peptide comprises a sequence of SEQ ID NO: 1 (BNZ-γ)

In some embodiments of the method of designing a γc-cytokine antagonist peptide and/or a derivative thereof, the γc cytokine antagonist peptide derivative shares at least about 60% identity with a peptide of SEQ ID NO: 1.

In some embodiments of the method of designing a γc-cytokine antagonist peptide and/or a derivative thereof, the γc cytokine antagonist peptide derivative shares at least about 90% identity with a peptide of SEQ ID NO: 1.

In some embodiments of the method of designing a γc-cytokine antagonist peptide and/or a derivative thereof, the γc cytokine antagonist peptide derivative shares at least about 95% identity with a peptide of SEQ ID NO: 1.

In some embodiments of the method of designing a γc-cytokine antagonist peptide and/or a derivative thereof, the γc cytokine antagonist peptide and the derivative thereof have similar physico-chemical properties but distinct IL-2, IL-9, and IL-15 biological activities.

In some embodiments, a kit for inhibiting, ameliorating, reducing a severity of, treating, delaying the onset of, or preventing at least one cytokine storm related disorder comprises one or more of the compositions provided herein.

In some embodiments of the kit, the at least one cytokine storm related disorder is selected from the group consisting of cytokine release syndrome, cytokine storm, multiple organ dysfunction syndrome, systemic inflammatory response syndrome, sepsis, septic shock, graft-versus-host disease, haploidentical donor transplantation, sarcoidosis, hemophagocytic lymphohistiocytosis, vascular leak syndrome, systemic capillary leak syndrome, Stevens-Johnson syndrome, toxic epidermal necrolysis, asthmatic allergic lung inflammation, rhinosinusitis, viral infection, coronavirus infection, multi-system inflammatory syndrome in children (MIS-C) associated with COVID-19 (or another coronavirus disease), viral hemorrhagic fever, influenza viral infection, hantaviral infection, Epstein-Barr viral infection, HIV/HCV coinfection liver fibrosis, fungal infection, pulmonary Aspergillosis, bacterial infection, toxic shock syndrome, lyme neuroborreliosis, lyme disease, autoimmune disease, juvenile idiopathic arthritis, Still’s disease, macrophage activation syndrome, Sjögren’s syndrome, systemic sclerosis, inflammatory myopathies, systemic vasculitides, giant cell arteritis, Horton disease, cranial arteritis, temporal arteritis, T-cell based immunotherapy induced cytokine storm, chimeric antigen receptor T-cell therapy induced cytokine storm, immune effector cell-associated neurotoxicity syndrome, T-cell bispecific antibody therapy induced cytokine storm, pulmonary infiltrate, adult respiratory distress syndrome, interstitial lung disease, pneumonia, community acquired pneumonia, and acute interstitial pneumonia.

### BRIEF DESCRIPTION OF THE DRAWINGS

FIG. 1A shows an alignment of the D-helix region of human γc-cytokine family members.

FIG. 1B depicts the γc-box (SEQ ID NO: 8) and IL-2/IL-15 box (SEQ ID NO: 9) motifs which give rise to the consensus sequence around the D-helix region of the γc-cytokines.

FIG. 2 depicts a diagramed representation of the biochemical properties of amino acids.

FIG. 3A shows inhibition of IL-15, and IL-9 activity by BNZ-γ in a PT-18 proliferation assay.

FIG. 3B shows a proliferation assay of CTLL-2 cells grown in the presence of IL-2 or IL-15 and 0, 0.1, 1 or 10 µM BNZ-γ.

FIG. 4 shows inhibition of IL-15-mediated tyrosine-phosphorylation of STAT5 by BNZ-γ.

FIG. 5 shows schematic of cytokine storm animal system to study BNZ-γ inhibitory effects on cytokine storm.

FIG. 6 shows that BNZ-γ at 2 mg/kg administered twice weekly was 100% protective of cytokine storm-induced mortality in mice. The protective effect of BNZ-γ is statistically significant (P=0.008).

FIG. 7 shows that BNZ-γ at 2 mg/kg administered twice per week drastically reduced pro-inflammatory cytokine plasma levels within 7 days of viral challenge and blocking onset of cytokine storm lethality in mice.

### DETAILED DESCRIPTION

Some embodiments herein relate to compositions, methods, and kits comprising one or more therapeutic compounds that modulate signaling by at least one of IL-2, IL-9, and IL-15 γc-cytokine family member for inhibiting, ameliorating, reducing a severity of, treating, delaying the onset of, or preventing immune diseases such as cytokine-release syndrome, and cytokine storm associated disorders. Cytokines of the γc-family comprise a group of mammalian cytokines that are mainly produced by epithelial, stromal and immune cells and control the normal and pathological activation of a diverse array of lymphocytes. Descriptions of target diseases, as well as methods of administration, production, and commercialization of the therapeutic compounds are disclosed.

### Overview

More than 100 cytokines have been identified so far and are considered to have developed by means of gene duplications from a pool of primordial genes (See Bazan, J.F. 1990, Immunol. Today 11:350-4). In support of this view, it is common for a group of cytokines to share a component in their multi-subunit receptor system. The most well-documented shared cytokine subunit in T cells is the common γ subunit (yc-subunit).

The γc-subunit is shared by 6 known cytokines (Interleukin-2 (IL-2), Interleukin-4 (IL-4), Interleukin-7 (IL-7), Interleukin-9 (IL-9), Interleukin-15 (IL-15), and Interleukin-21 (IL-21), collectively called the “yc-cytokines” or “γc-family cytokines” and plays an indispensable role in transducing cell activation signals for all these cytokines. Additionally, for each of the γc-cytokines, there are one or two private cytokine-specific receptor subunits that when complexed with the γc-subunit, give rise to a fully functional receptor (Rochman et al., 2009, Nat Rev Immunol 9: 480-90).

The γc-family cytokines are a group of mammalian cytokines that are mainly produced by epithelial, stromal and immune cells and control the normal and pathological activation of a diverse array of lymphocytes. These cytokines are critically required for the early development of T cells in the thymus as well as their homeostasis in the periphery. For example, in the absence of the γc-subunit, T, B and NK cells do not develop in mice (Sugamura et al., 1996, Annu Rev Immunol 14:179-205).

The γc-cytokines are important players in the development of the lymphoid cells that constitute the immune system, particularly T, B, and NK cells. Further, γc-cytokines have been implicated in various human diseases. Thus, factors that inhibit γc-cytokine activity would provide useful tools to elucidate the developmental mechanism of subsets of lymphocytes and to treat immune disorders and γc-cytokine-mediated diseases.

Germ line depletion of the genes encoding the γc-subunit in mice or mutations of γc-subunit in humans are known to cause severe combined immunodeficiency (SCID) by disrupting the normal appearance or function of NK, T, and B cells. The importance of the γc-subunit in the signal transduction of the γc-cytokines, IL-2, -4, -7, -9, 15, -21, is indicated in studies demonstrating the lack of response of lymphocytes from these mice and human patients to the γc-cytokines (Sugamura et al., 1995, Adv Immunol 59:225-77). This indicates that disruption of the interaction between the γc-subunit and a γc-cytokine would efficiently block the intracellular signaling events by the γc-cytokine family members. Therefore, antagonist peptides according to some embodiments disclosed herein are expected to effectively block the pathogenic changes in humans suffering from the diseases mediated by misregulation of the IL-2, IL-9, or IL-15 γc-cytokine family members.

Applicants present novel compositions, methods, and kits comprising one or more therapeutic compounds that modulate signaling by at least one IL-2, IL-9, and IL-15 γc-cytokine family member for inhibiting, ameliorating, reducing a severity of, treating, delaying the onset of, or preventing immune diseases such as cytokine-release syndrome, and cytokine storm associated disorders. Applicants have also devised novel, low molecular weight therapeutic compounds herein referred to as “Simul-Block”, which suppress the activity of IL-2, IL-9, and IL-15 γc-cytokines. These low molecular weight therapeutic compounds, which include both chemicals and peptides, are often less immunogenic than antibodies, and can be used as a stand-alone approach, or complementary to antibody-mediated or small-molecule-mediated approaches, for modulating IL-2, IL-9, and/or IL-15 γc-cytokine activity in clinical interventions.

### Pathologies Associated With the IL-2, IL-9, and IL-15 γc-Cytokines

Recent studies have indicated that dysregulation of expression and dysfunction of the IL-2, IL-9, or IL-15 γc-cytokines could lead to a wide variety of human immunologic and hematopoietic diseases.

### IL-2

While IL-2 was historically considered a prototype T cell growth factor, the generation of a knockout mouse lacking IL-2 expression revealed that IL-2 is not critical for the growth or developmental of conventional T cells in vivo. Over-expression of IL-2, however, leads to a preferential expansion of a subset of T-cells; the regulatory T cells (T-regs) (Antony et al., 2006, J Immunol 176:5255-66). T-regs suppress the immune responses of other cells and thus act to maintain peripheral tolerance (Sakaguchi et al., 2008, Cell 133:775-87). Breakdown of peripheral tolerance is thought to cause autoimmune diseases in humans.

Thus, the immunosuppressive function of T-regs is thought to prevent the development of autoimmune diseases (Sakaguchi et al., 2008, Cell 133:775-87). T-regs have also been implicated in cancer, where solid tumors and hematologic malignancies have been associated with elevated numbers of T-regs (De Rezende et al., 2010, Arch Immunol Ther Exp 58:179-90).

### IL-9

The role of IL-9 is still rather uncharacterized compared to other γc-cytokine family members. Mice depleted of the IL-9 gene appear normal and do not lack any subsets of cells in the lymphoid and hematopoietic compartments. Recent studies, however, reveal an in vivo role for IL-9 in the generation of Th17 (T-helper induced by interleukin-17) cells (Littman et al., 2010, Cell 140:845-58; Nowak et al., 2009, J Exp Med 206:1653-60).

### IL-15

IL-15 is critically involved in the development of NK cells, NK-T cells, some subsets of intraepithelial lymphocytes (IELs), γδ-T cells, and memory-phenotype CD8 T-cells (Waldmann, 2007, J Clin Immunol 27:1-18; Tagaya et al., 1996, EMBO J 15:4928-39). Over-expression of IL-15 in mice leads to the development of NK-T cell and CD8 cell type T cell leukemia (Fehniger et al., 2001, J Exp Med 193:219-31; Sato et al. 2011, Blood 117:4032-40). These experimentally induced leukemias appear similar to LGL (large-granular lymphocyte) leukemia in humans, since in both instances the leukemic cells express CD8 antigen.

It is also suspected that IL-15-mediated autocrine mechanisms may be involved in the leukemic transformation of CD4 T lymphocytes (Azimi et al., 1998, Proc Natl Acad Sci 95:2452-7; Azimi et al., 1999, J Immunol 163:4064-72; Azimi et al., 2000, AIDS Res Hum Retroviruses 16:1717-22; Azimi et al., 2001, Proc Natl Acad Sci 98:14559-64). For example, CD4-tropic HTLV-I, which causes Adult T cell leukemia in humans, induces autocrine growth of virus-transformed T cells through the production of IL-15 and IL-15Rα (Azimi et al., 1998, Proc Natl Acad Sci 95:2452-7).

In addition to leukemic transformation, recent studies implicate IL-15 in the pathological development of Celiac disease (CD), an autoimmune disease. IL-15 is known to stimulate the differentiation of NK, CD8 and intestinal intraepithelial lymphocyte (IEL) cells into lymphokine-activated killer (LAK) cells by inducing the expression of cytolytic enzymes (i.e., Granzyme and Perforin) as well as interferon-y. CD is an immune-mediated enteropathy that is triggered by the consumption of gluten-containing food in individuals that express specific HLA-DQ alleles.

The prevalence of this disease is 1% in the western population. The only current treatment for CD is the complete elimination of gluten from the patient’s diet. The pathology of CD is mainly caused by extensive damage to the intestinal mucosa, which is caused by activated CD8 T cells that have infiltrated to the intestinal lamina propria. These CD8 T cells appear to be activated through mechanisms involving IL-15. One recent publication demonstrated in mice that ectopic over-expression of IL-15 by enterocytes leads to the development of enteropathy, which closely resembles the lesions in CD patients. Neutralization of IL-15 activity dramatically diminished the pathological changes. Thus, an intervention blocking the activation of CD8 T cells by IL-15 appears to provide an alternative strategy in managing CD to the conventional gluten-free diet.

### Current Strategies for Treating γc-Cytokine-Mediated Disorders

Because the γc-cytokines are thought to be involved in numerous human diseases, several methods of treating γc-cytokine-implicated diseases by inhibiting γc-cytokine family activities have been proposed. These methods include the use of cytokine-specific monoclonal antibodies to neutralize the targeted cytokine’s activity in vivo; use of monoclonal antibodies targeting the private cytokine-specific receptor subunits (subunits other than the shared γc-subunit) to selectively inhibit cytokine activity; and use of chemical inhibitors that block the downstream intracellular cytokine signal transduction pathway.

While cytokine-specific antibodies are often the first choice in designing therapeutics, cytokines that share receptor components display overlapping functions (Paul, W.E., 1989, Cell 57:521-4) and more than one cytokine can co-operate to cause a disease (See Examples described herein). Thus, approaches involving neutralization of a single cytokine may not be effective in the treatment of cytokine-implicated human diseases.

Strategies for designing therapeutics that inhibit the function of multiple cytokines via antibodies which recognize a shared receptor component have also been proposed. However, the multi-subunit nature of cytokine receptor systems and the fact that functional receptors for a single cytokine can assume different configurations makes this approach difficult.

For example, a functional IL-15 receptor can be either IL-15Rβ/γc or IL-15Rα/β/γc (Dubois et al., 2002, Immunity 17:537-47). An antibody against the IL-15Rβ receptor (TMβ1), is an efficient inhibitor of the IL-15 function, but only when the IL-15Rα molecule is absent from the receptor complex (Tanaka et al., 1991, J Immunol 147:2222-8). Thus, the effectiveness of a monoclonal anti-receptor antibody, whether raised against a shared or a private subunit, can be context-dependent and is unpredictable in vivo.

Although clinical use of monoclonal antibodies against biologically active factors or receptors associated with the pathogenesis of diseases is an established practice, there are few demonstrations of successful outcomes. Moreover, establishment of a clinically-suited monoclonal antibody treatment is a long and difficult process, with the successful generation of a neutralizing antibody largely a matter of luck. For example, due to the critical importance of the γc-subunit in mediating signaling by γc-family cytokines, many attempts to generate polyclonal and monoclonal antibodies against the γc-subunit have been made and there exist many commercial antibodies recognizing the γc-subunit in mice and humans. Curiously, however, none of these anti- γc-subunit antibodies block the function of the γc-cytokines.

Another problem with the therapeutic use of monoclonal antibodies is that monoclonal antibodies are usually generated by immunizing rodents with human proteins, so the generated antibody is a foreign protein and thus highly immunogenic. To circumvent this problem, the amino acid sequence of the monoclonal antibody is molecularly modified so that the antibody molecule is recognized as a human immunoglobulin (a process called humanization), but this process requires time and expense.

### Targeting JAK3, As an Existing Alternative Example for the Inhibition of Multiple γc-Cytokines

The interaction between the γc-subunit and a γc-cytokine leads to the activation of an intracellular protein tyrosine kinase called Janus kinase 3 (Jak3). Jak3, in turn, phosphorylates multiple signaling molecules including STAT5, and PI3 kinase. The interaction of the γc-subunit and Jak3 is very specific. In fact, there is no other receptor molecule that recruits Jak3 for signal transduction (O′Shea, 2004, Ann Rheum Dis 63:(suppl. II):ii67-7). Thus, the inhibition of cytokine signaling through the γc-subunit can be accomplished by blocking the activity of Jak3 kinase. Accordingly, multiple small molecule chemical inhibitors that target the kinase activity of Jak3 have been introduced to the market (Pesu et al., 2008, Immunol Rev 223:132-42). One such example is CP690,550.

The major shortcoming of these protein kinase inhibitors is the lack of specificity to Jak3 kinase. These drugs intercept the binding of ATP (adenosine-triphosphate) molecules to Jak3 kinase, a common biochemical reaction for many protein kinases, and thus tend to block the action of multiple intracellular protein kinases that are unrelated to Jak3 kinase whose actions are critically needed for the well-being of normal cells in various tissues. Thus, more specific inhibitors of signaling through the γc-subunit are needed.

There is therefore a great need for an alternative non-small molecule chemical strategy for treating γc-cytokine-implicated diseases.

### Discovery of the Γc-Box

The C-terminus (the D-helix) of the γc-cytokines contains the proposed site for interacting with the common γc-subunit of the multi-unit cytokine receptors (Bernard et al., 2004 J Biol Chem 279:24313-21). Comparison of the biochemical properties of the amino acids of all γc-cytokines identified in mice and humans revealed that the chemical nature of the amino acids, for example, hydrophobicity, hydrophilicity, base/acidic nature, are conserved, if not identical, at many positions in the D-helix across the members of the γc-cytokine family.

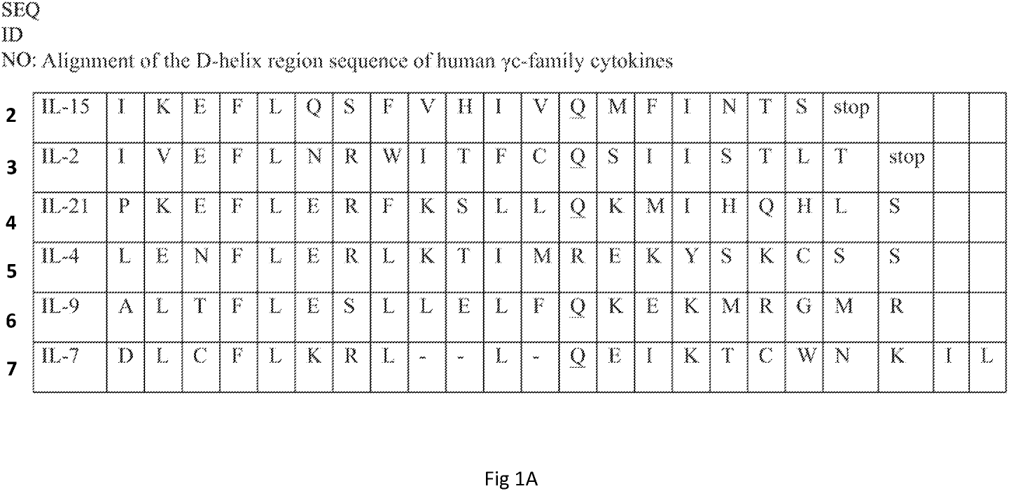

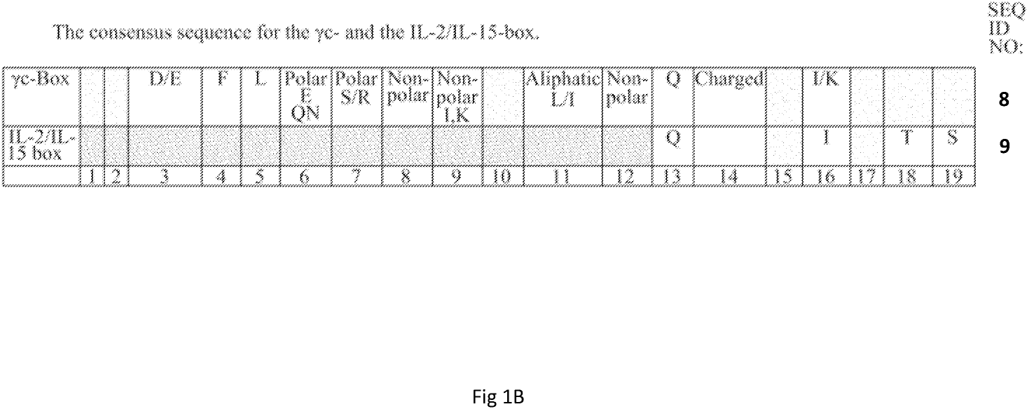

In contrast, the sequence of IL-13, which is related to the γc-cytokine, IL-4, but does not bind to the γc-subunit, does not exhibit significant homology in the D-helix region to the γc-cytokines, suggesting that the sequence homology in the D-helix region is correlated with binding to the γc-subunit. As shown in FIG. 1A, alignment of the amino acid sequences of the D-helix region of γc-cytokine family members in humans reveals a motif of moderate sequence homology in these cytokines referred to herein as “the γc-box”.

The γc-box (SEQ ID NO: 8) comprises 19 amino acids where out of the 19 positions, positions 4, 5, and 13 are fully conserved as Phenylalanine, Leucine, and Glutamine, respectively. Less conservation is observed at positions 6, 7 and 11 of the γc-box where the amino acid is one of two or three related amino acids that share physico-chemical properties: position 6 may be occupied by the polar amino acids Glutamate, Asparagine or Glutamine; non-polar amino acids Serine or Arginine can occupy position 7; and position 11 is occupied by either of the non-polar aliphatic amino acids Leucine or Isoleucine. Positions 9 and 16 may be occupied by the either the non-polar amino acid Isoleucine or the polar amino acid Lysine. See FIG. 1B. Some differences in the amino acid composition of the γc-box are observed at positions 9 and 16 amongst subfamilies of the γc-cytokines. Comparison of the γc-cytokines across species indicates that Isoleucine is often present at the 9 and 16 positions in the IL-2/15 subfamily, whereas the other γc-family members often possess Lysine in these positions. Not wishing to be bound by a particular theory, Isoleucine and Lysine are biochemically different and thus may impart specific conformational differences between the IL-2/15 subfamily and other γc-cytokines.

Conservation of the γc-box motif between γc-cytokines is supported by findings that a Glutamine (Gln, Q) residue located in the D-helix region is critical for the binding of the γc-cytokines to the γc-subunit (Bernard et al., 2004 J Biol Chem 279: 24313-21).

### Modulators of γc-Cytokine Activity

The activity of γc-family cytokines may be blocked by disrupting the interaction between the γc-cytokine and the γc-subunit, for example by introducing a competitive inhibitor which can interact with the γc-subunit without stimulating signaling through the multi-subunit cytokine receptors. Not to be bound by a particular theory, the conserved γc-box motif, which participates in binding of the γc-family cytokines to the γc-subunit, presents a core base amino acid sequence which can be utilized to design peptide modulators of γc-cytokine signaling.

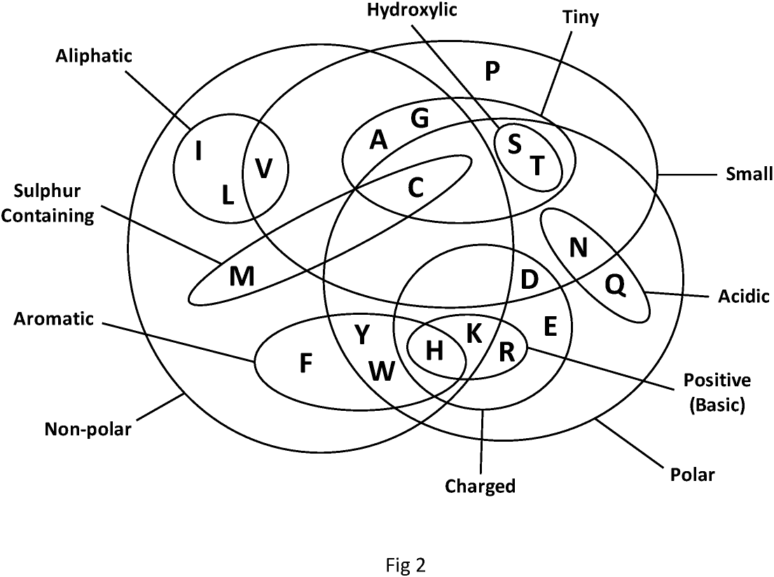

Based on the identification of the conserved γc-box motif in cytokines which bind to the γc-subunit, Applicants have devised a novel, 19-mer custom derivative peptide which is an artificial composite peptide combining the amino acid sequence of the human IL-2 and IL-15 γc-box. The 19-mer peptide, herein referred to as BNZ-γ, consists of the amino acid sequence: I-K-E-F-L-Q-R-F-I-H-I-V-Q-S-I-I-N-T-S (SEQ ID NO: 1), where the amino acids depicted by bold characters are conserved between IL-2 and IL-15 and the underlined amino acids represent positions where the physico-chemical properties of the amino acids are conserved (see FIG. 2).

In some embodiments, γc-cytokine antagonist peptides and derivatives thereof, which are also referred to herein as custom derivative peptides or composite peptide derivatives, of the 19-mer BNZ-γ amino acid sequence, I-K-E-F-L-Q-R-F-I-H-I-V-Q-S-I-I-N-T-S (SEQ ID NO: 1), can inhibit the activity of one or more IL-2, IL-9, and IL-15 γc-cytokines. Custom peptide derivatives of the 19-mer BNZ-γ amino acid sequence include any peptide whose partial amino acid sequence shows approximately 60-70%, 70-80%, 80%, 90%, 95%, 97%, 98%, 99% or 99.8% identity to amino acid sequence: I-K-E-F-L-Q-R-F-I-H-I-V-Q-S-I-I-N-T-S (SEQ ID NO: 1). Custom peptide derivatives of the 19-mer BNZ-γ amino acid sequence include any peptide whose partial amino acid sequence shows a % identity to I-K-E-F-L-Q-R-F-I-H-I-V-Q-S-I-I-N-T-S (SEQ ID NO: 1) that is equal to or at least about: 60%, 70%, 80%, 90%, 95%, 97%, 98%, 99%, 99.8%, or ranges including and/or spanning the aforementioned values. For example, custom peptides may share between 60% and 90% identity to SEQ ID NO:1, between 60% and 99% identity to SEQ ID NO:1, between 75% and 99% identity to SEQ ID NO:1, between 60% and 80% identity to SEQ ID NO:1, etc. Custom peptide derivatives further include any peptide wherein a partial amino acid sequence of that peptide derivative comprises amino acids with similar physico-chemical properties to the amino acids of sequence: I-K-E-F-L-Q-R-F-I-H-I-V-Q-S-I-I-N-T-S (SEQ ID NO: 1). For example, amino acids with similar physico-chemical properties would include Phenylalanine, Tyrosine, Tryptophan, and Histidine, which are aromatic amino acids. FIG. 2 shows a diagrammed representation of amino acids with similar physico-chemical properties which may be may be substituted for the amino acids of sequence: I-K-E-F-L-Q-R-F-I-H-I-V-Q-S-I-I-N-T-S (SEQ ID NO: 1).

In several embodiments, the amino acid residues of the custom derivative peptides retain similar physico-chemical properties with the amino acid residues of BNZ-γ, but exhibit different biological inhibition specificity to the IL-2, IL-9, and IL-15 γc-cytokine family members from that of the original 19-mer peptide. Peptide derivatives of BNZ-γ may be 19, 20, 21, 22, 23, 24, 25-30, 30-35, 35-40, 40-45, 45-50, or more than 50 amino acids in length. Peptide derivatives of BNZ-γ may have an amino acid length that is equal to or at least 19, 20, 21, 22, 23, 24, 25, 30, 35, 40, 45, 50, or ranges including and/or spanning the aforementioned values.

In some embodiments, the custom peptide derivatives may be conjugated to the N-termini, C-termini and/or to the side residues of existing biological proteins/peptides. In some embodiments, peptide derivatives of BNZ-γ may be conjugated to other moieties through the N-terminus, C-terminus, or side chains of the composite peptide. The other moieties may include proteins or peptides that stabilize the composite peptide, or other moieties, including without limitation, bovine serum albumin (BSA), albumin, Keyhole Limpet Hemocyanin (KLH), Fc region of IgG, a biological protein that functions as scaffold, an antibody against a cell-specific antigen, a receptor, a ligand, a metal ion and Poly Ethylene Glycol (PEG).

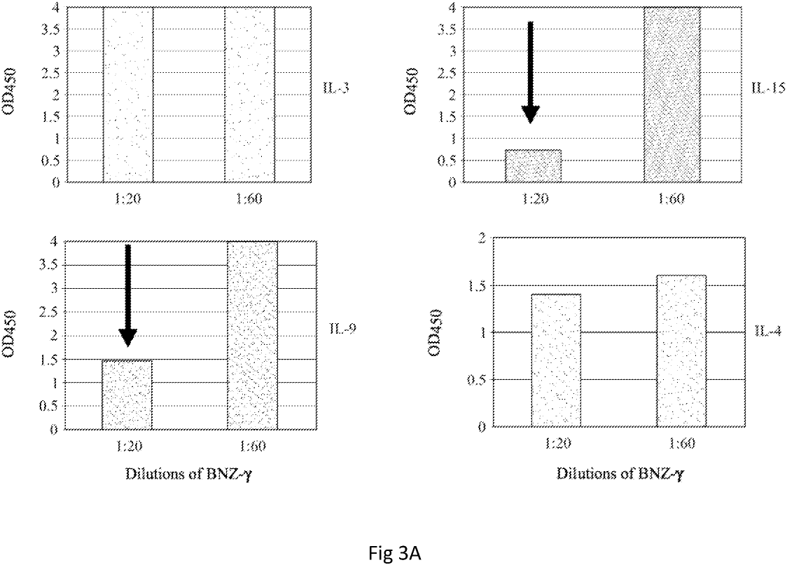

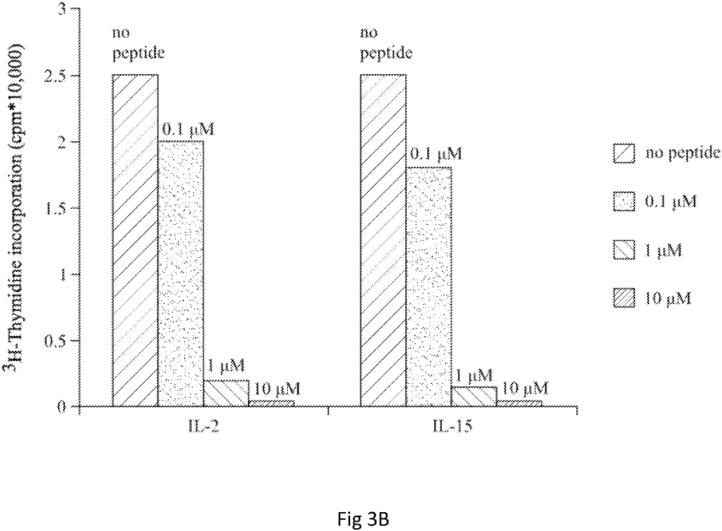

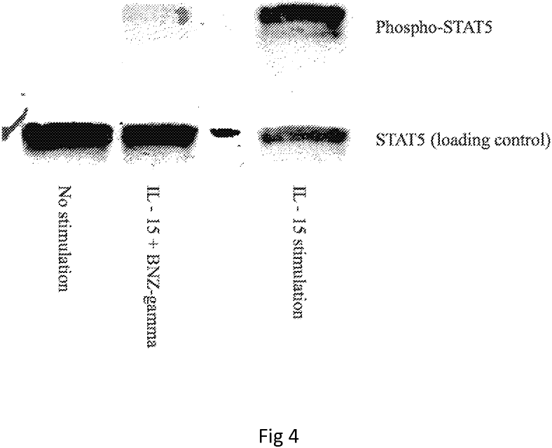

Applicants discovered that the 19-mer BNZ-γ, suppresses IL-2, IL-15 and IL-9 induced cellular proliferation, but not IL-3 or IL-4 induced cellular proliferation. See FIG. 3A, FIG. 3B, and EXAMPLE 2. Applicants further demonstrated that BNZ-γ inhibits IL-15 mediated phosphorylation of the intracellular cytokine signal transduction molecule, STAT-5. See FIG. 4 and EXAMPLE 5. These results demonstrate that custom peptide derivatives of the conserved IL-2 and IL-15 γc-box motif can modulate the activity of IL-2, IL-9, and IL-15 γc-cytokines.

Several embodiments relate to one or more therapeutic compounds that modulate signaling by at least one of IL-2, IL-9, and IL-15 γc-cytokine family member for inhibiting, ameliorating, reducing a severity of, treating, delaying the onset of, or preventing immune diseases such as cytokine-release syndrome, and cytokine storm associated disorders. In some embodiments, the therapeutic compound is one or more of a γc-cytokine antagonist peptide, a γc-cytokine antagonist peptide derivative, or a combination thereof. In some embodiments, the therapeutic compound is the 19-mer BNZ-γ (SEQ ID NO: 1), custom peptide derivatives of the 19-mer BNZ-γ (as disclosed elsewhere herein), and/or combinations thereof.

In some embodiments, any of the custom peptide derivatives disclosed herein can comprise one or more intra-peptide hydrocarbon linker elements. In some embodiments, the 19-mer BNZ-γ (SEQ ID NO: 1) comprises one or more intra-peptide hydrocarbon linker elements. In some embodiments, the 19-mer BNZ-γ (SEQ ID NO: 1) comprises one or more intra-peptide hydrocarbon linker elements that connect two separate amino acids positioned 4 residues apart on SEQ ID NO: 1. In some embodiments, the 19-mer BNZ-γ (SEQ ID NO: 1) comprises one or more intra-peptide hydrocarbon linker elements that connect two separate amino acids positioned 7 residues apart on SEQ ID NO: 1. In some embodiments, the 19-mer BNZ-γ (SEQ ID NO: 1) comprises one or more intra-peptide hydrocarbon linker elements that connect two separate amino acids positioned 4 residues apart on SEQ ID NO: 1 and 7 residues apart on SEQ ID NO: 1.

Several embodiments relate to custom peptide derivatives of the γc-box motifs of IL-15 or IL-2, which are depicted in FIG. 1A. Other embodiments relate to custom derivative peptides which are artificial composite peptides combining the amino acid sequence of human IL-15 and IL-2 γc-box motifs. Several embodiments relate to custom peptide derivatives of the γc-box motifs of IL-15 or IL-2 having a partial amino acid sequence that shows approximately 60-70%, 70-80%, 80%, 90%, 95%, 97%, 98%, 99% or 99.8% identity to amino acid sequences of the of the γc-box motifs of IL-15 or IL-2. In some embodiments, the custom peptide derivatives of the γc-box motifs of IL-15 or IL-2 include any peptide whose partial amino acid sequence shares a % identity with the amino acid sequences of the γc-box motifs of IL-15 or IL-2 that is equal to or at least about: 60%, 70%, 80%, 90%, 95%, 97%, 98%, 99%, 99.8%, or ranges including and/or spanning the aforementioned values. Custom peptide derivatives of the of the γc-box motifs of IL-15 or IL-2 further include any peptide wherein a partial amino acid sequence of that peptide derivative comprises amino acids with similar physico-chemical properties to the amino acids of sequence of the γc-box motifs of IL-15 or IL-2.

Several embodiments relate to custom peptide derivatives that would inhibit the function of one, all, or selective members of the IL-2, IL-9, and IL-15 γc-cytokines. In some embodiments, the custom peptide derivatives selectively target individual IL-2, IL-9, or IL-15 γc-cytokine family members. For example, a custom peptide derivative can selectively inhibit the function of IL-2, IL-9, or IL-15. In other embodiments, a custom peptide derivative can inhibit 2 or more IL-2, IL-9, and IL-15 γc-cytokine family members.

For example, the custom peptide derivatives of the present embodiments can selectively inhibit the function of IL-2 in combination with one or more of IL-9 and IL-15; IL-9 in combination with one or more of IL-2 and IL-15; or IL-15 in combination with one or more of IL-2 and IL-9. In other embodiments, custom peptide derivatives can comprehensively target all IL-2, IL-9, and IL-15 γc-cytokine family members.

Not wishing to be bound by a particular theory, the custom peptide derivatives can inhibit the function of all or selective members of the IL-2, IL-9, and IL-15 γc-cytokines by diminishing the binding of IL-2, IL-9, and/or IL-15 γc-cytokines to the γc-subunit, for example, as a competitive inhibitor. Such custom peptide derivatives may be used in diverse applications, including as a clinical drug.

Several embodiments relate to custom peptide derivatives that would modulate (including enhance or reduce) the function of one, two, or more of selective members of the IL-2, IL-9, and IL-15 γc-cytokines. In some embodiments, the custom peptide derivatives selectively target individual IL-2, IL-9, and IL-15 γc-cytokine family members. For example, a custom peptide derivative can selectively enhance or inhibit the function of IL-2, IL-9, or IL-15. In other embodiments, a custom peptide derivative can enhance or inhibit two or more IL-2, IL-9, and IL-15 γc-cytokine family members.

In some embodiments, one or more of the custom peptide derivatives of the conserved IL-2 and/or IL-15 γc-box motif disclosed herein can inhibit the activity of one or more IL-2, IL-9, and IL-15 γc-cytokines. In some embodiments, one or more of the custom peptide derivatives of the conserved IL-2 and/or IL-15 γc-box motif disclosed herein can inhibit the activity of one or more IL-2, IL-9, and IL-15 γc-cytokines by suppressing cell proliferation induced by the one or more IL-2, IL-9, and IL-15 γc-cytokines. In some embodiments, one or more of the custom peptide derivatives of the conserved IL-2 and/or IL-15 γc-box motif disclosed herein can inhibit the activity of one or more IL-2, IL-9, and IL-15 γc-cytokines by inhibiting phosphorylation of the intracellular cytokine signal transduction molecule mediated by the one or more IL-2, IL-9, and IL-15 γc-cytokines. In some embodiments, one or more of the custom peptide derivatives of the conserved IL-2 and/or IL-15 γc-box motif disclosed herein can inhibit the activity of one or more IL-2, IL-9, and IL-15 γc-cytokines by suppressing cell proliferation induced by the one or more IL-2, IL-9, and IL-15 γc-cytokines and by inhibiting phosphorylation of the intracellular cytokine signal transduction molecule mediated by the one or more IL-2, IL-9, and IL-15 γc-cytokines. In some embodiments, one or more of the custom peptide derivatives of the IL-2 and/or IL-15 conserved γc-box motif disclosed herein can inhibit the activity of one or more IL-2, IL-9, and IL-15 γc-cytokines by one or more other mechanisms.

In some embodiments, one or more of the peptide sequences disclosed herein suppress proliferation of one or more cell types induced by one or more of the cytokines disclosed herein (e.g., IL-2, IL-9, and IL-15). In some embodiments, one or more of the peptide sequences disclosed herein suppress proliferation of one or more cell types induced by the IL-2, IL-9, or IL-15 cytokines disclosed herein. In some embodiments, one or more of the peptide sequences disclosed herein suppress proliferation of one or more cell types induced by some but not all of the IL-2, IL-9, and IL-15 cytokines disclosed herein. In some embodiments, SEQ ID NO: 1 suppresses IL-2, IL-9, and IL-15 induced cellular proliferation.

In some embodiments, one or more of the custom peptide derivatives of the conserved IL-2 and/or IL-15 γc-box motif disclosed herein can inhibit the activity of one or more IL-2, IL-9, and IL-15 γc-cytokines by inhibiting phosphorylation of one or more intracellular cytokine signal transduction molecules mediated by the one or more IL-2, IL-9, and IL-15 γc-cytokines disclosed herein. In some embodiments, one or more of the custom peptide derivatives of the conserved IL-2 and/or IL-15 γc-box motif disclosed herein can inhibit phosphorylation of one or more intracellular cytokine signal transduction molecules mediated by all of the IL-2, IL-9, and IL-15 γc-cytokines disclosed herein. In some embodiments, one or more of the custom peptide derivatives of the conserved IL-2 and/or IL-15 γc-box motif disclosed herein can inhibit phosphorylation of one or more intracellular cytokine signal transduction molecules mediated by some but not all of the IL-2, IL-9, and IL-15 γc-cytokines disclosed herein.

Also, for example, the peptides as disclosed herein may be used to inhibit IL-15 mediated phosphorylation of the intracellular cytokine signal transduction molecule STAT-5.

Provided herein are composite peptides, and compositions, methods, and kits to modulate IL-2, IL-9, and/or IL-15 γc-cytokine signaling. The terms “composite peptide,” “composite peptide derivative,” “custom peptide,” “antagonist peptides,” “antagonist peptides derivatives,” “oligopeptide,” “polypeptide,” “peptide,” and “protein” can be used interchangeably when referring to the “custom peptide derivatives” provided in accordance with the present embodiments and can be used to designate a series of amino acid residues of any length. The peptides of the present embodiments may be linear or cyclic. The peptides of the present embodiments may include natural amino acids, non-natural amino acids, amino acids in the (D) stereochemical configuration, amino acids in the (L) stereochemical configuration, amino acids in the (R) stereochemical configuration, amino acids in the (S) stereochemical configuration, or a combination thereof.

Peptides of the present embodiments may also contain one or more rare amino acids (such as 4-hydroxyproline or hydroxylysine), organic acids or amides and/or derivatives of common amino acids, such as amino acids having the C-terminal carboxylate esterified (e.g., benzyl, methyl or ethyl ester) or amidated and/or having modifications of the N-terminal amino group (e.g., acetylation or alkoxycarbonylamino), with or without any of a wide variety of side chain modifications and/or substitutions. Side chain modifications, substitutions or a combination thereof that may be present in the custom peptide derivatives of the present embodiments include, but are not limited to, α-methyl, α-alkenyl, alkylation, methylation, benzylation, t-butylation, tosylation, alkoxycarbonylamino, and the like.

Residues other than common amino acids that may be present include, but are not limited to, penicillamine, tetramethylene cysteine, pentamethylene cysteine, mercaptopropionic acid, norleucine, pentamethylene-mercaptopropionic acid, 2-mercaptobenzene, 2-mercaptoaniline, 2-mercaptoproline, ornithine, aminoisobutyric acid, diaminobutyric acid, aminoadipic acid, m-aminomethylbenzoic acid, and diaminopropionic acid.

Peptides of the present embodiments can be produced and obtained by various methods known to those skilled in the art. For example, the peptide may be produced by genetic engineering, based on the nucleotide sequence coding for the peptide of the present embodiments, or chemically synthesized by means of peptide solid-phase synthesis and the like, or produced and obtained in their combination. One skilled in the art of solid-phase peptide synthesis can readily incorporate natural or non-natural amino acids in the (D) as well as (L), or the (R) as well as (S), stereochemical configuration. It will also be apparent to one skilled in the art of solid-phase peptide synthesis to produce and obtain peptides containing one or more intra-peptide hydrocarbon linker elements of the present embodiments utilizing α-substituted (such as α-alkenyl) natural or non-natural amino acids in one or more of (D), (L), (R) or (S), stereochemical configurations, or a combination thereof. In some embodiments, an intra-peptide hydrocarbon linker element linking α-substituted amino acids (e.g., α-alkenyl amino acids) can be generated by catalyzing one or more ring-closing metathesis. In some embodiments, one or more intra-peptide hydrocarbon linker elements can be generated by catalyzing a ring-closing metathesis using benzylidenebis(tricyclohexyl-phosphine)-dichlororuthenium (Grubb’s catalyst) on the resin-bound peptide during peptide synthesis. In some embodiments, other ring-closing synthesis reactions and/or mechanisms during one or more known peptide synthesis processes are also contemplated. One skilled in the art can synthesize the custom peptide derivatives based on the present disclosure of the conserved γc-box motif and knowledge of the biochemical properties of amino acids as described in FIG. 2.

Peptides of the present embodiments may also comprise two or more α-alkenyl substituted amino acids. In some embodiments, the two or more α-alkenyl substituted amino acids are linked via one or more intra-peptide hydrocarbon linker elements incorporated at the α-alkenyl substituted amino acids. In some embodiments, the α-alkenyl substituted amino acids are utilized to catalyze the formation of an intra-peptide hydrocarbon linker element by ring-closing metathesis during peptide synthesis. Intra-peptide linker elements join separate amino acids on the same sequence of a custom peptide derivative of the present disclosure. In some embodiments, the peptides of the present disclosure are linear or cyclic.

In some embodiments, one or more intra-peptide hydrocarbon linker elements are incorporated at amino acid positions that correlate with a single α-helical turn in a secondary structure of the composite peptide. In some embodiments, when the composite peptide comprises one or more non-contiguous single α-helical turns, the amino acid positions that correlate with a single α-helical turn of the composite peptide correspond to amino acid positions i and i+4 of the composite peptide, where i is the first amino acid position of the single α-helical turn and i+4 is the last amino acid position of the single α- helical turn, and wherein amino acid positions i and i+4 comprise alpha-alkenyl substituted amino acids, and where i and i+4 are positioned 4 residues apart (4 spaced).

In some embodiments, one skilled in the art of solid-phase peptide synthesis can readily synthesize composite peptides comprising more than one intra-peptide hydrocarbon linker elements such that the composite peptide comprises more than one single α-helical turn. In some embodiments, the more than one single α-helical turns are non- contiguous, i.e., the more than one single α-helical turns do not share a substituted amino acid. For example, in some embodiments, the composite peptide can comprise one or more intra-peptide hydrocarbon linker elements of Formula 1 (See TABLE 1) that span more than one non-contiguous single α-helical turns of the composite peptide.

Not wishing to be bound to any specific peptide containing one or more intra-peptide hydrocarbon linker elements of the present embodiments, a generic peptide example containing one intra-peptide hydrocarbon linker element connecting two separate amino acids positioned 4 residues apart, or one α-helical turn (position i and position i+4), can have S-pentenylalanine (S5Ala) incorporated at each of the positions i and i+4 during solid-phase synthesis of the peptide before catalyzing ring-closing metathesis using Grubb’s catalyst while the peptide is still resin-bound on the solid support. This will result in a peptide sequence containing the intra-peptide hydrocarbon linker element depicted below (SEQ ID NO: 10) positioned 4 residues apart:

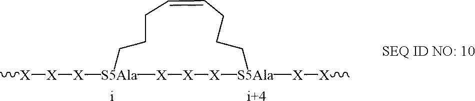

In some embodiments, one or more intra-peptide hydrocarbon linker elements are incorporated at amino acid positions that correlate with a double α-helical turn in a secondary structure of the composite peptide. In some embodiments, when the composite peptide comprises one or more non-contiguous double α-helical turns, the amino acid positions that correlate with a double α-helical turn of the composite peptide correspond to amino acid positions i and i+7 of the composite peptide, where i is the first amino acid position of the double α-helical turn and i+7 is the last amino acid position of the double α- helical turn, and wherein amino acid positions i and i+7 comprise alpha-alkenyl substituted amino acids, and where i and i+7 are positioned 7 residues apart (7 spaced).

Not wishing to be bound to any specific peptide containing one or more intra-peptide hydrocarbon linker elements of the present embodiments, a generic peptide example containing one intra-peptide hydrocarbon linker element connecting two separate amino acids positioned 7 residues apart, or two α-helical turns (position i and position i+7), can have R-octenylalanine (R8Ala) incorporated at position i and S-pentenylalanine (S5Ala) incorporated at position i+7 during solid-phase synthesis of the peptide before catalyzing ring-closing metathesis using Grubb’s catalyst while the peptide is still resin-bound on the solid support. This will result in a peptide sequence containing the intra-peptide hydrocarbon linker elements depicted below (SEQ ID NO: 11) positioned 7 residues apart:

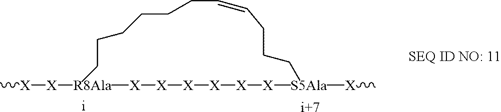

In some embodiments, one skilled in the art of solid-phase peptide synthesis can readily synthesize composite peptides comprising more than one intra-peptide hydrocarbon linker elements such that the composite peptide comprises more than one double α-helical turn. In some embodiments, the more than one double α-helical turns are non-contiguous, i.e., the more than one double α-helical turns do not share a substituted amino acid. For example, in some embodiments, the composite peptide can comprise one or more intra-peptide hydrocarbon linker elements of Formula 2 (See TABLE 1) that span more than one non-contiguous double α-helical turns of the composite peptide.

One skilled in the art of solid-phase peptide synthesis can readily synthesize peptides containing more than one intra-peptide hydrocarbon linker element of the present embodiments by incorporating α-alkenyl substituted amino acids at paired non-overlapping amino acid positions in the peptide, with each α-alkenyl substituted amino acid in the pair positioned a single α-helical turn apart (4 residues apart) or a double α-helical turn apart (7 residues apart) during solid-phase peptide synthesis before catalyzing ring-closing metathesis using Grubb’s catalyst while the peptide is still resin-bound on the solid support. In some embodiments, single peptides can comprise more than one intra-peptide hydrocarbon linker element that span a single α-helical turn (4 residues apart), can contain hydrocarbon linker elements that span a double α-helical turn (7 residues apart), or can contain a combination of both a single α-helical turn (4 residues apart) and a double α-helical turn (7 residues apart) intra-peptide hydrocarbon linker elements.

Peptides containing one or more intra-peptide hydrocarbon linker elements of the present embodiments can be produced through solid-phase peptide synthesis utilizing commercially available Boc- or Fmoc-protected α-alkenyl substituted natural or non-natural amino acids in the (D) as well as (L), or the (R) as well as (S), stereochemical configuration. The Fmoc-protected α-alkenyl substituted amino acids and the resultant hydrocarbon linker element following ring-closing metathesis that may be used in the synthesis of the custom peptide derivatives of the present embodiments include, but are not limited to Table 1:

TABLE 1

| α-alkenylSubstituted Amino | α-alkenyl Substituted |
|---|---|
| Acid | Amino Acid |
| Peptide Position i | Peptide Position i+4 |
| S-pentenylalanine (CAS: 288617-73-2; S5Ala) | S5Ala |
| Hydrocarbon Linker Element Following Ring-Closing Metathesis | |
| 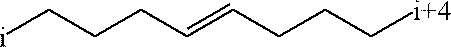 | |
| Formula 1 | |
| Peptide Position i | Peptide Position i+7 |
| R-octenylalanine (CAS: 945212-26-0; R8Ala) | S5Ala |

Hydrocarbon Linker Element Following Ring-Closing Metathesis

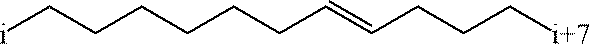

Formula 2

In some embodiments, an intra-peptide hydrocarbon linker can be further functionalized through one or more chemical reactions. In some embodiments, one or more carbon-carbon double bond(s) present in the intra-peptide hydrocarbon linker (e.g., Formula 1 - Formula 2 in TABLE 1) can be utilized for organic chemical reactions to add one or more additional chemical functionalities. For example, alkene reactions may be utilized for custom peptide derivatives that contain one or more intra-peptide hydrocarbon linker elements of the present embodiments. Non-limiting examples of alkene reactions include hydroboration, oxymercuration, hydration, chlorination, bromination, addition of HF, HBr, HCl or HI, dihydroxylation, epoxidation, hydrogenation, and cyclopropanation. In some embodiments, one or more additional chemical functionalities of the intra-peptide hydrocarbon linker elements can be achieved subsequent to the alkene reaction. Non-limiting examples include covalent addition of one or more chemical group substituents, such as nucleophilic reactions with epoxide and hydroxyl groups, and the like. In some embodiments, alkene reactions may be utilized to attach biotin, radioisotopes, therapeutic agents (non-limiting examples include rapamycin, vinblastine, taxol, etc.), non-protein fluorescent chemical groups (non-limiting examples include FITC, hydrazide, rhodamine, maleimide, etc.), and protein fluorescent groups (non-limiting examples include GFP, YFP, mCherry, etc.) to one or more inter- and/or intra-peptide hydrocarbon linker elements of the present embodiments.

Non-limiting examples of composite peptides comprising one or more intra-peptide hydrocarbon linker elements are provided in TABLE 2. The examples in TABLE 2 are not limiting with respect to any specific α-alkenyl substituted amino acid useful for the synthesis of single α-helical turn (4 spaced) and/or double α-helical turn (7 spaced) intra-peptide hydrocarbon linker elements of the present embodiments and/or to any specific amino acid stereochemical configuration (e.g., (D) stereochemical configuration denoted with “d” in TABLE 2) in the custom peptide derivatives of the present embodiments.

TABLE 2

SEQ ID NO

| | |
|---|---|
| {S5Ala }-I-K-E-{S5Ala }-L-Q-R-F-I-H-I-V-Q-S-I-I-N-T-S | 12 |
| I-K-E-F-L-Q-R-{S5Ala}-I-H-I-{S5Ala}-Q-S-1-1-N-T-S | 13 |
| I-K-E-F-L-Q-R-{R8Ala}-I-H-I-V-Q-S-{S5Ala}-I-N-T-S | 14 |
| I-K-E-F-L-Q-R-F-I-H-I-{S5Ala}-Q-S-I-{S5Ala}-N-T-S | 15 |
| I-K-E-F-L-Q-R-F-I-H-I-{R8Ala}-Q-S-I-I-N-T-{S5Ala} | 16 |
| {S5Ala₁}-I-K-E-{S5Ala₁}-L-Q-R-{S5Ala₂}-I-H-I-{S5Ala₂}-Q-S-I-I-N-T-S | 17 |
| {S5Ala₁}-I-K-E-{S5Ala₁}-L-Q-R-{R8Ala₂}-I-H-I-V-Q-S-{S5Ala₂}-I-N-T-S | 18 |
| {S5Ala₁}-I-K-E-{S5Ala₁}-L-Q-R-F-I-H-I-{S5Ala₂}-Q-S-1-{S5Ala₂}-N-T-S | 19 |
| {S5Ala₁}-I-K-E-{S5Ala₁}-L-Q-R-F-I-H-I-{R8Ala₂}-Q-S-I-I-N-T-{S5Ala₂} | 20 |
| {S5Ala₁}-I-K-E-{S5Ala₁}-L-Q-R-{S5Ala₂}-I-H-I-{S5Ala₂}-Q-S-I-I-{dN}-{dT}-{dS} | 21 |
| {S5Ala₁}-I-K-E-{S5Ala₁}-L-Q-R-{R8Ala₂}-I-H-I-V-Q-S-{S5Ala₂}-I-{dN}-{dT}-{dS} | 22 |
| {S5Ala₁}-I-K-E-{S5Ala₁}-L-Q-R-F-I-H-I-{S5Ala₂}-Q-S-I-{S5Ala₂}-{dN}-{dT}-{dS} | 23 |

*Subscript denotes corresponding pairs of hydrocarbon-linked α-alkenylsubstituted amino acids

Some embodiments also relate to polynucleotides comprising nucleotide sequences encoding the peptides of the present invention. “Nucleotide sequence,” “polynucleotide,” or “nucleic acid” can be used interchangeably, and are understood to mean either double-stranded DNA, a single-stranded DNA or products of transcription of the said DNAs (e.g., RNA molecules). Polynucleotides can be administered to cells or subjects and expressed by the cells or subjects, rather than administering the peptides themselves. Several embodiments also relate to genetic constructs comprising a polynucleotide sequence encoding the peptides of the present invention. Genetic constructs can also contain additional regulatory elements such as promoters and enhancers and, optionally, selectable markers.

### Methods of Treating Γc-Cytokine Mediated Diseases

Several embodiments relate to the use of therapeutic compounds, such as γc-antagonist peptides, and/or a derivatives thereof, to target the γc-subunit receptor whose activity and/or abundance may be directly modulated by IL-2, IL-9, and/or IL-15 cytokine signaling in the treatment of γc-cytokine mediated diseases. Use of the therapeutic compounds according to the present embodiments allows for flexibility in the design and combination, which enables more comprehensive outcomes that would not be accomplished by conventional strategies employing small-molecule chemical inhibitors or anti-cytokine receptor antibodies.

Described herein is a novel method of modulating the action of IL-2, IL-9, and/or IL-15 γc-family cytokines. Such manipulations can yield effective methods of clinical interventions in treating immune diseases such as cytokine-release syndrome, and cytokine storm associated disorders.

In some embodiments, compositions, methods, and kits for inhibiting, ameliorating, reducing a severity of, treating, delaying the onset of, or preventing at least one cytokine storm related disorder are described. In some embodiments, the therapeutic compounds described herein may be used for inhibiting, ameliorating, reducing a severity of, treating, delaying the onset of, or preventing one or more of cytokine release syndrome, cytokine storm, multiple organ dysfunction syndrome, systemic inflammatory response syndrome, sepsis, septic shock, graft-versus-host disease, haploidentical donor transplantation, sarcoidosis, hemophagocytic lymphohistiocytosis, vascular leak syndrome, systemic capillary leak syndrome, Stevens-Johnson syndrome, toxic epidermal necrolysis, asthmatic allergic lung inflammation, rhinosinusitis, viral infection, coronavirus infection, multi-system inflammatory syndrome in children (MIS-C) associated with COVID-19 (or other coronavirus diseases), viral hemorrhagic fever, influenza viral infection, hantaviral infection, Epstein-Barr viral infection, HIV/HCV coinfection liver fibrosis, fungal infection, pulmonary Aspergillosis, bacterial infection, toxic shock syndrome, lyme neuroborreliosis, lyme disease, autoimmune disease, juvenile idiopathic arthritis, Still’s disease, macrophage activation syndrome, Sjögren’s syndrome, systemic sclerosis, inflammatory myopathies, systemic vasculitides, giant cell arteritis, Horton disease, cranial arteritis, temporal arteritis, T-cell based immunotherapy induced cytokine storm, chimeric antigen receptor T-cell therapy induced cytokine storm, immune effector cell-associated neurotoxicity syndrome, T-cell bispecific antibody therapy induced cytokine storm, pulmonary infiltrate, adult respiratory distress syndrome, interstitial lung disease, pneumonia, community acquired pneumonia, and acute interstitial pneumonia.

Several embodiments relate to therapeutic compounds that would modulate the signaling of all or selective members of the IL-2, IL-9, and IL-15 γc-cytokines. In some embodiments, therapeutic compounds selectively modulate the signaling of individual IL-2, IL-9, or IL-15 γc-cytokine family members. In other embodiments, therapeutic compounds can comprehensively modulate the signaling of all IL-2, IL-9, and IL-15 γc-cytokine family members (Simul-Block). In some embodiments, therapeutic compounds can selectively modulate the signaling of subsets of the IL-2, IL-9, and IL-15 γc-cytokines. Not wishing to be bound by a particular theory, the therapeutic compounds can modulate the function of all or selective members of the IL-2, IL-9, and IL-15 γc-cytokines by diminishing the binding of the IL-2, IL-9, and/or IL-15 γc-cytokines to the γc-subunit, for example, as a competitive inhibitor.

### Cytokine Release Syndrome and Cytokine Storm Associated Disorders

As disclosed elsewhere herein, in some embodiments, the therapeutic compounds described herein may be used for inhibiting, ameliorating, reducing a severity of, treating, delaying the onset of, or preventing cytokine release syndrome and/or one or more cytokine storm associated disorders.

Multiple IL-2, IL-9, and/or IL-15 γc-cytokine family members have been implicated as being involved in cytokine release syndrome and cytokine storm associated disorders. Cytokine release syndrome describes an acute systemic inflammatory syndrome. Throughout the inflammatory response, the body tightly manages and regulates the response through a signaling balance between pro- and anti-inflammatory cytokine molecules. In cytokine release syndrome (also known as cytokine storm) and in cytokine storm associated disorders, the body experiences an imbalanced increase of pro-inflammatory cytokine signaling, further creating positive feedback to immune cells for increased production of pro-inflammatory cytokines, and can quickly result in a damaging systemic inflammatory immune response, severe illness, fever, multiple organ dysfunction syndrome, and eventual death (D′Elia, R.V. et al., 2013, Clin Vaccine Immunol 20:319-27; Tisoncik, J.R. et al., 2012, Microbiol Mol Biol Rev 76:16-32). Cytokine release syndrome and cytokine storm associated disorder etiologies are characterized by exogenous inflammatory insults including viral, bacterial, and fungal infections, as well as, non-infections conditions such as autoimmune diseases, pulmonary infiltrate conditions, T-cell based immunotherapies, antibody therapy, trauma, graft-versus-host disease, and numerous other examples described herein. IL-2, IL-9, and/or IL-15 are involved in the pathogenesis of various cytokine storm immuno-pathologies by driving the proliferation and survival of cytotoxic immune cells, and inducing the production of pro-inflammatory cytokines such as IL-6, IFN-y, TNFα, MCP-1, and GM-CSF (Agostini, C. et al., 1996, J Immunol 157:910-8; Chien, J. et al., 2006, Respirology 11:715-22; McKinstry, K.K. et al., 2019, PLoS Pathog 15:e1007989; Nakamura, R. et al., 2010, J Virol 84:5574-82). The IL-2, IL-9, and/or IL-15 γc-cytokines induce prolonged pro-inflammatory exaggeration and drive cytokine release syndrome and cytokine storm associated disorder pathogenesis.

Multiple organ dysfunction syndrome (MODS) refers to progressive organ dysfunction in an acutely ill patient, such that homeostasis cannot be maintained and requiring intervention. It is a severe outcome following infectious (sepsis, septic shock) and noninfectious conditions such as systemic inflammatory response syndrome (SIRS) associated with severe acute pancreatitis, surgery, or trauma. Elevated serum IL-15 correlates with MODS and is predictive of disease severity and mortality. IL-15 is elevated and has been observed in patients suffering from severe acute pancreatitis, patients suffering from postoperative sepsis, and severely injured trauma patients (Ueda, T. et al., 2007, Surgery 142:319-26; Kimura, A. et al., 2012, J Surg Res 175:e83-8; Cahill, L.A. et al., 2020, Injury 51:819-29). IL-15 also supports pathogenic natural killer cell proliferation and survival, thereby directly contributing to immuno-pathogenesis (Guo, Y. et al., 2015, J Immunol 195:2353-64; Guo, Y. et al., 2017, J Immunol 198:1320-33; Lamparello, A.J. et al., 2019, J Am Coll Surg 229:5310).

Sepsis is a clinical syndrome that is caused by a dysregulated host response to infection. Sepsis and the subsequent systemic inflammatory response can lead to multiple organ dysfunction syndrome and death. Septic shock is a type of distributive shock caused by vasodilation and impaired distribution of blood flow. Septic shock is defined as sepsis that has circulatory, cellular, and metabolic abnormalities that are associated with a greater risk of mortality than sepsis alone. Clinically, this includes patients who fulfill the criteria for sepsis who, despite adequate fluid resuscitation, require vasopressors to maintain a mean arterial pressure (Singer, M. et al., 2016, JAMA 315:801-10). Both IL-2 and IL-15 are elevated during septic shock. Septic shock affiliated with IL-2 was observed in patients with endotoxemia and has a concurrent up-regulation of TNFα and endotoxin (Endo, S. et al., 1992, Circ Shock 38:264-74; Blackwell, T.S. et al., 1996, Br J Anaesth 77:110-7). IL-15 has been implicated in disease pathogenesis by propagating NK cell function and production of IFN-γ, significantly exacerbating the severity of septic shock (Guo, Y. et al., 2015, J Immunol 195:2353-64; Guo, Y. et al., 2017, J Immunol 198:1320-33).

Graft-versus-host disease (GVHD) is the result of a complex immune response following allogeneic stimuli. GVHD occurs in an acute and/or chronic manner as a result of donor T cells (graft cells) recognizing the presence of histocompatibility antigens in the host that differ from those of the donor cells. This initial antigen recognition results in donor-derived T-cells differentiation into CD4 and CD8 effector cells with the production of pro-inflammatory cytokines and direct CD8 T-cell cytotoxic effects which are responsible for the inflammatory effects and host tissue damage associated with GVHD. As it is well known that members of the γc-cytokine family are involved in the activation of CD4 and CD8 T-cells, the positive association of a number of γc-cytokines with GVHD pathogenesis has been reported. Increased IL-2 production by donor CD4 T-lymphocytes is observed early in the induction of chronic and acute GVHD in preclinical animal models (Via, C.S. et al., 1991, J Immunol 146:2603-9; Antin, J.H. et al., 1992, Blood 80:2964-8). Animal studies determined that IL-2 is critical to the development of acute GVHD and results in the development of donor-anti-host cytotoxic T-lymphocytes and is unregulated in patients experiencing acute and chronic GVHD (Via, C.S. et al., 1993, Int Immunol 5:565-72; Hechinger, A.K. et al., 2015, Blood 125:570-80). The prophylactic use of two IL-2 receptor antagonistic antibodies showed beneficial effects on GVHD in hematologic malignancy patients following donor-peripheral blood stem cell transplantation (Fang et al., 2012, Biol Blood Marrow Transplant. 18:754-62). IL-15 is also an early marker of acute GVDH in patients, as serum levels of IL-15 have also been shown to elevate sharply in GVHD patients within the first month of post-transplantation (Chik et al. 2003, J Pediatr Hematol Oncol. 25:960-4; Thiant, S. et al., 2010, Bone Marrow Transplant 45:1546-52). In preclinical animal models, donor derived IL-15 has been shown to contribute to acute GVDH and is critical for disease progression in a CD8 T-cell dependent manner, while elimination of IL-15 prevents GVHD disease onset (Blaser et al., 2005, Blood 105:894-901; Blaser, B.W. et al., 2006, Blood 108:2463-9).

Allogeneic hematopoietic cell transplantation (HCT) is an effective therapy for a wide variety of hematopoietic malignancies and non-malignant hematologic disorders. The pluripotent hematopoietic stem cells required for are derived from the bone marrow or peripheral blood of a related or unrelated donor. The best results of allogeneic HCT have been obtained from (HLA)-matched siblings. However, HLA-matched donor availability can be limited, and the majority of patients generally have readily available half-matched haploidentical donors through their parents, referred to as haploidentical donor transplantation, or HLA-haploidentical HCT. The primary challenge of HLA-haploidentical HCT is severe bidirectional alloreactivity, which often leads to high incidences of graft rejection, GVHD, and severe cytokine release syndrome. IL-2 and IL-15 are each up-regulated and contribute to both GVHD and cytokine release syndrome. IL-2 and IL-15 have been shown to be up-regulated in patients suffering from severe cytokine release syndrome following HLA-haploidentical HCT and correlates with very poor survival (Abboud, R. et al., 2016, Biol Blood Marrow Transplant 22:1851-60; Yarkoni, S. et al., 2012, Biol Blood Marrow Transplant 18:523-35).

Sarcoidosis is a multi-organ disorder characterized by the accumulation of T lymphocytes, mononuclear phagocytes, and noncaseating granulomas in involved tissues. Approximately 90% of patients develop lung pathology and pulmonary disease, which accounts for the majority of the morbidity and mortality associated with this disease. Other tissues commonly involved include the skin, eyes, and lymph nodes. Studies from sarcoidosis patients determined that IL-2 is essential for the activation of lymphocyte populations within the lung by stimulating in situ proliferation or by cellular recruitment from the peripheral blood (Forrester, J.M. et al., 1994, J Immunol 153:4291-302; Agostini, C. et al., 1996, J Immunol 157:910-8; Vissinga, C. et al., 1996, Hum Immunol 48:98-106; Agostini, C. et al., 2000, Curr Opin Rheumatol 12:71-6; Prasse, A. et al., 2000, Clin Exp Immunol 122:241-8; Logan, T.F. et al., 2005, Thorax 60:610-1). Activated T-cells drive the recruitment and differentiation of alveolar macrophages leading to the spontaneous release of IFN-γ and TNFα by monocyte, NK cell, and lymphocyte populations (Robinson, B.W. et al., 1985, J Clin Invest 75:1488-95; Prior, C. et al., 1991, Am Rev Respir Dis 143:53-60; Prasse, A. et al., 2000, Clin Exp Immunol 122:241-8; Hao, W. et al., 2014, Proc Natl Acad Sci 111:16065-70). Additionally, IL-2 contributes to the development of hypergammaglobulinemia by promoting B-cell differentiation and an overproduction of immunoglobulin in patients (Hunninghake, G.W. et al., 1981, J Clin Invest 67:86-92; Agostini, C. et al., 2000, Curr Opin Rheumatol 12:71-6). During pulmonary sarcoidosis, IL-15 is produced by macrophages and synergizes with IL-2 and TNFα to stimulate pro-inflammatory and cytotoxic bronchoalveolar lavage fluid (BALF) T-cells contributing to T-cell alveolitis (Agostini, C. et al., 1996, J Immunol 157:910-8; Muro, S. et al., 2001, J Allergy Clin Immunol 108:970-5).

Hemophagocytic lymphohistiocytosis (HLH) is a fatal syndrome characterized by excessive immune activation. HLH occurs with higher frequency in infants from birth to 18 months of age, but has also been observed in children and adults of all ages. HLH can occur as a familial or sporadic disorder, and may be triggered by a variety of immune insults that disrupt immune homeostasis. Immune cell activation can be triggered by infection. Notably, an HLH-like syndrome has been associated with COVID-19 patients following SARS-CoV-2 infection (Mehta, P. et al., 2020, Lancet 395:1033-4). Other immune triggers include malignancy, or autoimmunity (Ramos-Casals, M. et al., 2014, Lancet 383:1503-16). HLH is propagated by primary or acquired defects in T-cell and natural killer cell cytotoxicity that preclude their ability to terminate the immune response (Brisse, E. et al., 2015, Cytokine Growth Factor Rev 26:263-80). Therefore, hypercytokinemia is both a result and driver of immune cell activation and involves elevations in IFN-y, TNFα, IL-2, and IL-6 (Zinter, M.S. et al., 2019, Blood 134:103-4). IL-2 has been implicated in the survival of pathogenic, apoptosis-resistant IL-2-activated lymphocytes and IL-2-driven cytokine storm during HLH (Fadeel, B. et al., 1999, Br J Haematol 106:406-15; Trottestam, H. et al., 2011, Blood 118:4577-84; Shaw, T.Y. et al., 2016, J Investig Med High Impact Case Rep 4:2324709616647409).

Vascular leak syndrome (VLS) and systemic capillary leak syndrome (SCLS or Clarkson’s disease) are overlapping nomenclature for a disease disorder characterized by severe episodes of hypotension, hypoalbuminemia, and hemoconcentration. The disorder is characterized by excessive vascular permeability, pulmonary edema, reduced blood oxygen concentrations, and acute kidney injury. VLS is most commonly associated with sepsis, but is also caused by severe trauma, reperfusion injury, venomous snake bites, acute lung injury, acute respiratory distress syndrome (ARDS), and burns (Duan, C. et al., 2017, Mil Med Res 4:11). Drug toxicity associated with IL-2 cancer therapy has also been shown to manifest in VLS pathology (Funke, I. et al., 1994, Ann Hematol 68:49-52; Lentsch, A.B. et al., 1999, Cancer Immunol Immunother 47:243-8; Lourdes, L.S. et al., 2012, Case Rep Hematol 2012:954201; Poust, J.C. et al., 2013, Anticancer Drugs 24:1-13; Xie, Z. et al., 2014, J Clin Cell Immunol 5:1000213). IL-2 induced VLS also results in cardiovascular symptoms similar to those of septic shock, with an increased heart rate and cardiac output, a decrease in systemic peripheral resistance, and documented presentation of coronary artery disease, ischemia, myocardial infarction, arrhythmias, ventricular and supraventricular arrhythmias, and death (White, R.L. et al., 1994, Cancer 74:3212-22; Tan, M.C. et al., 2016, J Cardiol Cases 15:28-31). IL-2 activates endothelial cells and mediates immune effector cells recruitment and neutrophilia, leading to the expression of IFN-y, TNFα, as well as nitric oxide and tissue damage (Wei, S. et al., 1993, J Immunol 150:1979-87; Orucevic, A. et al., 1998, Cancer Metastasis Rev 17:127-42; Rafi, A.Q. et al., 1998, J Immunol 161:3077-86; Jillella, A.P. et al., 2000, Leuk Lymphoma 38:419-22; Assier, E. et al., 2004, J Immunol 172:7661-8; Carey, P.D. et al., 1997, Surgery 122:918-26). IFN-γ in particular has been shown to be unregulated in the blood of patients within 6 hours following administration of IL-2 (Orucevic, A. et al., 1998, Cancer Metastasis Rev 17:127-42; Lentsch, A.B. et al., 1999, Cancer Immunol Immunother 47:243-8). Furthermore, increased Tac antigen expression, which identifies the β-chain of IL-2, was demonstrated on the perivascular blood mononuclear cells of symptomatic patients with SCLS. The increase of Tac-positive cells is significantly associated with vascular leak episodes and poor disease prognosis (Cicardi, M. et al., 1990, Ann Intern Med 113:475-7). Additionally, functional expression of the high-affinity IL-2 receptor complex on pulmonary endothelial cells is largely responsible for the IL-2 cytokine-mediated pathology. Importantly, IL-2-induces VLS in lymphocyte-depleted mice, indicating that this response is independent of NK, T, or B cells. IL-2Rα expression in lung endothelial cells is also increased by IL-2, suggesting a positive feedback loop, whereas inhibition of IL2Rα prevents symptoms of VLS. IL-2 signaling in these cells results in high nitric oxide secretion, a potent vasodilator that is elevated in VLS and is toxic to endothelial cells (Krieg, C. et al., 2010, Proc Natl Acad Sci 107:11906-11).

Stevens-Johnson syndrome (SJS) and toxic epidermal necrolysis (TEN) are severe mucocutaneous reactions caused by a dysregulated cellular immune response, often in response to medications. SJS and TEN are characterized by extensive necrosis and detachment of the epidermis. Both IL-2 and IL-15 are implicated in the cutaneous immuno-inflammation in SJS/TEN. Biopsies determined that IL-2 in the perivascular dermis of SJS/TEN patients likely supports the proliferation of pathogenic lymphocytes (Caproni, M. et al., 2006, Br J Dermatol 155:722-8; Chung, W.H. et al., 2012, J Dermatol Sci 66:190-6; Abe, R., 2015, J Dermatol 42:42-8). Elevated IL-15 in patient serum is also strongly associated with disease severity (Stern, R.S. et al., 2017, J Invest Dermatol 137:1004-8; Su, S.C. et al., 2017, J Invest Dermatol 137:1065-73), and is likely critical for the induction of pathogenic pro-inflammatory responses and the up-regulation of the pro-inflammatory cytokines TNFα, IL-6, IL-1, IL-8, GM-CSF, MIP-1α, and MIP-1β that are elevated in SJS/TEN patient serum (McInnes, I.B. et al., 1997, Nat Med 3:189-95; Kim, Y.S. et al., 1998, J Immunol 160:5742-8; Su, S.C. et al., 2017, J Invest Dermatol 137:1065-73). IL-15 also directly contributes to the immunopathology of SJS/TEN by being essential to the maintenance of long lasting cytotoxic T-cells, persistence of natural killer cells, and enhancing antigen presentation of MHC, which contributes to prolonged hypersensitivity (Zhang, X. et al., 1998, Immunity 8:591-9; Waldmann T.A. et al., 1999, Annu Rev Immunol 17:19-49; Becker, T.C. et al., 2002, J Exp Med 195:1541-8; Tourkova, I.L. et al., 2005, J Immunol 175:3045-52; Jabri, B. et al., 2015, Nat Rev Immunol 15:771-83).

Allergen insult to the weakened lung of an asthmatic patient can result in localized cytokine storm pathogenic conditions. Asthma is caused by chronic airway inflammation characterized by reversible airway obstruction, airway hyperresponsiveness (AHR), infiltration of eosinophils and type-2 T-cells into the airway submucosa, mucus hypersecretion, and airway remodeling. Allergic respiratory disease in adults is associated with active Th2 T-cell immune responses to inhaled allergens, in contrast with a Th1 immune phenotype in normal, healthy subjects. Lymphocytes from asthmatic patients display a higher abundance of the IL-2 private receptor chain (CD25) and are able to significantly increase eosinophil proliferation (Yang, J. et al., 1993, J Allergy Clin Immunol 91:792-801). Furthermore, animal models determined that IL-2 signaling contributes to AHR by directing resident, IL-2-dependent pathogenic Th2 memory cells that actively drive lung allergic responses (Hondowicz, B.D. et al., 2016, Immunity 44:155-66). Additionally, innate immune cell derived IL-2 is implicated in driving eosinophilic crystalline pneumonia in preclinical models (Roediger, B. et al., 2015, J Allergy Clin Immunol 136:1653-63). IL-9 is also a major pathogenic cytokine in the pathogenesis of allergic asthma. Preclinical models determined that IL-9 actively contributes to pulmonary eosinophilia, airway remodeling, mucus hypersecretion, and AHR after allergen challenge (Shimbara, A. et al., 2000, J Allergy Clin Immunol 105:108-15; Dong, Q. et al., 1999, Eur J Immunol 29:2130-9; Soussi-Gounni, A. et al., 2001, J Allergy Clin Immunol 107:575-82; Zhou, Y. et al., 2001, Respir Res 2:80-4). IL-9 induces the release of pathogenic IL-2 and IL-13 by mast cells and airway epithelial cells and promotes a shift towards a pathogenic Th2-immune phenotype (Temann, U.A. et al., 2002, J Clin Invest 109:29-39; Temann, U.A. et al., 2007, Int Immunol 19:1-10; Arras, M. et al., 2001, Am J Respir Cell Mol Biol 24:368-75; Barnes, P.J. et al., 2008, J Clin Invest 118:3546-56). PBMC derived T-cells from allergic asthma patients have also been shown to actively produce IL-9 (Jia, L. et al., 2017, BMC Immunol 18:38; Moretti, S. et al., 2017, Nat Commun 8:14017).

Rhinosinusitis is an inflammatory disorder of the mucous membranes of the nasal cavity and contiguous paranasal sinus mucosa as a result of infection, allergies, air pollution, and structural misalignment of the nose. If left unresolved the chronic inflammation can result in localized cytokine storm pathogenic conditions. Rhinosinusitis is categorized as occurring with or without nasal polyps. Serum cytokine studies have shown that IL-2 is up-regulated in rhinosinusitis patients without nasal polyps (Rai, G. et al., 2018, Ann Lab Med 38:125-31; Schlosser, R.J. et al., 2016, JAMA Otolaryngol Head Neck Surg 142:731-7). In rhinosinusitis patients with nasal polyps, biopsies and sinus mucosal specimens show an up-regulation of IL-9 expression as determined by immunohistochemistry and mRNA quantification (Bequignon, E. et al., 2020, J Transl Med 18:136; Lin, H. et al., 2015, Am J Rhinol Allergy 29:e18-23; Olcott, C.M. et al., 2016, Int Forum Allergy Rhinol 6:841-7).

### Infection Cytokine Storm Associated Disorders

As disclosed elsewhere herein, in some embodiments, the therapeutic compounds described herein may be used for inhibiting, ameliorating, reducing a severity of, treating, delaying the onset of, or preventing cytokine storm associated disorders caused by infection.

Coronaviruses are detrimental human pathogens that are responsible for ~33% of all community-acquired respiratory tract infections in adults and children and can induce cytokine storm pathogenic conditions. Human coronaviruses can be divided into low pathogenic and high pathogenic coronaviruses. Notable high pathogenic coronaviral diseases are the Middle East respiratory syndrome, induced by MERS-CoV infection, severe acute respiratory syndrome, induced by SARS-CoV infection, and COVID-19, induced by SARS-CoV-2 infection. These viruses infect the lower respiratory tract and cause massive inflammatory cell infiltration and elevated pro-inflammatory cytokine/chemokine responses resulting in acute lung injury (ALI), acute respiratory distress syndrome (ARDS), and fatal pneumonia.

Serum IL-15 is a predictive biomarker for disease severity for coronavirus induced viral bronchiolitis (Leahy et al., 2015, Eur Resp J 47:212-22). Furthermore, IL-15 is markedly up-regulated in patients suffering from MERS infection, and is strongly associated with neurovirulence of the virus (Li et al., 2004, J Virol 78:3398-406; Mahallawi et al., 2018, Cytokine 104:8-13). Elevated inflammatory cytokines and chemokines levels in patients are strongly correlated with poor disease prognosis, immunopathology and infiltration of pathogenic inflammatory cells into the lungs (Channappanavar et al., 2017, Semin Immunopathol 39:529-39). During SARS, early induction of IL-2 and subsequent overproduction of IL-6 are primary drivers of immuno-pathological processes involved in lung injury (Chien et al., 2006, Respirology 11:715-22). Furthermore, preclinical models of evaluating the immune responses of SARS-CoV noted a significant increase of IL-2 in the lungs of animals, correlating with an influx of effector T-lymphocyte and acute pneumonitis (Chen et al., 2009, J Virol 84:1289-1301). SARS-CoV infection of non-human primates determined a marked increase of IL-15 in lungs of younger animals (Clay et al., 2014, Immun Ageing 11:4; 1742-4933-11-4). Elevated plasma levels of IL-2, IL-9, and IL-15 have been recorded in patients suffering from COVID-19 following SARS-CoV-2 infection (Guo et al., 2020, Military Med Res 7:1; Huang et al., 2020, Lancet 395:497-506; Liu et al., 2020, J Med Virol 92:491-4; Liu et al., 2020, EBioMedicine 55:102763). Additionally, elevated IL-2 is found in severe COVID-19 cases requiring ICU intervention and is concurrent with increased neutrophil infiltration of the lung (Huang et al., 2020, Lancet 395:497-506; Liu et al., 2020, EBioMedicine 55:102763). IL-2 drives severe lung infiltration of pathogenic neutrophils during acute interstitial pneumonia and has been shown to prevent neutrophil apoptosis during coronaviral induced acute respiratory distress syndrome, propagating alveolar damage and pulmonary edema, poor blood oxygenation (hypoxia), and acute kidney injury indicative of vascular leak syndrome (Lesur et al., 2000, Crit Care Med 12:3814-22; Okamoto et al., 2002, Blood 99:1289-98; Yuki, K. et al., 2020, Clin Immunol 215:108427; Channappanavar et al., 2017, Semin Immunopathol 39:529-39; Guo, J. et al., 2020, J Am Heart Assoc 9:e0162219).

An emerging condition termed multisystem inflammatory syndrome in children (MIS-C) associated with COVID-19 has recently been identified in children following SARS-CoV-2 infection and/or exposure. Children with MIS-C have multiple organs that can show extreme inflammation, including the heart, lungs, kidneys, brain, skin, eyes, and/or gastrointestinal organs (cdc.gov/mis-c/), and can present symptoms including, but not limited to fever, abdominal pain, vomiting, diarrhea, shock, rash, trouble breathing, and bluish lips or face (Chiotos, K. et al., 2020, J Pediatric Infect Dis Soc in press). Information that is emerging details a hyperinflammatory response similar to what is observed in Kawasaki disease and, in some patients, can result in heart failure (Belhadjer, Z. et al., 2020, Circulation in press; Panupattanpong, S. et al., 2020, Cleve Clin J Med in press). The γc-cytokines IL-2 and IL-15 are well documented as pathogenic drivers in hyperinflammatory immune responses following SARS-CoV-2 infection and are likely upregulated in MIS-C patients. Furthermore, a study has shown that blocking TNFα, a proinflammatory cytokine that is induced by γc-cytokine signaling, displayed a therapeutic benefit in a MIS-C patient (Dolinger, M.T. et al., 2020, J Pediatr Gastroenterol Nutr in press), providing support that IL-2 and/or IL-15 inhibition will have therapeutic benefit for the treatment of MIS-C.

Viral Hemorrhagic Fevers (VHFs) are caused by four distinct viral families; Arenaviridae, Bunyaviridae, Filoviridae, and Flaviviridae, and are well documented to induce systemic inflammatory conditions. Notable members of each family include the arenavirus Lassa virus, the bunyaviruses Rift Valley fever virus and Crimean-Congo hemorrhagic fever virus, the flaviviruses Yellow Fever virus and Dengue Fever virus, and filoviviruses Ebolaviruses and Marburg virus. IL-2, IL-9, and IL-15 are linked to the pathogenic mechanisms of VHFs and are markers for disease severity and poor prognosis.

Lassa virus (LASV) is the causative agent of Lassa Hemorrhagic Fever. Elevated levels of IL-15 in preclinical LASV models are linked to loss of antigen presenting cells (APCs) via natural killer cell-mediated killing of dendritic cells and macrophages (Russier et al., 2014, J Virol 88:13811-20; Schaeffer et al., 2018, PLoS Pathog 14:e1007430; Schaeffer et al., 2019, Viruses 11:287). Dysregulation of APCs limits effector T-cell activation and increases viremia and disease severity (Baize et al., 2009, J Virol 83:5890-903). Preclinical models of Rift Valley fever virus showed the up-regulation of IL-2, IL-9, and IL-15 in fatal disease outcome (Ermler et al., 2013, J Virol 87:4846-60).

Elevated IL-2, IL-9, and IL-15 are clearly associated with fatal disease outcomes in preclinical models and adults infected with Crimean-Congo hemorrhagic fever virus (CCHF) (Papa et al., 2009, Clin Microbiology and Infection 16:843-7; Ozsurekci et al., 2013, J Med Virol 85:1955-9; Ruiz et al., 2013, Animal Models for the Study of Human Disease, 927-70; Papa et al., 2015, J Med Virol 88:21-7; Smith et al., 2019, PLoS Pathogens 15:e1008050; Welch at al., 2019, PLoS Pathog 15:e1008183). All three cytokines are linked to severity of the virus-induced cytokine storm during infection. Elevated IL-15 is observed in patients and preclinical models that suffered terminal Yellow fever virus infection. Research suggests that IL-15 is produced not by PBMCs, but rather by tissue from damaged organs. Via this mechanism, IL-15, alongside other pro-inflammatory cytokines, likely exacerbates tissue injury in the kidney and lymphoid organs in the absence of viral replication (Bae et al., 2008, J Infect Dis 197:1577-84; Engelmann et al., 2014, PLoS Negl Trop Dis 8:e0003295).

Elevated IL-2 and IL-15 in sera of patients suffering from Dengue Fever (DF) or progressive Dengue Hemorrhagic Fever (DHF) is implicated in disease severity. IL-2 elevation early during DF aids in the transition towards a pathogenic immune response observed in progressive DHF (Chaturvedi et al, 2000, FEMS Immunol Med Microbiol 28:183-8). Notably, the levels of IL-2 are higher in patients suffering from DHF compared to healthy controls (Kurane et al., 1991, J Clin Invest 88:1473-80). IL-15 is also a clear marker of DHF severity and poor disease prognosis (Firberg et al, 2018, PLoS Negl Trop Dis 12:e0006975; Patro et al., 2019, Viruses 88:34;v110100034).

Elevated IL-2 and IL-15 levels are associated with fatal Ebola virus (EBOV) infections of different EBOV strains (Villinger et al., 1999, J Infect Dis 179:S188-91; Sullivan et al., 2003, J Virol 77:9733-7; Wauguier et al., 2010, PLoS Negl Trop Dis 4:e0000837; Mcelroy et al., 2014, J Infect Dis 210:558-66; Falasca et al., 2015, Cell Death Differ 22:1250-9; Mcelroy et al., 2014, Proc Natl Acad Sci 112:4719-24; Banadyga et al, 2019, Open Forum Infect Dis 6:ofz046). EBOV binding and subsequent activation of Tim-1 signaling substitutes TCR-dependent activation signaling, leading to the secretion of IL-2 and other pro-inflammatory cytokines and cytokine release syndrome (Younan et al., 2017, MBio 8:00847-17; Younan et al., 2019, PLoS Pathog 15:e1008068). Similar to EBOV infection, IL-2 and IL-15 are elevated during Marburg infection in non-human primate models, indicating a pathogenic role of these γc-cytokine family members during infection (Bixler et al., 2015, Viruses 7:5489-507; Lin et al., 2015, J Virol 89:9875-85)

Influenza viruses are responsible for an acute respiratory illness of both the upper and/or lower respiratory tract and often lead to cytokine storm pathogenic conditions. Elevated IL-2, IL-9, and IL-15 levels in serum of patients suffering from Influenza A virus infection (IAV) and preclinical animal models are well documented. Notably, all three cytokines are markers of disease severity. During highly pathogenic H7N9 associated cytokine storm, IL-2 is significantly up-regulated in patients, and contributes to disease pathogenesis by recruitment of inflammatory cells to infected tissue (Chi et al., 2013, J Infect Dis 208:1962-7; Guo et al., 2015, Sci Rep 5:srep10942). Furthermore, memory T-cell responses producing IL-2 mediate potent lung inflammation and acute respiratory distress syndrome via a mechanism involving pro-inflammatory natural killer cells (McKinstry et al., 2019, PLoS Pathog 15:e1007989). NK cells are potent producers of IFN-γ and contribute to macrophage activation. Elevated IL-9 has similarly been implicated in pathogenic H7N9 infection and contributes to pathogenic inflammatory cell infiltration and mucous cell metaplasia (Buchwietz et al., 2007, Toxicol Pathol 35:424-35; Guo et al., 2015, Sci Rep 5:srep10942). IL-15 is significantly up-regulated in patients infected with H1N1 (Huang et al., 2013, Arch Virol 158:2267-72). IL-15 has been implicated in mediating lung pathogenesis during IVA by promoting the survival of antigen-specific cytotoxic CD8⁺ T-cells and inducing CD8⁺ T-cell production of cytotoxic molecules granzyme B and perforin and IFN-γ during infection. Collectively, IL-2, IL-9, and IL-15 each contribute in the pathogenesis of influenza infection (Nakamura et al., 2010, J Virol 84:5574-82).

Hantaviruses are a family of viruses that are primarily spread by rodents, with notable strains being the Sin Nombre, Puumala, and Andes hantaviruses. Hantaviruses are the etiological agents for hantavirus pulmonary syndrome (HPS, also referred to as hantavirus cardiopulmonary syndrome and hemorrhagic fever with renal syndrome). Infection is associated with cytokine storm immuno-pathogenesis, acute shock, and vascular leakage. IL-2 is up-regulated in serum of patients infected with hantavirus, and is associated with an early aberrant induction of pro-inflammatory immune cell subsets and disease severity (Sadeghi et al., 2011, BMC Immunol 12:65; Outinen et al., 2016, Infect Dis (Lond) 48:682-7; Maleki et al., 2019, J Infect Dis 219:1832-40). Tissue biopsies obtained at autopsy from patients with HPS determined IL-2 is elevated in lungs and spleens, supporting its immunopathogenic function (Mori et al., 1999, J Infect Dis 179:295-302). Notably, IL-2 treatment and elevated IL-2 is associated with capillary-leak syndrome. Puumala hantavirus infection also leads to the induction of persistent cytotoxic NK cells. Research has shown that Puumala infected NK cells up-regulate the expression and release IL-15 and IL-15Rα, induce other NK cells to be cytotoxic, and are resistant to NK cell lysis (Björkström et al., 2010, J Exp Med 208:13-21; Braun et al., 2014, PLoS Pathog 10:e1004521; Klingström et al., 2019, J Intern Med 285:510-23). Loss of IL-15 reduced the virally activated NK cells, indicating that IL-15 activated and prolongs the survival of pathogenic NK cells (Braun et al., 2014, PLoS Pathog 10:e1004521). Furthermore, elevated serum IL-15 is also associated with fatal disease outcome, making IL-15 a clear pathogenic host-factor contributing to disease outcome (Maleki et al., 2019, J Infect Dis 219:1832-40).

The Epstein-Barr Virus (EBV) is a ubiquitously disseminated gammaherpesivirus that is predominately B-cell tropic. Failure to control EBV infection leads to accumulation of activated immune cells and can development into life-threatening cytokine storm conditions (Cron, R.Q. et al. 2019 Cytokine Storm Syndrome. Cham: Springer International Publishing). Effector T-cells, natural killer cells, and invariant natural killer T (iNKT) cells contribute to the production of IL-2 and IL-15, which leads to the enhanced activation of NK cells and cytotoxic CD8 T-cells (Cron, R.Q. et al. 2019 Cytokine Storm Syndrome. Cham: Springer International Publishing). Furthermore, IL-2 levels are elevated in patients suffering from symptomatic EBV infection and EBV-associated hemophagocytic lymphohistiocytosis (Han, X.C. et al., 2017, J Crit Care 39:72-7; Hornef, M.W. et al., 1995, Clin Diagn Lab Immunol 2:209-13; Linde, A. et al., 1992, J Infect Dis 165:994-1000; Lotz, M. et al., 1986, J Immunol 136:3636-42), which results in a failure to clear infected B-cells and fuels T-cell driven immune activation, uncontrolled pro-inflammatory cytokine production, and a cytokine storm pathogenic environment. Furthermore, enhanced expression of IL-9 is observed in biopsies from EBV-associated cancer patients, which likely plays a role in the propagation of EBV-infected T-cells in patients (Yang, L. et al., 2004, Cancer Res 64:5332-7).

Approximately 33% of patients infected with human immunodeficiency virus (HIV) are co-infected with hepatitis C virus (HCV). Patients infected with HCV and HIV show an enhanced progressive liver fibrosis when compared to patients suffering only from chronic HCV and a predisposition for a cytokine storm hepatic environment (Kushner et al., 2013, PLoS One 8:e60387). Activated hepatic stellate cells (HSCs) mediate HCV-induced liver fibrosis. Enhanced activation of HSCs during HCV/HIV co-infection increases accumulated extracellular matrix deposits by HSCs, which causes liver fibrosis, increases cirrhosis, and leads to liver failure. In patients, HSC activation correlates with increased IL-15 serum abundance and expression by lymphocytes, indicating a clear pathogenic role for IL-15 (Allison et al., 2009, J Infect Dis 200:619-23; Veenhuis et al., 2017, Clin Infect Dis 64:589-96). Subsequent work analyzing liver biopsies from patients co-infected with HIV/HCV determined the presence of IL-15 rs10833 AA genotype, which was associated with advanced liver fibrosis, increased serum inflammatory-biomarkers, and sustained virological responses (Jiménez-Sousa et al., 2016, Liver Int 36:1258-66).

Pulmonary Aspergillos describes a set of pulmonary illnesses caused by allergy, airway or lung invasion, cutaneous infection, or extra-pulmonary dissemination by a species of Aspergillus fungus. Common Aspergillos fungal strains causing pulmonary complications include A. fumigatus, A. flavus, and A. terreus. Aspergillus species are found ubiquitously in nature, and inhalation of infectious conidia is a frequent occurrence. Subsequent tissue invasion is uncommon, but can occur in immunocompromised patients associated with hematologic malignancies, hematopoietic cell transplantation, or solid organ transplantation and result in pulmonary cytokine storm pathogenic conditions. Profiling functional cytokine gene polymorphisms of patients infected with Aspergillus implicated a high production of IL-15 as a biomarker of disease severity and susceptibility to chronic cavitary pulmonary aspergillosis (Sambatakou, H. et al., 2006, Int J Immunogenet 33:297-302; Smith, N.L.D. et al., 2014, Clin Microbiol Infect 20:O480-8). IL-15 modulates the function of polymorphonuclear leukocytes and stimulates the secretion of IL-8 in response to hyphae of Aspergillus species (Winn, R.M. et al., 2003, J Infect Dis 188:585-90). IL-15 also contributes the to the pathogenic production of IFN-γ by promoting natural killer cell activity (Strengell, M. et al., 2003, J Immunol 170:5464-9; Smith, N.L.D. et al., 2014, Immunology 143:499-511).

Toxic shock syndrome is a life-threatening disease complication following the result of immune recognition of bacterial toxins produced from bacteria in the Staphylococcus genus (staph) or Streptoccus genus (strep) often referred to as staph or strep infections, respectively. Toxins produced by both bacteria can result in cytokine storm immune responses, and patients often present severe fevers, hypertension, and can experience a sharp decline in health leading to multi-system organ failure. Staphlococcus aureus exotoxins are super-antigens capable of activating large numbers of T cells, resulting in massive cytokine production. Conventional T-cell activation follows antigen recognition and presentation by antigen-presenting cells (APCs). APCs process antigens and express them on the cell surface in complex with class II major histocompatibility complex (MHC), which in turn is recognized by an antigen-specific T cell receptor. In contrast, exotoxin super-antigens produced by S. aureus do not require processing by antigen-presenting cells, and can directly interact with the invariant region of the class II MHC molecule (Li, H. et al., 1998, Immunol Rev 163:177-86). T-cell activation by super-antigens leads to a massive, uncoordinated release of pro-inflammatory cytokines. The cytokine release is biphasic, with an initial release of IL-2 with IL-1, TNFα, and IL-6, followed by a gradual increase of IFN-γ and IL-12 (Faulkner, L. et al., 2005, J Immunol 175:6870-7; Silversides, J.A. et al., 2010, Curr Infect Dis Rep 12:392-400). In response to exotoxin super-antigens, human peripheral blood mononuclear cells secrete IL-2 and other pro-inflammatory cytokines including TNFα, IL-6, IFN-γ, as well as chemokine including MCP-1 (Kappler, J. et al., 1989, Science 244:811-3; Parsonnet, J. et al., 1989, Rev Infect Dis 11:S263-9; Krakauer, T. et al., 1999, Immunol Res 20:163-73; Faulkner, L. et al., 2005, J Immunol 175:6870-7; Silversides, J.A. et al., 2010, Curr Infect Dis Rep 12:392-400; Kimber, I. et al., 2013, Tox Sci 134:49-63). The essential role of IL-2 to toxic shock cytokine storm pathogenesis is evidenced by studies in preclinical animal models that show negation of toxic shock syndrome-associated symptoms following the loss of IL-2 (Uchiyama, T. et al., 1986, Microbiol Immunol 30:469-83; Tokman, M.G. et al., 1995, Shock 3:145-51; Kalyan, S. et al., 2004, J Infect Dis 189:1892-6; Khan, A.A. et al., 2009, PLoS One 4:e8473; Kimber, I. et al., 2013, Tox Sci 134:49-63).

Lyme neuroborreliosis is caused by as systemic infection caused by pathogenic spirochetes of the genus Borrelia. Approximately 10-15% of infected patients experience inoculation of the central nervous system following lyme infection which can lead to cytokine storm pathogenic conditions. Clinical manifestation of lyme neuroborreliosis includes meningitis, radiculitis, and peripheral facial palsy. IL-2 is up-regulated in the serum and cerebrospinal fluid of patients suffering from the disease likely contributing to meningeal inflammation, immune T-cell activation, and the production of proinflammatory cytokines such as IFN-γ (Cerar, T. et al., 2013, Clin Vaccine Immunol 20:1578-84; Pietikainen, A. et al., 2016, J Neuroinflammation 13:273; Rauer, S. et al., 2018, Dtsch Arztebl Int 115:751-6; Nordberg, M. et al., 2011, J Neuroimmunol 232:186-93).

### Autoimmune Disease Cytokine Storm Associated Disorders

As disclosed elsewhere herein, in some embodiments, the therapeutic compounds described herein may be used for inhibiting, ameliorating, reducing a severity of, treating, delaying the onset of, or preventing autoimmune disease cytokine storm associated disorders.

Juvenile idiopathic arthritis (JIA), also known as Still’s disease, is a chronic idiopathic inflammatory disorder associated with cytokine storm conditions that primarily involves the patient’s joints. JIA is the most common type of arthritis in children under the age of 16 years old. Increased IL-2 release from PBMCs likely contributes to disease pathogenesis by supporting the proliferation and survival of unusual T-cell phenotypes that infiltrate the joint fluid of patients (De Maria et al., 1987, Eur J Immunol 17:1815-9; Lashine et al., 2015, Lupus 24:240-7). IL-2 has also been implicated in the induction of macrophage activation syndrome (MAS). MAS is characterized by episodes of overwhelming inflammation that occurs most commonly in children suffering from JIA. High IL-2 levels are an early disease marker for MAS and strongly correlate with clinical status of JIA patients as well as anemia, hypertriglyceridemia, and hyperferritinemia (Schulert et al., 2014, Best Pract Res Clin Rheumatol 28:277-92; Lerkvaleekul et al., 2018, Open Access Rheumatol 10:117-28). Additionally, IL-2 likely modulates the function of cytotoxic neutrophils in patients (Jarvis et al., 2007, Pediatr Rheumatol Online 5:13). Increased IL-15 levels have also been found in the synovial fluid of JIA patients (Ruprecht et al., 2005, J Exp Med 201:1793-1803). IL-15 can abrogate the function of regulatory T-cells (Tregs), as well as prevent apoptosis of infiltrating pathogenic effector T-cells during synovitis (Smolewska et al., 2004, Scand J Rheumatol 33:7-12; Macaubas, 2009, Nat Rev Rheumatol 5:616-26).

Sjögren’s syndrome (SS) is a chronic, multisystem inflammatory disorder that is characterized by lymphocytic infiltration of salivary and lacrimal glands and results in diminished lacrimal and salivary gland function. Patients experience a combination of dry eyes (keratoconjunctivitis sicca) and dry mouth (xerostomia). Other disease manifestations may also occur, including dryness of the skin and other mucosal surfaces. Systemic extraglandular features include arthritis, nephritis, cytopenia, pneumonitis, hypergammaglobulinemia, specific autoantibodies, and vasculitis. Neurologic manifestations include peripheral neuropathy, myelopathy, and cognitive disturbances. There is a significant risk of lymphoma associated with SS. The pathogenesis of SS involves a complex interplay between innate and adaptive immune responses, leading to autoimmunity and chronic inflammation, which are essential to disease establishment and progression. IL-2 producing T-lymphocytes contribute to disease pathology (Youinou et al., 2011, Arthritis Res Ther 13:227). Analysis of infiltrating CD4+ T-lymphocytes, obtained from salivary gland biopsies, determined the up-regulation of IL-2 and IFN-γ by these cells, as well as enrichment of IL-2 in saliva and tears of SS patients (Fox et al., 1994, J Immunol 152:5532-9; Boumba et al., 1995, Br J Rheumatol 34:326-33; Streckfus et al., 2001, Clin Oral Investig 5:133-5; Chen et al., 2019, Sci Rep 9:7319). Additional evidence implicates IL-2 and IFN-γ producing B-lymphocytes in disease progression (Harris et al., 2000, Nat Immunol 1:475-82). Minor salivary gland biopsy also revealed that IL-15 is highly produced by salivary gland epithelial cells of SS patients (Sisto et al., 2016, Pathology 48:602-7). Furthermore, IL-15 mediated stimulation of T- and B-lymphocytes has been implicated in disease pathogenesis (Sisto et al., 2017, Clin Exp Med 17:341-50). IL-9 has also been recently identified to be up-regulated by salivary gland epithelial cells with SS following autoantibody treatment. This observation places IL-9 downstream of an NF-κB induced pro-inflammatory cytokine cascade, contributing to disease exacerbation (Lisi et al., 2012, Lab Invest 92:615-24).

Systemic sclerosis is a chronic multisystem inflammatory disease characterized by widespread vascular dysfunction and progressive fibrosis of the skin and internal organs (Pattanaik, D. et al., 2015, Front Immunol 6:272). IL-2, IL-9, and IL-15 are all associated with progressive immune activation and disease pathogenesis. The presence of IL-2 in sera of scleroderma patients strongly supports T-cell activation and is associated with disease progression and severity (Baraut, J. et al., 2010, Autoimmun Rev 10:65-73; Gourh, P. et al., 2009, Arthritis Rheum 60:3794-806; Kahaleh, M.B. et al., 1989, Ann Intern Med 110:446-50; Needleman, B.W. et al., 1992, Arthritis Rheum 35:67-72). IL-9 is elevated in skin and organ biopsies of systemic sclerosis patients and correlates with enhanced immune activation, immune cell tissue infiltration and toxicity, as well as disease severity (Guggino, G. et al., 2017, Clin Exp Immunol 190:208-16). IL-15 has been implicated as a marker for early disease onset and lung disease severity. Elevated levels of IL-15 in serum of patients correlated with impaired lung function, fibrotic and vascular lung disease, and vasculopathy in early disease pathogenesis (Wuttge, D.M. et al., 2007, Arthritis Res Ther 9:R85).

Inflammatory myopathies collectively describe a group of heterogenous autoimmune inflammatory disorders that manifest in the skeletal muscle and can progress to cytokine storm pathogenic conditions. Inflammatory myopathies include dermatomyositis, polymyositis, sporadic inclusion body myositis, and necrotizing autoimmune myopathy. Serum IL-2 is significantly up-regulated in patients suffering from dermatomyositis and polymyositis and has been implicated in propagating pro-inflammatory innate immune activity (De Paepe, B. et al., 2015, Int J Mol Sci 16:18683-713; Gono, T. et al., 2014, Rheumatology 53:2196-203). Both dermatomyositis and polymyositis are often complicated by rapidly progressive or chronic interstitial lung disease, which is improved with a reduction of serum IL-2 in these patients (Gono, T. et al., 2014, Rheumatology 53:2196-203). IL-15 has also been shown to directly contribute to disease pathogenesis. Over-expression of IL-15 in serum of patients is mediated by resident muscle cells which interact with infiltrating T-cells, and infiltrating macrophages (Baird, G.S. et al., 2008, Arch Pathol Lab Med 132:232-8; Notarnicola, A. et al., 2015, Scand J Rheumatol 44:224-8).

Giant cell arteritis (also known as Horton disease, cranial arteritis, and temporal arteritis) is the most common multisystem autoimmune disorder characterized by blood vessel inflammation in a group of multisystem autoimmune disorders known as systemic vasculitides. These diseases can often present with a systemic inflammatory syndrome and cytokine storm pathogenesis. T-cell cytokines produced by vasculitic lesions is typically multifunctional, including IL-2, IFN-y, IL-17, IL-21, and GM-CSF, supportive for a general defects in T cell regulation (Watanabe, R. et al., 2017, Joint Bone Spine 84:421-6). IL-2 is also up-regulated in the temporal arteries of patients with subclavian and aortic giant cell arteritis further supporting the pathogenic role of the cytokine (Weyand, C.M. et al., 1997, Arthritis Rheum 40:19-26). Cytokine profiling of patient biopsies determined that IL-9 overexpression and Th9 polarization predominated in arteries with transmural inflammation and small-vessel vasculitis. The tissue expression of IL-9, in addition to IL-17, was correlated with the intensity of the systemic inflammatory response (Ciccia, F. et al., 2015, Rheumatology 54:1596-604).

### T-Cell Based Immunotherapy Cytokine Storm Associated Disorders

As disclosed elsewhere herein, in some embodiments, the therapeutic compounds described herein may be used for inhibiting, ameliorating, reducing a severity of, treating, delaying the onset of, or preventing T-cell based immunotherapy cytokine storm associated disorders.

T-cell based therapies, including chimeric antigen receptor (CAR) T-cell therapy, have found a wide use against cancer, infectious disease, and the modulation of autoimmune disorders. Although CAR T-cell therapies have induced durable remission in hematological malignancies that are not responsive to standard therapies, early case reports have documented unexpected cytokine storm associated multi-system organ failure, neurotoxicity, and death. It has been established that uncontrolled CAR T-cell activation following engagement of tumor cell antigens can induce systemic inflammatory responses similar to those found in hemophagocytic lymphohistiocytosis and macrophage-activation syndrome (Bonifant, C.L. et al., 2016, Mol Ther Oncolytics 3:16011). The systemic inflammatory response induces high levels of IL-2, with other proinflammatory cytokines such as IFN-y, TNFα, IL-6, and MCP-1 resulting in pyrexia, hypotension, pulmonary edema, reduced renal perfusion, various cardiovascular toxicities, and immune effector cell-associated neurotoxicity syndrome (Bonifant, C.L. et al., 2016, Mol Ther Oncolytics 3:16011; Brudno, J.N. et al., 2016, Blood 127:3321-30; Lee, Y.G. et al., 2019, Nat Commun 10:2681). IL-2 is established as a key biomarker for severity CAR T-cell induced cytokine storm and immune effector cell-associated neurotoxicity syndrome (Wang, Z. et al., 2018, Biomark Res 6:4). Preclinical studies have also shown that CAR T-cells undergo stronger clonal expansion following stimulation and produce higher-immune-stimulatory cytokines such as IL-2 (Adusumilli, P.S. et al., 2014, Sci Transl Med 6:261ra151; Yang, Y. et al., 2017, Sci Transl Med 9:eaag1209).

T-cell bispecific antibody therapy is designed to redirect T-cell mediated lysis of malignant cancer cells via concurrently binding to a T-cell antigen, such as CD3 or CD28, and a tumor-specific antigen. However, the therapy can often result in severe cytokine storm pathogenic toxicity in the patient due to non-specific T-cell activation in both antigen-dependent and independent mechanisms and resultant systemic pro-inflammatory cytokine production (Link, B.K. et al., 1998, Int J Cancer 77:251-6; Belani, R. et al., 1995, J Hematother 4:395-402). IL-2 drives the proliferation of cytotoxic immune cells and release of pro-inflammatory cytokines, despite the induction of T-cells with regulatory phenotypes in both patient cohorts and preclinical models (Gogishvili, T. et al., 2009, PLoS One 4:e4643; Li, J. et al., 2019, Sci Transl Med 11:eaax8861; Suntharalingam, G. et al., 2006, N Engl J Med 355:1018-28).

### Pulmonary Infiltrate Cytokine Storm Associated Disorders

As disclosed elsewhere herein, in some embodiments, the therapeutic compounds described herein may be used for inhibiting, ameliorating, reducing a severity of, treating, delaying the onset of, or preventing pulmonary infiltrate cytokine storm associated disorders.

Acute respiratory distress syndrome (ARDS) is a disease pathology in response to various etiologies including pneumonia, trauma, infection, sepsis, pulmonary fibrosis, and interstitial lung disease (ILD) that can lead to a highly pathogenic cytokine storm environment. ARDS progresses through different phases, starting with alveolar-capillary damage, a proliferative phase characterized by improved lung function and healing, and a final fibrotic phase signaling the end of the acute disease process. The pulmonary epithelial and endothelial cellular damage is characterized by inflammation, apoptosis, necrosis, and increased alveolar-capillary permeability, which leads to the development of alveolar edema and proteinosis. Alveolar edema, in turn, reduces gas exchange, leading to hypoxemia. A variety of immune cells, including neutrophils, macrophages, and dendritic cells, have been shown to contribute to tissue injury in ARDS (Han, S.H. et al., 2015, J Immunol 194:855-60). Neutrophil influx into the lungs has been demonstrated to correlate with the severity of ARDS and may directly contribute to the pathogenesis of this disease (Williams, A.E. et al., 2014, Am J Physiol Lung Cell Mol Physiol 306:L217-30). Increased quantities of IL-2 and IL-15 in lungs and serum in patients suffering from ARDS is associated with worse disease prognosis (Agouridakis, P. et al., 2002, Eur J Clin Invest 32:862-7). Both IL-2 and IL-15 drive the proliferation of neutrophils and macrophages, and expression of pro-inflammatory cytokines IL-8, IL-6, IFN-y, TNFα, and GM-CSF, MCP-1 and other pro-fibrotic cytokines by immune and non-immune cells, promoting lung damage (Agostini, C. et al., 1996, J Immunol 157:910-8; Nakamura, R. et al., 2010, J Virol 84:5574-82; Wei, S. et al., 1993, J Immunol 150:1979-87; Welbourn, R. et al., 1990, Ann Surg 212:728-33; Welbourn, R. et al., 1991, Ann Surg 214:181-6). IL-2 prevents the apoptosis of neutrophils, propagating alveolar damage and vascular leak syndrome in ARDS (Lesur, O. et al., 2000, Crit Care Med 12:3814-22; Carey, P.D. et al., 1997, Surgery 122:918-26; Wei, S. et al., 1993, J Immunol 150:1979-87), and is upregulated in the serum of patients suffering from pulmonary fibrosis and is thought to contribute to the pathogenic ARDS immune response in the lung (Tsoutsou, P.G. et al., 2006, Respir Med 100:938-45). Additionally, in preclinical pulmonary fibrotic models, elevated expression of IL-9 was shown to have a direct pathogenic function likely leading to ARDS progression (van den Brûle, S. et al., 2007, Am J Respir Cell Mol Biol 37:202-9; Sugimoto, N. et al., 2019, Am J Respir Cell Mol Biol 60:232-43). In ILD associated ARDS, IL-15 is up-regulated in the lungs of patients and is proposed to contribute to an aberrant Th1-mediated chronic inflammatory response (Muro, S. et al., 2001, J Allergy Clin Immunol 108:970-5), whereas IL-2 directly activates alveolar macrophages leading to the activation and recruitment of innate immune cells to the site of inflammation and the release of pro-fibrotic cytokine factors (Gruss, H.J. et al., 1996, J Immunol 157:851-7; Hogaboam, C.M. et al., 1999, J Immunol 163:2193-201; Semenzato, G. et al., 2000, Allergy 55:1103-20). IL-15 is also produced by activated macrophages, which act to recruit natural killer cells and induce the production of IFN-y, which further leads to the potentiation of macrophage function and a systemic and fatal inflammatory response in pre-clinical models (Biber, J.L. et al., 2002, 216:31-42; Strengell, M. et al., 2003, J Immunol 170:5464-9).

Pneumonia refers to lung inflammation of the pulmonary parenchyma that is almost exclusively caused by infection of bacteria, fungi, parasites, or viruses. Specifically, infection causes bronchioles and alveoli to inflame and fill with fluid or pus which can subsequently become solid. This limits oxygen uptake and induces hypoxia. IL-2 is elevated in serum of patients suffering from community-acquired pneumonia (CAP) and is reliable predictor of in-hospital mortality and disease severity (Makarevich, A. et al., 2011, Eur Resp J 38:1474). CAP can result from numerous infectious agents, including Streptococcus pneumoniae, Haemophilus influenzae, Moraxella catarrhalis, Chlamydia pneumoniae, Mycoplasma pneumoniae, Legionella species, rhinovirus, coronavirus, and influenza. IL-2 has also been documented to be significantly increased in bronchoalveolar lavage fluid (BALF) of patients experiencing Mycoplasma pneumonia or pneumococcal pneumonia (Koh, Y.Y. et al., 2001, Pediatrics 107:E39). Elevated IL-2 is concurrent with an abundance of neutrophils and lymphocytes observed in BALF, cell types which are also implicated in IL-2 driven lung pathogenesis during acute respiratory distress syndrome. Preclinical studies evaluating influenza and parainfluenza induced pneumonias also determined that IL-2 is up-regulated in BALF fluid of infected animals (Carding, S.R. et al., 1993, J Exp Med 177:475-82; Sarawar, S.R. et al., 1993, Reg Immunol 5:142-50; Sarawar, S.R. et al., 1994, J Virol 68:3112-9; Mo, X.Y. et al., 1995, J Virol 69:1288-91). Both IL-9 and IL-15 are significantly up-regulated in the serum of patients suffering from CAP (Haugen, J. et al., 2015, PLoS One 10:e0138978). IL-15 expression is notably high in patients suffering from bacterial pneumonia (Liu, M. et al., 2018, Clin Respir J 12:974-85), and studies in animal models of Pneumocystis pneumonia and antibiotic-resistant Staphylococcus aureus pneumonia determined that neutralization of IL-9 enhanced pathogen clearance and attenuated pathogen-associated inflammation (Li, T. et al., 2018, Front Immunol 9:1118; Xu, W. et al., 2020, Acta Biochim Biophys Sin 52:133-40).

Acute interstitial pneumonia (also known as Hamman-Rich Syndrome) is a rare and fulminant form of interstitial lung disease, and has the histopathological appearance of diffuse alveolar damage. The disease generally affects healthy individuals without prior history of lung disease or smoking (Bruminhent, J. et al., 2011, Case Rep Med 2011:628743). In preclinical models, IL-2 expression has been shown to help drive the pathology of acute interstitial pneumonia by propagating natural killer cell cytotoxicity and up-regulating IFN-γ mediated gene expression leading to prolonged pathogenic inflammation in the lung (Okamoto, M. et al., 2002, Blood 99:1289-98; Segawa, S. et al., 2010, Clin Exp Immunol 160:394-402).

### Additional Methods

Several embodiments relate to the use of therapeutic antagonist peptides that selectively inhibit the activity of IL-15, either alone or in combination with the other IL-2 and IL-9 γc-cytokine family members, as a therapeutic agent for cytokine-release syndrome, and/or cytokine storm associated disorders. In some embodiments, custom derivative antagonist peptides that selectively inhibit IL-2, IL-15, IL-9, a combination of IL-2 and IL-15, a combination of IL-2 and IL-9, and/or a combination of IL-15 and IL-9 activities are used as a therapeutic agent for treating cytokine-release syndrome, and/or cytokine storm associated diseases. In some embodiments, the effect of custom derivative antagonist peptides that selectively inhibit a combination of IL-2 and IL-15, a combination of IL-2 and IL-9, and/or a combination of IL-15 and IL-9 can be additive or synergistic. Several embodiments relate to the use of BNZ-γ to treat cytokine-release syndrome, and/or cytokine storm associated disorders. Several embodiments relate to the use of SEQ ID NO: 1 to treat cytokine-release syndrome, and/or cytokine storm associated disorders.

Several embodiments relate to the use of therapeutic compounds, either alone or in combination, as a therapeutic agent for cytokine-release syndrome, and/or cytokine storm associated disorders. In some embodiments, the therapeutic compound is BNZ-y. In some embodiments, the therapeutic compound is SEQ ID NO: 1. In some embodiments, the therapeutic compound is a composite peptide derivative of SEQ ID NO: 1.

An additive effect is observed when the effect of a combination is equal to the sum of the effects of the individuals in the combination (e.g., the effect of a combination of two or more therapeutic compounds is equal to the sum of the effects of each therapeutic compound individually). A synergistic effect is observed when the effect of a combination is greater than the sum of the effects of the individuals in the combination (e.g., the effect of a combination of two or more therapeutic compounds is greater than the sum of the effects of each therapeutic compound individually). A synergistic effect is greater than an additive effect. Additive effect, synergistic effect, or both can occur in human patients, non-human patients, non-patient human volunteers, in vivo models, ex vivo models, in vitro models, etc.

In some embodiments, two or more therapeutic compounds disclosed herein can be used in combination. In some embodiments, two or more therapeutic compounds disclosed herein when used in combination yield an additive effect. In some embodiments, two or more therapeutic compounds disclosed herein when used in combination yield a synergistic effect. Synergistic effect can range from about >1 to about 100-fold. In some embodiments, the synergistic effect is about 2 to about 20-fold. In some embodiments, the synergistic effect is about 20 to about 100-fold. In some embodiments, the synergistic effect is from >1, 2, 3, 4, 5, 6, 7, 8, 9, 10, 20, 30, 40, 50, 60, 70, 80, 90, or 100-fold, or within a range defined by any two of the aforementioned values.

Another embodiment relates to the development of chemical compounds (non-peptide, non-protein) that have a spatial structure which resembles the 19-mer amino acid sequence I-K-E-F-L-Q-R-F-I-H-I-V-Q-S-I-I-N-T-S (SEQ ID NO: 1) and can fit into the pocket of the γc-subunit to structurally hinder the access of a IL-2, IL-9, or IL-15 γc-cytokine to the γc-subunit for binding. Some embodiments relate to the use of structurally similar chemical compounds as inhibitors of IL-2, IL-9, and IL-15 γc-cytokine activity. Such molecular mimicry strategy to further refine the development of synthetic compounds resembling in structure to existing biological peptide/proteins has been described (Orzaez et al., 2009, Chem Med Chem 4:146-60). Another embodiment relates to administration of chemical compounds (non-peptide, non-protein) that have a resembling 3D structure as the 19-mer amino acids sequence I-K-E-F-L-Q-R-F-I-H-I-V-Q-S-I-I-N-T-S (SEQ ID NO: 1) for inhibiting, ameliorating, reducing a severity of, treating, delaying the onset of, or preventing one or more cytokine storm associated disorders.

Several embodiments relate to the administration of a peptide of amino acid sequence I-K-E-F-L-Q-R-F-I-H-I-V-Q-S-I-I-N-T-S (SEQ ID NO: 1) for inhibiting, ameliorating, reducing a severity of, treating, delaying the onset of, or preventing one or more cytokine storm associated disorders. Another embodiment relates to the administration of derivative peptides of amino acid sequence I-K-E-F-L-Q-R-F-I-H-I-V-Q-S-I-I-N-T-S (SEQ ID NO: 1), wherein the amino acid sequence of the derivative peptide has similar physico-chemical properties as a peptide of the amino acid sequence I-K-E-F-L-Q-R-F-I-H-I-V-Q-S-I-I-N-T-S (SEQ ID NO: 1), but has distinct IL-2, IL-9, and IL-15 biological activity, for inhibiting, ameliorating, reducing a severity of, treating, delaying the onset of, or preventing one or more cytokine storm associated disorders. Another embodiment relates to administration of a peptide of amino acid sequence I-K-E-F-L-Q-R-F-I-H-I-V-Q-S-I-I-N-T-S (SEQ ID NO: 1) conjugated to the N- and C-termini or to the side residues of existing biological proteins/peptides into patients for inhibiting, ameliorating, reducing a severity of, treating, delaying the onset of, or preventing one or more cytokine storm associated disorders.

Several embodiments relate to administration of polyclonal and monoclonal antibodies raised against a peptide comprising of amino acid sequence I-K-E-F-L-Q-R-F-I-H-I-V-Q-S-I-I-N-T-S (SEQ ID NO: 1) into patients as an immunogen for inhibiting, ameliorating, reducing a severity of, treating, delaying the onset of, or preventing one or more cytokine storm associated disorders. Another embodiment relates to administration of polyclonal and monoclonal antibodies that were raised against derivative peptides of amino acid sequence I-K-E-F-L-Q-R-F-I-H-I-V-Q-S-I-I-N-T-S (SEQ ID NO: 1), wherein the amino acid sequence of the derivative peptide has similar physico-chemical properties as a peptide of the amino acid sequence I-K-E-F-L-Q-R-F-I-H-I-V-Q-S-I-I-N-T-S (SEQ ID NO: 1), but has distinct IL-2, IL-9, or IL-15 biological activity, into patients as an immunogen for inhibiting, ameliorating, reducing a severity of, treating, delaying the onset of, or preventing one or more cytokine storm associated disorders.

### Administration of Therapeutic Compounds

The present embodiments also encompass the use of one or more therapeutic compounds selected from the group consisting of a γc-cytokine antagonist peptide, a γc-cytokine antagonist peptide derivative, or a combination thereof for the manufacture of a medicament for inhibiting, ameliorating, reducing a severity of, treating, delaying the onset of, or preventing one or more cytokine storm associated disorders. The present embodiments also encompass a pharmaceutical composition that includes one or more therapeutic compounds in combination with a pharmaceutically acceptable carrier. The pharmaceutical composition can include a pharmaceutically acceptable carrier and a non-toxic therapeutically effective amount of therapeutic compounds, or other compositions of the present embodiments.

The present embodiments provide methods of using pharmaceutical compositions comprising an effective amount of therapeutic compounds in a suitable diluent or carrier. A therapeutic compound of the present embodiments can be formulated according to known methods used to prepare pharmaceutically useful compositions. A therapeutic compound can be combined in admixture, either as the sole active material or with other known active materials, with pharmaceutically suitable diluents (e.g., phosphate, acetate, Tris-HCl), preservatives (e.g., thimerosal, benzyl alcohol, parabens), emulsifying compounds, solubilizers, adjuvants, and/or carriers such as bovine serum albumin.

In some embodiments, one or more compositions and kits comprising one or more of the therapeutic compounds disclosed herein are contemplated. In some embodiments, one or more compositions and kits are used for preventing and/or treating one or more diseases. In some embodiments, one or more compositions and kits are used for inhibiting, ameliorating, reducing a severity of, treating, delaying the onset of, or preventing one or more cytokine storm associated disorder.

In some embodiments, the one or more compositions and kits comprising one or more of the therapeutic compounds are administered to a subject in need thereof via any of the routes of administration provided herein. In some embodiments, the one or more compositions and kits comprises one or more of the therapeutic compounds at a therapeutically effective amount to modulate the signaling of one or more γc-cytokines selected from the group consisting of IL-2, IL-9, and IL-15. In some embodiments, the one or more compositions and kits comprises one or more of the therapeutic compounds at a therapeutically effective amount to prevent and/or treat one or more diseases. In some embodiments, the one or more compositions and kits comprising one or more of the therapeutic compounds additionally comprise one or more pharmaceutically acceptable carriers, diluents, excipients or combinations thereof.

In some embodiments, one or more therapeutic compounds in the one or more compositions and kits are formulated as suitable for administration to a subject for preventing and/or treating one or more diseases. In some embodiments, one or more therapeutic compounds in the one or more compositions and kits are formulated as suitable for administration to a subject for preventing and/or treating a cytokine storm associated disorder.

In some embodiments, one or more therapeutic compounds selected from the group consisting of SEQ ID NO: 1 and a derivative of SEQ ID NO: 1 in the one or more compositions and kits are formulated as suitable for administration to a subject for preventing and/or treating one or more diseases. In some embodiments, one or more composite peptides selected from the group consisting of SEQ ID NO: 1 and a derivative of SEQ ID NO: 1 in the one or more compositions and kits are formulated as suitable for administration to a subject for inhibiting, ameliorating, reducing a severity of, treating, delaying the onset of, or preventing one or more cytokine storm associated disorder.

The terms “disease,” “disorder,” and “biological condition” can be used interchangeably when referring to “inhibiting, ameliorating, reducing a severity of, treating, delaying the onset of, or preventing one or more diseases” provided in accordance with the present embodiments.

In some embodiments, the one or more derivatives of the one or more composite peptides comprise amino acid sequences that shares about 60% to about 99% identity with the one or more composite peptides. In some embodiments, the one or more derivatives of the one or more composite peptides comprise amino acid sequences that shares 60-70%, 70-80%, 80%, 90%, 95%, 97%, 98%, 99% or 99.8% identity with the one or more composite peptides, or within a range defined by any two of the aforementioned values.

In some embodiments, one or more cytokine storm associated disorder is selected from the group consisting of cytokine release syndrome, cytokine storm, multiple organ dysfunction syndrome, systemic inflammatory response syndrome, sepsis, septic shock, graft-versus-host disease, haploidentical donor transplantation, sarcoidosis, hemophagocytic lymphohistiocytosis, vascular leak syndrome, systemic capillary leak syndrome, Stevens-Johnson syndrome, toxic epidermal necrolysis, asthmatic allergic lung inflammation, rhinosinusitis, viral infection, coronavirus infection, multi-system inflammatory syndrome in children (MIS-C) associated with COVID-19 (or another coronavirus disease), viral hemorrhagic fever, influenza viral infection, hantaviral infection, Epstein-Barr viral infection, HIV/HCV coinfection liver fibrosis, fungal infection, pulmonary Aspergillosis, bacterial infection, toxic shock syndrome, lyme neuroborreliosis, lyme disease, autoimmune disease, juvenile idiopathic arthritis, Still’s disease, macrophage activation syndrome, Sjögren’s syndrome, systemic sclerosis, inflammatory myopathies, systemic vasculitides, giant cell arteritis, Horton disease, cranial arteritis, temporal arteritis, T-cell based immunotherapy induced cytokine storm, chimeric antigen receptor T-cell therapy induced cytokine storm, immune effector cell-associated neurotoxicity syndrome, T-cell bispecific antibody therapy induced cytokine storm, pulmonary infiltrate, adult respiratory distress syndrome, interstitial lung disease, pneumonia, community acquired pneumonia, and acute interstitial pneumonia.

Suitable carriers and their formulations are described in Remington’s Pharmaceutical Sciences, 16^{th} ed. 1980 Mack Publishing CO, and Overview of Antibody Drug Delivery (Awwad et al., 2018, Pharmaceutics 10:83). Additionally, such compositions can contain a therapeutic compound complexed with polyethylene glycol (PEG), metal ions, or incorporated into polymeric compounds such as polyacetic acid, polyglycolic acid, hydrogels etc., or incorporated into liposomes, microemulsions, micelles, unilamellar or multilamellar vesicles, erythrocyte ghosts, or spheroblasts. Such compositions will influence the physical state, solubility, stability, rate of in vivo release, and rate of in vivo clearance of a therapeutic compound. A therapeutic compound can be conjugated to antibodies against cell-specific antigens, receptors, ligands, or coupled to ligands for tissue-specific receptors.

Methods of administrating therapeutic compounds of the present embodiments may be selected as appropriate, depending on factors, such as the type of diseases, the condition of subjects, and/or the site to be targeted. The therapeutic compounds can be administered topically, orally, parenterally, rectally, or by inhalation. Topical administration of therapeutic compounds can be achieved through formulation into lotions, liniments (balms), solutions, ointments, creams, pastes, gels, or other suitable topical delivery systems as appropriate (Gupta et al., 2016, Indo Amer J Pharm Res 6:6353-69). Topical formulation components can include emollient and/or stiffening agents such as cetyl alcohol, cetyl ester wax, carnauba wax, lanolin, lanolin alcohols, paraffin, petrolatum, polyethylene glycol, stearic acid, stearyl alcohol, white or yellow wax; emulsifying and/or solubilizing agents such as polysorbate 20, polysorbate 80, polysorbate 60, poloxamer, sorbitan monostearate, sorbitan monooleate, sodium lauryl sulfate, propylene glycol monostearate; humectants such as glycerin, propylene glycol, polyethylene glycol; thickening/gelling agents such as carbomer, methyl cellulose, sodium carboxyl methyl cellulose, carrageenan, colloidal silicon dioxide, guar gum, hydroxypropyl cellulose, hydroxypropyl methyl cellulose, gelatin, polyethylene oxide, alginic acid, sodium alginate, fumed silica; preservative agents such as benzoic acid, propyl paraben, methyl paraben, imidurea, sorbic acid, potassium sorbate, benzalkonium chloride, phenyl mercuric acetate, chlorobutanol, phenoxyethanol; permeation enhancing agents such as propylene glycol, ethanol, isopropyl alcohol, oleic acid, polyethylene glycol; antioxidant agents such as butylated hydroxyanisole, butylated hydroxytoluene; buffering agents such as citric acid, phosphoric acid, sodium hydroxide, monobasic sodium phosphate; and vehicle agents such as purified water, propylene glycol, hexylene glycol, oleyl alcohol, propylene carbonate, and mineral oil (Chang et al., 2013, AAPS J 15:41-52). Oral formulation components can include fatty acids and derivatives such as lauric acid, caprylic acid, oleic acid; bile salts such as sodium cholate, sodium deoxycholate, sodium taurodeoxycholate, sodium glycocholate; chelators such as citric acid, sodium salicylate; alkylglycoside containing polymers, cationic polymers, anionic polymers, and nanoparticles; and surfactants such as sodium dodecyl sulfate, sodium laurate dodecylmaltoside, polaxamer, sodium myristate, sodium laurylsulfate, quillayasaponin, and sucrose palmitate (Liu et al., 2018, Expert Opin Drug Del 15:223-33; Aguirre et al., 2016, Adv Drug Deliv Rev 106:223-41). The term “parenteral” includes subcutaneous injections, intravenous, intramuscular, intraperitoneal, intracisternal injection, or infusion techniques. These compositions will typically include an effective amount of a therapeutic compound, alone or in combination with an effective amount of any other active material. Several non-limiting routes of administrations are possible including parenteral, subcutaneous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

The one or more therapeutic compounds disclosed herein can be administered at any dose, via any of the routes of administration, and at any frequency of administration as determined by one of ordinary skill in the art based on various parameters. Non-limiting examples of which include the condition being treated, the severity of the condition, patient compliance, efficacy of treatment, side effects, etc.

The amount of the therapeutic compound contained in pharmaceutical compositions of the present embodiments, dosage form of the pharmaceutical compositions, frequency of administration, and the like may be selected as appropriate, depending on factors, such as the type of diseases, the condition of subjects, and/or the site to be targeted. Such dosages and desired drug concentrations contained in the compositions may vary affected by many parameters, including the intended use, patient’s body weight and age, and the route of administration. Pilot studies will first be conducted using animal studies and the scaling to human administration will be performed according to art-accepted practice.

In one embodiment, host cells that have been genetically modified with a polynucleotide encoding at least one therapeutic compound are administered to a subject for inhibiting, ameliorating, reducing a severity of, treating, delaying the onset of, or preventing one or more cytokine storm associated disorder. The polynucleotide is expressed by the host cells, thereby producing the therapeutic compound within the subject. Preferably, the host cells are allogeneic or autogeneic to the subject.

In a further aspect, the one or more therapeutic compounds selected from the group consisting of a γc-cytokine antagonist peptide, a γc-cytokine antagonist peptide derivative, or a combination thereof can be used in combination with other therapies, for example, therapies inhibiting cancer cell proliferation and growth, and/or with other immunomodulators, antibiotics, antivirals, steroids, anti-bacterial compounds, anti-fungal compounds, and T-cell based immunotherapies. The phrase “combination therapy” embraces the administration of the one or more therapeutic compounds selected from the group consisting of a γc-cytokine antagonist peptide, a γc-cytokine antagonist peptide derivative, or a combination thereof and one or more additional therapeutic agent as part of a specific treatment regimen intended to provide a beneficial effect from the co-action of these therapeutic agents. Administration of these therapeutic agents in combination typically is carried out over a defined time period (usually minutes, hours, days or weeks depending upon the combination selected).

A combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by an appropriate route including, but not limited to, oral routes, intravenous routes, intramuscular routes, and direct absorption through mucous membrane tissues. The therapeutic agents can be administered by the same route or by different routes. The sequence in which the therapeutic agents are administered is not narrowly critical.

Combination therapy also can embrace the administration of the therapeutic agents as described above in further combination with other biologically active ingredients (such as, but not limited to, a second and different therapeutic agent) and non-drug therapies (such as, but not limited to, surgery, radiation treatment, or natural products and ointments). Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporarily removed from the administration of the therapeutic agents, perhaps by days or even weeks.

In certain embodiments, the one or more therapeutic compounds selected from the group consisting of a γc-cytokine antagonist peptide, a γc-cytokine antagonist peptide derivative, or a combination thereof can be administered in combination with at least one antiproliferative agent selected from the group consisting of chemotherapeutic agent, an antimetabolite, an anti-tumorgenic agent, an antimitotic agent, an antiviral agent, an immunomodulating agent, an antibiotic agent, an anti-bacterial agent, an anti-fungal agent, T-cell based immunotherapies, an antineoplastic agent, an immunotherapeutic agent, and a radiotherapeutic agent.

In certain embodiments, the one or more therapeutic compounds selected from the group consisting of a γc-cytokine antagonist peptide, a γc-cytokine antagonist peptide derivative, or a combination thereof can be administered in combination with at least one anti-inflammatory agent selected from the group consisting of steroids, corticosteroids, and nonsteroidal anti-inflammatory drugs.

Also provided are kits for performing any of the above methods. Kits may include the one or more therapeutic compounds selected from the group consisting of a γc-cytokine antagonist peptide, a γc-cytokine antagonist peptide derivative, or a combination thereof according to the present embodiments. In some embodiments, the kit may include instructions. Instructions may be in written or pictograph form, or may be on recorded media including audio tape, audio CD, video tape, DVD, CD-ROM, or the like. The kits may comprise packaging.

### Additional Embodiments

In some embodiments of the method, the composite peptide comprises the amino acid sequence I-K-E-F-L-Q-R-F-I-H-I-V-Q-S-I-I-N-T-S (SEQ ID NO: 1) (BNZ-y). In some embodiments of the method, the composite peptide derivative shares at least about 60% identity with a peptide of SEQ ID NO: 1. In some embodiments of the method, the composite peptide derivative shares at least about 90% identity with a peptide of SEQ ID NO: 1. In some embodiments of the method, the composite peptide derivative shares at least about 95% identity with a peptide of SEQ ID NO: 1. In some embodiments of the method, the composite peptide and the composite peptide derivative have similar physico-chemical properties but distinct IL-2, IL-9, or IL-15 biological activities.

In some embodiments of the method, the composite peptide or composite peptide derivative inhibits the activity of one or more γc-cytokines. In some embodiments of the method, the one or more γc-cytokines are selected from the group consisting of IL-2, IL-9, and IL-15. In some embodiments of the method, the composite peptide or composite peptide derivative inhibits the activity of IL-2, IL-15 and IL-9. In some embodiments of the method, the composite peptide or composite peptide derivative inhibits the activity of IL-2 and IL-15. In some embodiments of the method, the composite peptide or composite peptide derivative inhibits the activity of IL-15 and IL-9. In some embodiments of the method, the composite peptide or composite peptide derivative inhibits the activity of IL-2 and IL-9.

In some embodiments, the composite peptide or composite peptide derivative comprises a signal peptide. In some embodiments, the composite peptide or composite peptide derivative is further conjugated to one or more additional moieties at the N terminus, C terminus or a side residue of the composite peptide or composite peptide derivative. In some embodiments of the composite peptide or composite peptide derivative, the one or more additional moieties are selected from the group consisting of bovine serum albumin (BSA), albumin, Keyhole Limpet Hemocyanin (KLH), Fc region of IgG, a biological protein that functions as scaffold, an antibody against a cell-specific antigen, a receptor, a ligand, a metal ion, and Poly Ethylene Glycol (PEG).

In some embodiments, the composite peptide or composite peptide derivative comprises at least two alpha-alkenyl substituted amino acids, and wherein the at least two alpha-alkenyl substituted amino acids are linked via at least one intra-peptide hydrocarbon linker element is provided. In some embodiments of the composite peptide, the at least two alpha-alkenyl substituted amino acids are linked to form the at least one intra-peptide hydrocarbon linker element by ring closing metathesis, wherein the ring closing metathesis is catalyzed by Grubb’s catalyst.

In some embodiments, an amino acid in the composite peptide is selected from the group consisting of natural amino acids, non-natural amino acids, (D) stereochemical configuration amino acids, (L) stereochemical configuration amino acids, (R) stereochemical configuration amino acids and (S) stereochemical configuration amino acids, and wherein the at least two alpha-alkenyl substituted amino acids are selected from S-pentenylalanine (CAS: 288617-73-2; S5Ala) and R-octenylalanine (CAS: 945212-26-0; R8Ala).

In some embodiments of the composite peptide, the at least two alpha-alkenyl substituted amino acids linked by the at least one intra-peptide hydrocarbon are separated by n-2 amino acids, wherein n represents the number of amino acids encompassed by the intra-peptide linkage.

In some embodiments of the composite peptide, when the at least two alpha-alkenyl substituted amino acids linked by the at least one intra-peptide hydrocarbon are separated by three amino acids, the at least one intra-peptide hydrocarbon linker element spans a single α-helical turn of the composite peptide.

In some embodiments of the composite peptide, when the composite peptide comprises one or more non-contiguous single α-helical turns, the amino acid positions that correlate with a single α-helical turn of the composite peptide correspond to amino acid positions i and i+4 of the composite peptide, where i is the first amino acid position of the single α-helical turn and i+4 is the last amino acid position of the single α-helical turn, and wherein amino acid positions i and i+4 comprise alpha-alkenyl substituted amino acids. In some embodiments of the composite peptide, when the alpha-alkenyl substituted amino acid at position i is S5Ala, the alpha-alkenyl substituted amino acid at position i+4 is also S5Ala, the hydrocarbon linker element formed by the ring-closing metathesis is represented by Formula 1.

In some embodiments of the composite peptide, when the at least two alpha-alkenyl substituted amino acids linked by the at least one intra-peptide hydrocarbon are separated by six residues, the at least one intra-peptide hydrocarbon linker element spans a double α-helical turn of the composite peptide.

In some embodiments of the composite peptide, when the composite peptide comprises one or more non-contiguous double α-helical turns, the amino acid positions that correlate with a double α-helical turn of the composite peptide correspond to amino acid positions i and i+7 of the composite peptide, where i is the first amino acid position of the double α-helical turn and i+7 is the last amino acid position of the double α-helical turn, and wherein amino acid positions i and i+7 comprise alpha-alkenyl substituted amino acids. In some embodiments of the composite peptide, when the alpha-alkenyl substituted amino acid at position i is R8Ala, the alpha-alkenyl substituted amino acid at position i+7 is S5Ala, the hydrocarbon linker element formed by the ring-closing metathesis is represented by Formula 2.

In some embodiments, the composite peptide comprises amino acid sequences of at least two interleukin (IL) protein gamma-c-box D-helix regions, wherein the composite peptide comprises the amino acid sequence I-K-E-F-L-Q-R-F-I-H-I-V-Q-S-I-I-N-T-S (SEQ ID NO: 1), and wherein the composite peptide comprises at least two alpha-alkenyl substituted amino acids, and wherein the at least two alpha-alkenyl substituted amino acids are linked via at least one intra-peptide hydrocarbon linker element.

In some embodiments of the composite peptide, the one or more carbon-carbon double bonds present in the intra-peptide hydrocarbon linker are utilized for one or more organic chemical reactions to add one or more additional chemical functionalities. In some embodiments of the composite peptide, the one or more organic chemical reactions comprises an alkene reaction. In some embodiments of the composite peptide, the alkene reaction is selected from the group consisting of hydroboration, oxymercuration, hydration, chlorination, bromination, addition of HF, HBr, HC1 or HI, dihydroxylation, epoxidation, hydrogenation, and cyclopropanation. In some embodiments of the composite peptide, one or more additional chemical functionalities can be added subsequent to the alkene reaction wherein the one or more additional chemical functionalities comprise a covalent addition of one or more chemical group substituents, wherein the covalent addition of one or more chemical group substituents comprises nucleophilic reactions with epoxide and hydroxyl groups. In some embodiments of the composite peptide, the one or more additional chemical functionalities are selected from the group consisting of biotin, radioisotopes, therapeutic agents, rapamycin, vinblastine, taxol, non-protein fluorescent chemical groups, FITC, hydrazide, rhodamine, maleimide, protein fluorescent groups, GFP, YFP, and mCherry.

In some embodiments, a pharmaceutical composition is provided. In some embodiments, the pharmaceutical composition comprises a therapeutically effective amount of a peptide conjugate or a derivative thereof, and a pharmaceutically acceptable carrier, diluent, excipient or combination thereof, wherein the peptide conjugate or the derivative thereof modulates the activity of two or more γc-cytokines selected from the group consisting of IL-2, IL-9, and IL-15, wherein the peptide conjugate comprises the amino acid sequence I-K-E-F-L-Q-R-F-I-H-I-V-Q-S-I-I-N-T-S (SEQ ID NO: 1), and wherein the derivative thereof comprises a peptide sequence sharing at least 90% identity with the amino acid sequence of SEQ ID NO: 1.

In some embodiments of the pharmaceutical composition, the peptide conjugate or the derivative thereof inhibits the activity of two or more γc-cytokines selected from the group consisting of IL-2, IL-9, and IL-15. In some embodiments of the pharmaceutical composition, the peptide conjugate or the derivative thereof further comprises an additional conjugate at the N termini, C termini or a side residues thereof.

In some embodiments of the pharmaceutical composition, the peptide conjugate or the derivative thereof further comprises a signal peptide. In some embodiments, the pharmaceutical composition further comprises a protein that stabilizes the structure of the peptide conjugate or the derivative thereof and improves its biological activity, wherein the protein is selected from the group consisting of bovine serum albumin (BSA), albumin, Fc region of immunoglobulin G (IgG), biological proteins that function as scaffold, Poly Ethylene Glycol (PEG), and derivatives thereof. In some embodiments of the pharmaceutical composition, the derivative thereof comprises a peptide sequence sharing at least 95% identity with the amino acid sequence of SEQ ID NO: 1.

In some embodiments, a method of treating a cytokine storm associated disease is provided. In some embodiments, the method comprises administering a pharmaceutical composition provided herein to a subject in need thereof, wherein the cytokine storm associated disease is selected from the group consisting of cytokine release syndrome, cytokine storm, multiple organ dysfunction syndrome, systemic inflammatory response syndrome, sepsis, septic shock, graft-versus-host disease, haploidentical donor transplantation, sarcoidosis, hemophagocytic lymphohistiocytosis, vascular leak syndrome, systemic capillary leak syndrome, Stevens-Johnson syndrome, toxic epidermal necrolysis, asthmatic allergic lung inflammation, rhinosinusitis, viral infection, coronavirus infection, multi-system inflammatory syndrome in children (MIS-C) associated with COVID-19 (or other coronavirus diseases), viral hemorrhagic fever, influenza viral infection, hantaviral infection, Epstein-Barr viral infection, HIV/HCV coinfection liver fibrosis, fungal infection, pulmonary Aspergillosis, bacterial infection, toxic shock syndrome, lyme neuroborreliosis, lyme disease, autoimmune disease, juvenile idiopathic arthritis, Still’s disease, macrophage activation syndrome, Sjögren’s syndrome, systemic sclerosis, inflammatory myopathies, systemic vasculitides, giant cell arteritis, Horton disease, cranial arteritis, temporal arteritis, T-cell based immunotherapy induced cytokine storm, chimeric antigen receptor T-cell therapy induced cytokine storm, immune effector cell-associated neurotoxicity syndrome, T-cell bispecific antibody therapy induced cytokine storm, pulmonary infiltrate, adult respiratory distress syndrome, interstitial lung disease, pneumonia, community acquired pneumonia, and acute interstitial pneumonia.

In some embodiments, a kit for treating a cytokine storm associated disease in a patient is provided.

In some embodiments, the kit comprises a pharmaceutical composition, wherein the pharmaceutical composition comprises a therapeutically effective amount of a peptide conjugate, or a derivative thereof, and a pharmaceutically acceptable carrier, diluent, excipient or combination thereof, wherein the peptide conjugate or the derivative thereof modulates the activity of two or more γc-cytokines selected from the group consisting of IL-2, IL-9, and IL-15, wherein the peptide conjugate comprises the amino acid sequence I-K-E-F-L-Q-R-F-I-H-I-V-Q-S-I-I-N-T-S (SEQ ID NO: 1), and wherein the derivative thereof comprises a peptide sequence sharing at least 90% identity with the amino acid sequence of SEQ ID NO: 1.

In some embodiments of the kit, the condition is one or more of cytokine release syndrome, cytokine storm, multiple organ dysfunction syndrome, systemic inflammatory response syndrome, sepsis, septic shock, graft-versus-host disease, haploidentical donor transplantation, sarcoidosis, hemophagocytic lymphohistiocytosis, vascular leak syndrome, systemic capillary leak syndrome, Stevens-Johnson syndrome, toxic epidermal necrolysis, asthmatic allergic lung inflammation, rhinosinusitis, viral infection, coronavirus infection, multi-system inflammatory syndrome in children (MIS-C) associated with COVID-19 (or other coronavirus diseases), viral hemorrhagic fever, influenza viral infection, hantaviral infection, Epstein-Barr viral infection, HIV/HCV coinfection liver fibrosis, fungal infection, pulmonary Aspergillosis, bacterial infection, toxic shock syndrome, lyme neuroborreliosis, lyme disease, autoimmune disease, juvenile idiopathic arthritis, Still’s disease, macrophage activation syndrome, Sjögren’s syndrome, systemic sclerosis, inflammatory myopathies, systemic vasculitides, giant cell arteritis, Horton disease, cranial arteritis, temporal arteritis, T-cell based immunotherapy induced cytokine storm, chimeric antigen receptor T-cell therapy induced cytokine storm, immune effector cell-associated neurotoxicity syndrome, T-cell bispecific antibody therapy induced cytokine storm, pulmonary infiltrate, adult respiratory distress syndrome, interstitial lung disease, pneumonia, community acquired pneumonia, and acute interstitial pneumonia.

### Definitions

As used herein, the term “patient” or “subject” refers to the recipient of any of the embodiments of the composite peptides disclosed herein and includes all organisms within the kingdom animalia. In some embodiments, any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, etc.) are included. In preferred embodiments, the animal is within the family of mammals, such as humans, bovine, ovine, porcine, feline, buffalo, canine, goat, equine, donkey, deer, and primates. The most preferred animal is human. In some embodiments, the patient is a male or a female.

As used herein, the term “treat” or any variation thereof (e.g.,, treatment, treating, etc.), refers to any treatment of a patient diagnosed with a biological condition, such as cytokine release syndrome, cytokine storm, multiple organ dysfunction syndrome, systemic inflammatory response syndrome, sepsis, septic shock, graft-versus-host disease, haploidentical donor transplantation, sarcoidosis, hemophagocytic lymphohistiocytosis, vascular leak syndrome, systemic capillary leak syndrome, Stevens-Johnson syndrome, toxic epidermal necrolysis, asthmatic allergic lung inflammation, rhinosinusitis, viral infection, coronavirus infection, multi-system inflammatory syndrome in children (MIS-C) associated with COVID-19 (or other coronavirus diseases), viral hemorrhagic fever, influenza viral infection, hantaviral infection, Epstein-Barr viral infection, HIV/HCV coinfection liver fibrosis, fungal infection, pulmonary Aspergillosis, bacterial infection, toxic shock syndrome, lyme neuroborreliosis, lyme disease, autoimmune disease, juvenile idiopathic arthritis, Still’s disease, macrophage activation syndrome, Sjögren’s syndrome, systemic sclerosis, inflammatory myopathies, systemic vasculitides, giant cell arteritis, Horton disease, cranial arteritis, temporal arteritis, T-cell based immunotherapy induced cytokine storm, chimeric antigen receptor T-cell therapy induced cytokine storm, immune effector cell-associated neurotoxicity syndrome, T-cell bispecific antibody therapy induced cytokine storm, pulmonary infiltrate, adult respiratory distress syndrome, interstitial lung disease, pneumonia, community acquired pneumonia, and acute interstitial pneumonia.

The term treat, as used herein, includes: (i) preventing or delaying the presentation of symptoms associated with the biological condition of interest in an at-risk patient who has yet to display symptoms associated with the biological condition; (ii) ameliorating the symptoms associated with the biological condition of interest in a patient diagnosed with the biological condition; (iii) preventing, delaying, or ameliorating the presentation of symptoms associated with complications, conditions, or diseases associated with the biological condition of interest in either an at-risk patient or a patient diagnosed with the biological condition; (iv) slowing, delaying or halting the progression of the biological condition; and/or (v) preventing, delaying, slowing, halting or ameliorating the cellular events of inflammation; and/or (vi) preventing, delaying, slowing, halting or ameliorating the histological abnormalities and/or other clinical measurements of the biological condition.

The term “symptom(s)” as used herein, refers to common signs or indications that a patient is suffering from a specific condition or disease.

The term “effective amount,” as used herein, refers to the amount necessary to elicit the desired biological response. In accordance with the present embodiments, an effective amount of a γc-antagonist is the amount necessary to provide an observable effect in at least one biological factor for use in treating a biological condition.

“Recombinant DNA technology” or “recombinant” refers to the use of techniques and processes for producing specific polypeptides from microbial (e.g., bacterial, yeast), invertebrate (insect), mammalian cells or organisms (e.g., transgenic animals or plants) that have been transformed or transfected with cloned or synthetic DNA sequences to enable biosynthesis of heterologous peptides. Native glycosylation pattern will only be achieved with mammalian cell expression system. Prokaryotic expression systems lack the ability to add glycosylation to the synthesized proteins. Yeast and insect cells provide a unique glycosylation pattern that may be different from the native pattern.

A “nucleotide sequence” refers to a polynucleotide in the form of a separate fragment or as a component of a larger DNA construct that has been derived from DNA or RNA isolated at least once in substantially pure form, free of contaminating endogenous materials and in a quantity or concentration enabling identification, manipulation, and recovery of its component nucleotide sequences by standard molecular biology methods (as outlined in Current Protocols in Molecular Biology).

“Recombinant expression vector” refers to a plasmid comprising a transcriptional unit containing an assembly of (1) a genetic clement or elements that have a regulatory role in gene expression including promoters and enhances, (2) a structure or coding sequence that encodes the polypeptide according to the present embodiments, and (3) appropriate transcription and translation initiation sequence and, if desired, termination sequences. Structural elements intended for use in yeast and mammalian system preferably include a signal sequence enabling extracellular secretion of translated polypeptides by yeast or mammalian host cells.

“Recombinant microbial expression system” refers to a substantially homogenous monoculture of suitable hot microorganisms, for example, bacteria such as E. coli, or yeast such as S. cerevisiae, that have stably integrated a recombinant transcriptional unit into chromosomal DNA or carry the recombinant transcriptional unit as a component of a residual plasmid. Generally, host cells constituting a recombinant microbial expression system are the progeny of a single ancestral transformed cell. Recombinant microbial expression systems will express heterologous polypeptides upon induction of the regulatory elements linked to a structural nucleotide sequence to be expressed.

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this invention has been disclosed in the context of certain embodiments and examples, those skilled in the art will understand that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure.

It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes or embodiments of the disclosed invention. Thus, it is intended that the scope of the present invention herein disclosed should not be limited by the particular disclosed embodiments described above.

It should be understood, however, that this detailed description, while indicating preferred embodiments of the invention, is given by way of illustration only, since various changes and modifications within the spirit and scope of the invention will become apparent to those skilled in the art.

### EXAMPLES

The following Examples are presented for the purposes of illustration and should not be construed as limitations.

### Example 1 - Method for Assessing the Inhibitory Activity of γc-Antagonist Peptide

The capacity of any custom derivative peptide prepared according to the present embodiments for inhibiting the action of one γc-cytokine family member is determined using mammalian cellular assays to measure their proliferative response to the γc-cytokine family member.

For each of the six γc-cytokines, indicator cell lines: NK92, a human NK cell line NK92 available by American Type Culture Collection (ATCC) (catalog # CRL-2407), CTLL-2, a murine CD8 T cells line available from ATCC, and PT-18, a murine mast cell line and its subclone PT-18β, is transfected with human IL-2Rβ gene to make the cells responsive to IL-2 and IL-15 (Tagaya et al., 1996, EMBO J. 15:4928-39), and is used to quantitatively determine the γc-cytokine’s growth-promoting activity (See Current protocols in Immunology from Wiley and Sons for a methodological reference). The indicator cells demonstrate semi-linear dose-dependent response when measured by a colorimetric WST-1 assay over a range of concentrations (See Clontech PT3946-1 and associated user manual, incorporated herein by reference, for a detailed description of the reagents and methods).

Once the appropriate doses of the cytokine that yield the 50% and 95% maximum response from the indicator cell line is determined, various concentrations (ranging from 1 pM to 10 µM) of the purified or synthesized custom derivative peptide is added to each well containing the cytokine and indicator cells. The reduction in light absorbance at 450 nm is used as an indicator of inhibition of cytokine-stimulated cellular proliferation. Typically, the cells are stimulated by the cytokines such that the absorbance of the well containing indicator cell line and the cytokine is between 2.0 and 3.0, which is reduced to a range of 0.1 to 0.5 by the addition of inhibitory peptides.

### Example 2 - The Selective Inhibition of the Growth-Promoting Activities of Certain γc-Cytokines by BNZ-γ

Using PT-18β cells as described above, the ability of the BNZ-γpeptide to specifically inhibit the growth-promoting activity of select γc-cytokines was determined (FIG. 3A). IL-3, a non-γc-cytokine that supports the growth of PT-18β cells, was used as a negative control. Briefly, PT-18β cells were incubated either with two different dilutions of BNZ-γ peptide produced by HEK293T cells (1:20 or 1:60 dilution of the original supernatant of HEK293T cells transfected with a BNZ-γ expression construct) or without BNZ-γ peptide in the presence of IL-3, IL-9, IL-15, or IL-4 (1 nM of each cytokine in the culture).

The growth-responses of the cells were determined 2 days after the introduction of BNZ-γpeptide and the cytokine using the WST-1 assay. The growth-promoting activity of IL-3 (a non γc-cytokine) was not inhibited by BNZ-γ. In contrast, the activity of IL-15 and IL-9 were significantly (p<0.01 Student’s T test) reduced by the BNZ-γpeptide. Cellular proliferation stimulated by IL-4, another γc-cytokine, was not affected by the by the addition of BNZ-γpeptide. Results for IL-3, IL-9, IL-15, and IL-4 are shown at FIG. 3A.

In a similar assay, the murine cell line CTTL2 was used. In this assay the cells were cultured with 0.5 nM of recombinant IL-2 in RPMI 10% fetal Calf Serum. To set up the proliferation assay, cells were washed from the cytokines 3 times. Cells were seeded at 1 × 10(5) cells per well of a 96-well plate with final concentration of 50 pM of IL-2 or IL-15. Various concentration of BNZ-γ peptide (0.1, 1, and 10 µM) was added to each well. Cells were cultured for 20 hours and in the last 4 hours, ³H-thymidine was added to the plates. Cells were harvested and radioactivity measured to determine cell proliferation levels. The data are shown in FIG. 3B.

### Example 3 - Method for Measuring Inhibition γc-Cytokine Activity by Assaying 3H-Thymidine Incorporation of as a Marker of Cellular Proliferation

Inhibition of γc-cytokine-induced proliferation of an indicator cell population by antagonist custom derivative peptides is measured by the 3H-thymidine incorporation assay. Briefly, radiolabeled thymidine (1 microCi) is given to 20-50,000 cells undergoing proliferation in the presence of cytokines. The cell-incorporated radioactivity is measured by trapping cell-bound radioactivity to a glass-fiber filter using a conventional harvester machines (Example, Filtermate Universal Harvester from Perkin-Elmer), after which the radioactivity is measured using a b-counter (Example 1450, Trilux microplate scintillation counter).

### Example 4 - Method for Measuring Inhibition γc-Cytokine Activity by Assaying Incorporation of a Cell-Tracker Dye as a Marker of Cellular Proliferation

Indicator cells are incubated in the presence of a selected γc-cytokine or in the presence of a selected γc-cytokine and a selected custom derivative peptide. The cell population is then labeled in vitro using a cell-tracker dye, for example, CMFDA, C2925 from Invitrogen, and the decay of cellular green fluorescence at each cellular division is monitored using a flow-cytometer (for example, Beckton-Dickinson FACScalibur). Typically, in response to γc-cytokine stimulation 7~10 different peaks corresponding to the number of divisions that the cells have undergone will appear on the green fluorescence channel. Incubation of the cells with the selected γc-cytokine and antagonist custom derivative peptide reduces the number of peaks to only 1 to 3, depending on the degree of the inhibition.

### Example 5 - Inhibition of Intracellular Signaling by Custom Peptide Derivative Antagonists

In addition to stimulating cellular proliferation, binding of the γc-cytokines to their receptors causes a diverse array of intracellular events. (Rochman et al., 2009, Nat Rev Immunol 9:480-90; Pesu et al., 2005, Immunol Rev 203:127-42). Immediately after the cytokine binds to its receptor, a tyrosine kinase called Jak3 (Janus-kinase 3) is recruited to the receptor at the plasma membrane. This kinase phosphorylates the tyrosine residues of multiple proteins including the γc-subunit, STAT5 (Signal Transducer and Activator of Transcription 5) and subunits of the PI3 (Phosphatidylinositol 3) kinase. Among these, the phosphorylation of STAT5 has been implicated in many studies as being linked to the proliferation of cells initiated by the γc-cytokine (Hennighausen and Robinson, 2008, Genes Dev 22:711-21). In accordance with these published data, whether or not the BNZ-γ peptide inhibits the tyrosine phosphorylation of STAT5 molecule in PT-18β cells stimulated by IL-15 was examined (results shown in FIG. 4).

PT-18β cells were stimulated by IL-15 in the presence or absence of BNZ-γ peptide. Cytoplasmic proteins were extracted from the cells according to a conventional method (Tagaya et al., 1996, EMBO J 15:4928-39). The extracted cytoplasmic proteins were resolved using a standard SDS-PAGE (Sodium Dodecyl-Sulfate PolyAcrylamide Gel Electrophoresis) and the phosphorylation status was confirmed by an anti-phospho-STAT5 antibody (Cell Signaling Technology, Catalog # 9354, Danvers MA) using immunoblotting (See FIG. 4, top panel). To confirm that each lane represented a similar total protein load, the membrane was then stripped, and re-probed with an anti-STAT5 antibody (Cell Signaling Technology, Catalog # 9358) (See FIG. 4, bottom panel).

These results demonstrated that tyrosine phosphorylation of STAT5, a marker of signal transduction, was induced by IL-15 in PT-18β cells, and tyrosine phosphorylation of STAT5 was markedly reduced by the BNZ-γ peptide.

### Example 6 - Rational Design for γc-Antagonist Peptide Derivatives

Derivative peptides are prepared based from the core γc-box sequence (SEQ ID NO: 8) and the IL-2/IL-15 box sequence (SEQ ID NO: 9) shown in FIG. 1B by substituting the defined amino acids of the core sequence with amino acids having identical physico-chemical properties as designated in FIG. 2.

### Example 7 - Method of Identifying the Inhibitory Specificity of Antagonistic Custom Derivative Peptides

The IL-2, IL-9, and/or IL-15 γc-cytokine inhibitory specificity of antagonistic custom derivative peptides is determined by assaying the ability of a custom derivative peptide to inhibit the proliferative response of a cytokine-responsive cell line to each of the γc-cytokines. For example, a mouse cell line, CTLL-2, is used to determine if a candidate peptide inhibits the function of IL-2 and IL-15. PT-18(β) cells are used to determine if a candidate peptide inhibits the function of IL-4 and IL-9. PT-18 (7α) cells are used to determine if a candidate peptide inhibits the function of IL-7, and PT-18(21α) cells are used to determine if a candidate peptide inhibits the function of IL-21. PT-18(β) denotes a subclone of PT-18 cells that exogenously express human IL-2Rβ by gene transfection (Tagaya et al., 1996, EMBO J 15:4928-39), PT-18(7α) denotes a subclone that expresses human IL-7Rα by gene transfection and PT-18(21Rα) cells express human IL-21Rα.

Another alternative is to use other cell lines that respond to an array of cytokines. An example of this cell line in a human NK cell line NK92 that is commercially available by ATCC (catalog # CRL-2407). This cell line is an IL-2 dependent cell line that responds to other cytokines including IL-9, IL-7, IL-15, IL-12, IL-18, IL-21 (Gong et al., 1994, Leukemia 8:652-8; Kingemann et al., 1996, Biol Blood Marrow Transplant 2:68-75; Hodge DL et al., 2002, J Immunol 168:9090-8).

### Example 8 - Preparation of γc-Antagonist Peptides

Custom derivative γc-antagonist peptides are synthesized chemically by manual and automated processes.

Manual synthesis: Classical liquid-phase synthesis is employed, which involves coupling the carboxyl group or C-terminus of one amino acid to the amino group or N-terminus of another. Alternatively, solid-phase peptide synthesis (SPPS) is utilized.

Automated synthesis: Many commercial companies provide automated peptide synthesis for a cost. These companies use various commercial peptide synthesizers, including synthesizers provided by Applied Biosystems (ABI). Custom derivative γc-antagonist peptides are synthesized by automated peptide synthesizers.

### Example 9 - Biological Production of Custom Derivative γc-Antagonist Peptides Using Recombinant Technology

A custom derivative γc-antagonist peptide is synthesized biologically as a pro-peptide that consists of an appropriate tagging peptide, a signal peptide, or a peptide derived from a known human protein that enhances or stabilizes the structure of the BNZ-γ peptide and improves their biological activities. If desired, an appropriate enzyme-cleavage sequence proceeding to the N-terminus of the peptide shall be designed to remove the tag or any part of the peptide from the final protein.

A nucleotide sequence encoding the custom derivative peptide with a stop codon at the 3′ end is inserted into a commercial vector with a tag portion derived from thioredoxin of E. coli and a special peptide sequence that is recognized and digested by an appropriate proteolytic enzyme (for example, enterokinase) intervening between the tag portion and the nucleotide sequence encoding the custom derivative peptide and stop codon. One example of a suitable vector is the pThioHis plasmid available from Invitrogen, CA. Other expression vectors may be used.

### Example 10 - Conjugation of Custom Peptides and Derivative to Carrier Proteins for Immunization Purposes and Generation of Antibody Against the Custom Peptides

BNZ-γ or a derivative thereof are used to immunize animals to obtain polyclonal and monoclonal antibodies. Peptides are conjugated to the N- or the C-terminus of appropriate carrier proteins (for example, bovine serum albumin, Keyhole Limpet Hemocyanin (KLH), etc.) by conventional methods using Glutaraldehyde or m-Maleimidobenzoyl-N-Hydroxysuccinimide Ester. The conjugated peptides in conjunction with an appropriate adjuvant are then used to immunize animals such as rabbits, rodents, or donkeys. The resultant antibodies are examined for specificity using conventional methods. If the resultant antibodies react with the immunogenic peptide, they are then tested for the ability to inhibit individual γc-cytokine activity according to the cellular proliferation assays described in Examples 1-3. Due to the composite nature of the derivative peptides it is possible to generate a single antibody that recognizes two different cytokines simultaneously, because of the composite nature of these peptides.

### Example 11 - Method for Large Scale Production of Custom Derivative γc-Antagonist Peptides

Recombinant proteins are produced in large scale by the use of cell-free system as described (Takai et al., 2010, Curr Pharm Biotechnol 11:272-8). Briefly, cDNAs encoding the γc-antagonist peptide and a tag are subcloned into an appropriate vector (Takai et al., 2010, Curr Pharm Biotechnol 11:272-8), which is subjected to in vitro transcription, followed immediately by an in vitro translation to produce the tagged peptide. The pro-polypeptide is then purified using an immobilized antibody recognizing the tagged epitope, treated by the proteolytic enzyme and the eluate (which mostly contains the custom derivative peptide of interest) is tested for purity using conventional 18% Tricine-SDS-PAGE (Invitrogen) and conventional comassie staining. Should the desired purity of the peptide not be met (>98%), the mixture is subjected to conventional HPLC (high-performance liquid chromatography) for further purification.

### Example 12 - Use of Humanized NSG Mouse Model for the Therapeutic Investigation of Cytokine-Release Syndrome and Cytokine Storm Associated Disorders

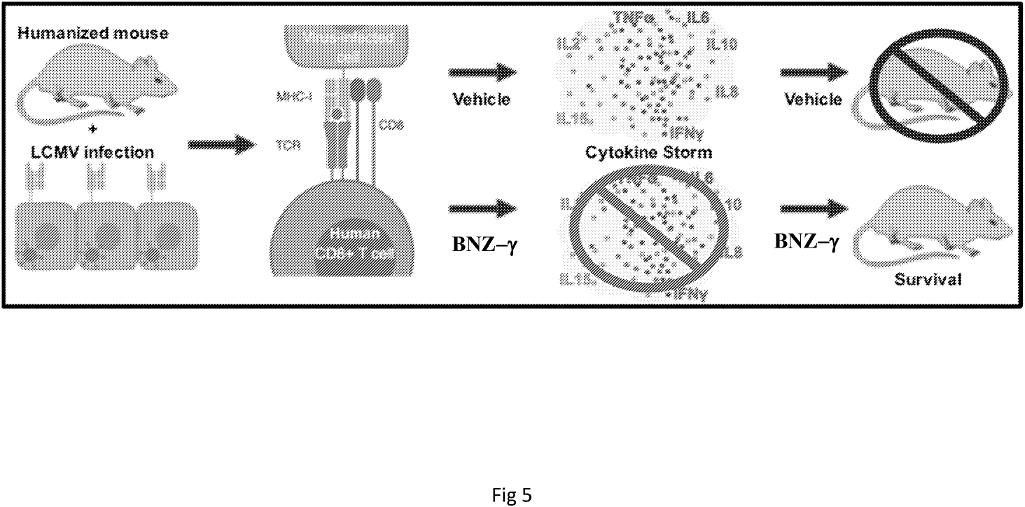

To study the BNZ-γ inhibition of cytokine storm the lymphocytic choriomeningitis virus (LCMV) was used. LCMV is a murine non-cytolytic virus with minimal immune response in wild type mice (Abdul-Hakeem, M.S. Viruses Teaching Immunology: Role of LCMV Model and Human Viral Infections in Immunological Discoveries. 2019 Viruses 11). Therefore, the degree of disease pathology depends on immune-mediated cytotoxicity. These challenges required the utilization of a humanized mouse model of the immune system to assess the BNZ-γ inhibitory effects of cytokine storm in the mouse. A major advancement for the in vivo study of human immunological systems was the development that a functional human immune system can be established in a severely immunodeficient mouse such as an immunocompromised NOD/Scid/Il2rg-/- (NSG) mouse. (Shultz et al., 2012, Nat Rev Immunol 12:786-98). NSG mice lack a functioning γc-subunit required for γc-cytokine signaling, are extremely deficient in lymphoid cells, and allow for very efficient human immune system engraftment after intraperitoneal administration of Ficoll-gradient purified human peripheral blood mononuclear cells (huPBMCs). Humanization prior to LCMV infection results in subsequent expansion of human lymphocytes and an unresolved immunopathology in the animal that provides an opportunity to study BNZ-γ inhibition of cytokine release syndrome (cytokine storm) in response to LCMV challenge (see FIG. 5).

### Example 13 - BNZ-γ Protects Against Cytokine Storm Induced Mortality

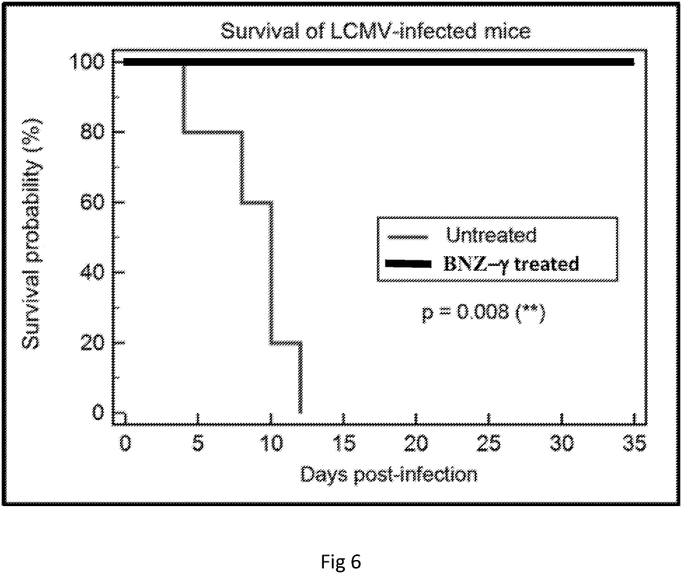

To test the effects of BNZ-γtreatment on cytokine storm, 11 NSG mice were each transplanted with 2 million huPBMCs and allowed 14 days to allow for human immune cell expansion. At day 15, mice were chronically infected with LCMV at 10⁶ pfu. On day 16, infected mice were administered PBS vehicle (n=5) or BNZ-γ at 2 mg/kg (n=6). The treatment was continued thereafter on a twice weekly dosing schedule, and animal mortality was monitored over a 5-week study period following initial LCMV infection. Complete BNZ-γ mediated protection of cytokine storm induced mortality at the drug dose of 2 mg/kg administered twice weekly throughout the duration of the study period was observed. All animals receiving PBS vehicle (n=5) died between days 4-12 of the study period. All animals receiving BNZ-γ treatment at 2 mg/kg (n=6) survived the 5-week study period (see FIG. 6).

### Example 14 - BNZ-γ Potently Blocks Induction of Pro-Inflammatory Cytokines of Cytokine Storm

It was hypothesized that BNZ-γ would protect cytokine storm induced mortality by blocking IL-2, IL-9, and/or IL-15 signaling simultaneously, which leads to the down-regulation of key pro-inflammatory cytokines that cause a fatal cytokine storm. To test this hypothesis, plasma levels of key pro-inflammatory cytokines downstream of the γc-cytokine signaling in each animal following LCMV challenge at early time-points within the first week following infection were assessed. Early time points were chosen to accurately track the levels of pro-inflammatory cytokines during the developmental phase of excess immune response to represent the cytokine storm pathogenic environment in our animal model and accurately represents the severe illness and mortality observed in cytokine storm associated disorders.

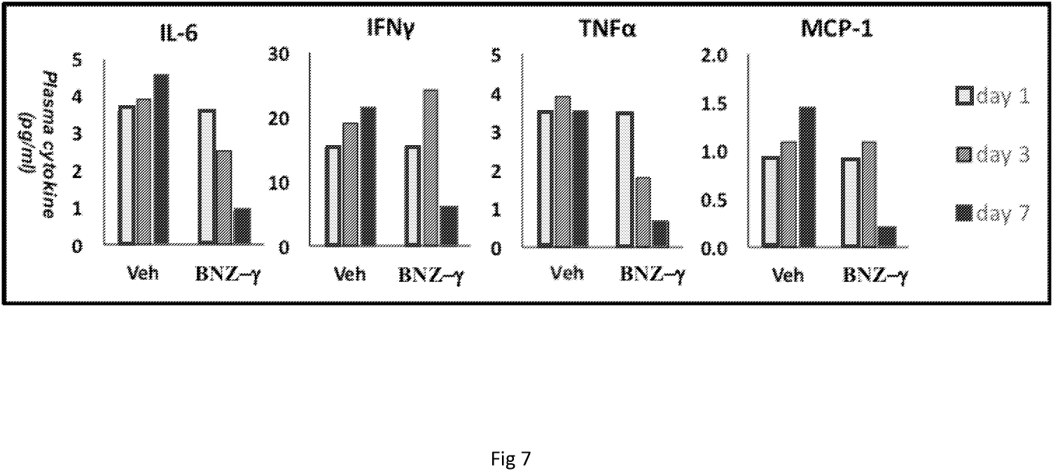

Blood collections were drawn from each PBS vehicle treated animal (n=5) and each BNZ-γ (2 mg/kg) treated animal (n=6) on days 1, 3, and 7 post-infection. The plasma concentrations of the pro-inflammatory cytokines IL-6, IFN-γ, TNF-α, and MCP-1 were assayed. A single mouse from the PBS vehicle control group died on day 4 post-infection, allowing for measurements from n=4 mice for day 7 post-infection in the untreated cohort. The average plasma level in pg/ml for each pro-inflammatory cytokine is reported at each time point for the control untreated group versus the BNZ-γ treated group (see FIG. 7). BNZ-γ exhibited potent inhibition of plasma levels of each pro-inflammatory measured by the end of the 7-day measurement period. For the PBS vehicle control treatment group, pro-inflammatory cytokines IL-6, IFN-γ, and MCP-1 showed consecutive incremental increases in plasma levels from days 1, 3, and 7 post-infection, whereas TNF-α displayed relatively constant levels ranging from ∼3.5-4 pg/ml over the duration of the measurement period after infection. BNZ-γ treatment displayed clear incremental decreases in both IL-6 and TNF-α plasma levels over the measurement period. All pro-inflammatory cytokines plasma levels in BNZ-γ treated animals were drastically reduced upon day 7 post-infection as compared to the PBS vehicle control group, with plasma levels dropping 6-fold for IL-6, 4.5-fold for IFN-γ, 7-fold for TNF-α, and 7.5-fold for MCP-1, respectively.

### Example 15 - Method of Treating Cytokine Release Syndrome (Cytokine Storm) in a Human Patient by Administration of a Therapeutic Compound

A human patient suffering from cytokine release syndrome (cytokine storm) is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 16 - Method of Treating Multiple Organ Dysfunction Syndrome in a Human Patient by Administration of a Therapeutic Compound

A human patient suffering from multiple organ dysfunction syndrome is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 17 - Method of Treating Systemic Inflammatory Response Syndrome in a Human Patient by Administration of a Therapeutic Compound

A human patient suffering from systemic inflammatory response syndrome is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 18 - Method of Treating Sepsis in a Human Patient by Administration of a Therapeutic Compound

A human patient suffering from sepsis (septic shock) is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 19 - Method of Treating Graft-Versus-Host Disease in a Human Patient by Administration of a Therapeutic Compound

A human patient suffering from graft-versus-host disease is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 20 - Method of Treating Cytokine Storm Associated Haploidentical Donor Transplantation in a Human Patient by Administration of a Therapeutic Compound

A human patient suffering from cytokine storm associated haploidentical donor transplantation is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 21 - Method of Treating Sarcoidosis in a Human Patient by Administration of a Therapeutic Compound

A human patient suffering from sarcoidosis is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 22 - Method of Treating Hemophagocytic Lymphohistiocytosis in a Human Patient by Administration of a Therapeutic Compound

A human patient suffering from hemophagocytic lymphohistiocytosis is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 23 - Method of Treating Vascular Leak Syndrome in a Human Patient by Administration of a Therapeutic Compound

A human patient suffering from vascular leak syndrome (systemic capillary leak syndrome) is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 24 - Method of Treating Stevens-Johnson Syndrome in a Human Patient by Administration of a Therapeutic Compound

A human patient suffering from Stevens-Johnson syndrome is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 25 - Method of Treating Toxic Epidermal Necrolysis in a Human Patient by Administration of a Therapeutic Compound

A human patient suffering from toxic epidermal necrolysis is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 26 - Method of Treating Cytokine Storm Associated Asthmatic Allergic Lung Inflammation in a Human Patient by Administration of a Therapeutic Compound

A human patient suffering from cytokine storm associated asthmatic allergic lung inflammation is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 27 - Method of Treating Cytokine Storm Associated Rhinosinusitis in a Human Patient by Administration of a Therapeutic Compound

A human patient suffering from cytokine storm associated rhinosinusitis is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 28 - Method of Treating Cytokine Storm Associated Viral Infection in a Human Patient by Administration of a Therapeutic Compound

A human patient suffering from cytokine storm associated viral infection (coronavirus infection, influenza infection, hantaviral infection, Epstein-Barr viral infection) is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 29 - Method of Treating Multisystem Inflammatory Syndrome in Children (MIS-C) Associated With COVID-19 in a Human Patient by Administration of a Therapeutic Compound

A human patient suffering from multisystem inflammatory syndrome in children (MIS-C) associated with COVID-19 is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 30 - Method of Treating Viral Hemorrhagic Fever in a Human Patient by Administration of a Therapeutic Compound

A human patient suffering from viral hemorrhagic fever (lassa hemorrhagic fever, Rift Valley fever, Crimean-Congo hemorrhagic fever, Yellow fever, Dengue fever, Ebola virus-induced fever, Marburg virus-induced fever) is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 31 - Method of Treating Cytokine Storm Associated HIV/HCV Coinfection Liver Fibrosis in a Human Patient by Administration of a Therapeutic Compound

A human patient suffering from cytokine storm associated HIV/HCV Coinfection liver fibrosis is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 32 - Method of Treating Cytokine Storm Associated Fungal Infection in a Human Patient by Administration of a Therapeutic Compound

A human patient suffering from cytokine storm associated fungal infection is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 33 - Method of Treating Pulmonary Aspergillosis in a Human Patient by Administration of a Therapeutic Compound

A human patient suffering from pulmonary aspergillosis is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 34 - Method of Treating Cytokine Storm Associated Bacterial Infection in a Human Patient by Administration of a Therapeutic Compound

A human patient suffering from cytokine storm associated bacterial infection (Staphylococcus infection, Streptoccus infection) is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 35 - Method of Treating Toxic Shock Syndrome in a Human Patient by Administration of a Therapeutic Compound

A human patient suffering from toxic shock syndrome is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 36 - Method of Treating Lyme Neuroborreliosis in a Human Patient by Administration of a Therapeutic Compound

A human patient suffering from lyme neuroborreliosis is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 37 - Method of Treating Juvenile Idiopathic Arthritis in a Human Patient by Administration of a Therapeutic Compound

A human patient suffering from juvenile idiopathic arthritis (Still’s disease) is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 38 - Method of Treating Macrophage Activation Syndrome in a Human Patient by Administration of a Therapeutic Compound

A human patient suffering from macrophage activation syndrome is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 39 - Method of Treating Sjögren’s Syndrome in a Human Patient by Administration of a Therapeutic Compound

A human patient suffering from Sjögren’s syndrome is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 40 - Method of Treating Systemic Sclerosis in a Human Patient by Administration of a Therapeutic Compound

A human patient suffering from systemic sclerosis is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 41 - Method of Treating Inflammatory Myopathies in a Human Patient by Administration of a Therapeutic Compound

A human patient suffering from inflammatory myopathies is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 42 - Method of Treating Systemic Vasculitides in a Human Patient by Administration of a Therapeutic Compound

A human patient suffering from systemic vasculitides is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 43 - Method of Treating Giant Cell Arteritis in a Human Patient by Administration of a Therapeutic Compound

A human patient suffering from giant cell arteritis (Horton disease, cranial arteritis, temporal arteritis) is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 44 - Method of Treating T-Cell Based Immunotherapy-Induced Cytokine Storm in a Human Patient by Administration of a Therapeutic Compound

A human patient suffering from T-cell based immunotherapy (chimeric antigen receptor T-cell therapy, T-cell bispecific antibody therapy) - induced cytokine storm is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 45 - Method of Treating Immune Effector Cell-Associated Neurotoxicity Syndrome in a Human Patient by Administration of a Therapeutic Compound

A human patient suffering from immune effector cell-associated neurotoxicity syndrome is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 46 - Method of Treating Pulmonary Infiltrate in a Human Patient by Administration of a Therapeutic Compound

A human patient suffering from pulmonary infiltrate is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 47 - Method of Treating Adult Respiratory Distress Syndrome in a Human Patient by Administration of a Therapeutic Compound

A human patient suffering from adult respiratory distress syndrome is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 48 - Method of Treating Interstitial Lung Disease in a Human Patient by Administration of a Therapeutic Compound

A human patient suffering from interstitial lung disease is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### Example 49 - Method of Treating Pneumonia in a Human Patient by Administration of a Therapeutic Compound

A human patient suffering from pneumonia (bacterial pneumonia, fungal pneumonia, parasitic-induced pneumonia, viral pneumonia, community-acquired pneumonia, acute interstitial pneumonia) is identified. An effective dose, as determined by the physician, of a therapeutic compound, for example, a composite peptide comprising the sequence of BNZ-γ, or a derivative thereof, or a combination of said therapeutic compounds is administered to the patient for a period of time determined by the physician. Treatment is determined to be effective if patient’s symptoms improve or if the progression of the disease has been stopped or slowed down. It is determined that the patient is treated.

### REFERENCES

All references disclosed herein as well as listed below are incorporated by reference in their entireties.

Abboud, R. et al. Severe Cytokine-Release Syndrome after T Cell-Replete Peripheral Blood Haploidentical Donor Transplantation Is Associated with Poor Survival and Anti-IL-6 Therapy Is Safe and Well Tolerated. 2016 Biol Blood Marrow Transplant 22:1851-60.

Abdul-Hakeem, M.S. Viruses Teaching Immunology: Role of LCMV Model and Human Viral Infections in Immunological Discoveries. 2019 Viruses 11.

Abe, R. Immunological Response in Stevens-Johnson Syndrome and Toxic Epidermal Necrolysis. 2015 J Dermatol 42:42-8.

Adusumilli, P.S. et al. Regional Delivery of Mesothelin-Targeted CAR T Cell Therapy Generates Potent and Long-Lasting CD4-Dependent Tumor Immunity. 2014 Sci Transl Med 6:261ra151.

Agostini, C. et al. Role of IL-15, IL-2, and Their Receptors in the Development of T Cell Alveolitis in Pulmonary Sarcoidosis. 1996 J Immunol 157:910-8.

Agostini, C. et al. New Pathogenetic Insights into the Sarcoid Granuloma. 2000 Curr Opin Rheumatol 12:71-6.

Agouridakis, P. et al. Association between Increased Levels of IL-2 and IL-15 and Outcome in Patients with Early Acute Respiratory Distress Syndrome. 2002 Eur J Clin Invest 32:862-7.

Allison, R.D. et al. Association of Interleukin-15-Induced Peripheral Immune Activation with Hepatic Stellate Cell Activation in Persons Coinfected with Hepatitis C Virus and HIV. 2009 J Infect Dis 200:619-23.

Antin, J.H. et al. Cytokine Dysregulation and Acute Graft-versus-Host Disease. 1992 Blood 80:2964-8.

Antony, P.A., Paulos, C.M., Ahmadzadeh, M., Akpinarli, A., Palmer, D.C., Sato, N., Kaiser A., Heinrichs, C.S., Klebanoff, C.A., Tagaya, Y., and Restifo, NP., Interleukin-2-dependent mechanisms of tolerance and immunity in vivo. 2006 J Immunol 176:5255-66.

Arras, M. et al. Interleukin-9 Reduces Lung Fibrosis and Type 2 Immune Polarization Induced by Silica Particles in a Murine Model. 2001 Am J Respir Cell Mol Biol 24:368-75.

Assier, E. et al. NK Cells and Polymorphonuclear Neutrophils Are Both Critical for IL-2-Induced Pulmonary Vascular Leak Syndrome. 2004 J Immunol 172:7661-8.

Awwad, S. and Angkawinitwong, U., Overview of Antibody Drug Delivery. 2018 Pharmaceutics 10:83.

Azimi, N., Nagai, M., Jacobson, S., Waldmann, T.A., IL-15 plays a major role in the persistence of Tax-specific CD8 cells in HAM/TSP patients. 2001 Proc Natl Acad Sci 98:14559-64.

Azimi, N., Mariner J., Jacobson S., Waldmann T.A., How does interleukin 15 contribute to the pathogenesis of HTLV type-1 associated myelopathy/tropical spastic paraparesis? 2000 AIDS Res Hum Retroviruses 16:1717-22.

Azimi, N., Jacobson, S., Leist, T., Waldmann, T.A., Involvement of IL-15 in the pathogenesis of human T lymphotropic virus type-I-associated myelopathy/tropical spastic paraparesis: implications for therapy with a monoclonal antibody directed to the IL-2/15R beta receptor. 1999 J Immunol 163:4064-72.

Azimi, N., Brown, K., Bamford, R.N., Tagaya, Y., Siebenlist, U., Waldmann, T.A., Human T cell lymphotropic virus type I Tax protein trans-activates interleukin 15 gene transcription through an NF-kappaB site. 1998 Proc Natl Acad Sci 95:2452-7.

Bae, H. et al. Immune Response during Adverse Events after 17D-Derived Yellow Fever Vaccination in Europe. 2008 J Infect Dis 197:1577-84.

Baird, G.S. et al. Multiplex immunoassay analysis of cytokines in idiopathic inflammatory myopathy. 2008 Arch Pathol Lab Med 132:232-8.

Baize, S. et al. Early and Strong Immune Responses Are Associated with Control of Viral Replication and Recovery in Lassa Virus-Infected Cynomolgus Monkeys. 2009 J Virol 83:5890-903.

Banadyga, L. et al. The Cytokine Response Profile of Ebola Virus Disease in a Large Cohort of Rhesus Macaques Treated With Monoclonal Antibodies. 2019 Open Forum Infect Dis 6:ofz046.

Baraut, J. et al. Relationship between cytokine profiles and clinical outcomes in patients with systemic sclerosis. 2010 Autoimmun Rev 10:65-73.

Barnes, P.J. et al. The Cytokine Network in Asthma and Chronic Obstructive Pulmonary Disease. 2008 J Clin Invest 118:3546-56.

Bazan, J.F., Hematopoietic receptors and helical cytokines. 1990 Immunol Today 11:350-354.

Becker, T.C. et al. Interleukin 15 Is Required for Proliferative Renewal of Virus-Specific Memory CD8 T Cells. 2002 J Exp Med 195:1541-8.

Belani, R. et al. T Cell Activation and Cytokine Production in Anti-CD3 Bispecific Antibody Therapy. 1995 J Hematother 4:395-402.

Belhadjer, Z. et al. Acute heart failure in multisystem inflammatory syndrome in children (MIS-C) in the context of global SARS-CoV-2 pandemic. 2020 Circulation in press.

Bequignon, E. et al. Pathogenesis of chronic rhinosinusitis with nasal polyps: role of IL-6 in airway epithelial cell dysfunction. 2020 J Transl Med 18:136.

Bettini, M., and Vignali, D.A., Regulatory T cells and inhibitory cytokines in autoimmunity. 2009 Curr Opin Immunol 21:612-8.

Bixler, S. et al. The Role of Cytokines and Chemokines in Filovirus Infection. 2015 Viruses 7:5489-507.

Biber, J.L. et al. Administration of two macrophage-derived interferon-gamma-inducing factors (IL-12 and IL-15) induces a lethal systemic inflammatory response in mice that is dependent on natural killer cells but does not require interferon-gamma. 2002 216:31-42.

Björkström, N.K. et al. Rapid Expansion and Long-Term Persistence of Elevated NK Cell Numbers in Humans Infected with Hantavirus. 2010 J Exp Med 208:13-21.

Blackwell, T.S. et al. Sepsis and Cytokines: Current Status. 1996 Br J Anaesth 77:110-7.

Blaser, B.W., Roychowdhury, S, Kim, D.J., Schwind, N.R., Bhatt, D., Yuan, W., Kusewitt, D.F., Ferketich, A.K., Caligiuri, M.A., Guimond, M. Donor-derived IL-15 is critical for acute allogeneic graft-versus-host disease. 2005 Blood 105:894-901.

Blaser, B.W. et al. Trans-Presentation of Donor-Derived Interleukin 15 Is Necessary for the Rapid Onset of Acute Graft-versus-Host Disease but Not for Graft-versus-Tumor Activity. 2006 Blood 108:2463-9.

Bodd, M., Raki, M., Tollefsen, S., Fallang, L.E., Bergseng, E., Lundin, K.E., Sollid, L.M., HLA-DQ2-restricted gluten-reactive T cells produce IL-21 but not IL-17 or IL-22. 2010 Mucosal Immunol 3:594-601.

Bonifant, C.L. et al. Toxicity and Management in CAR T-Cell Therapy. 2016 Mol Ther Oncolytics 3:16011.

Boumba, D. et al. Cytokine mRNA expression in the labial salivary gland tissues from patients with primary Sjögrens Syndrome. 1995 Br J Rheumatol 34:326-33.

Braun, M. et al. NK Cell Activation in Human Hantavirus Infection Explained by Virus-Induced IL-15/IL15Rα Expression. 2014 PLoS Pathog 10:e1004521.

Brisse, E. et al. Hemophagocytic Lymphohistiocytosis (HLH): A Heterogeneous Spectrum of Cytokine-Driven Immune Disorders. 2015 Cytokine Growth Factor Rev 26:263-80.

Brudno, J.N. et al. Toxicities of Chimeric Antigen Receptor T Cells: Recognition and Management. 2016 Blood 127:3321-30.

Bruminhent, J. et al. Acute Interstitial Pneumonia (Hamman-Rich Syndrome) as a Cause of Idiopathic Acute Respiratory Distress Syndrome. 2011 Case Rep Med 2011:628743.

Buchweitz, J.P. et al. Time-Dependent Airway Epithelial and Inflammatory Cell Responses Induced by Influenza Virus A/PR/8/34 in C57BL/6 Mice. 2007 Toxicol Pathol 35:424-35.

Cahill, L.A. et al. Circulating Factors in Trauma Plasma Activate Specific Human Immune Cell Subsets. 2020 Injury 51:819-29.

Caproni, M. et al. Expression of Cytokines and Chemokine Receptors in the Cutaneous Lesions of Erythema Multiforme and Stevens-Johnson Syndrome/Toxic Epidermal Necrolysis. 2006 Br J Dermatol 155:722-8.

Carding, S.R. et al. Activation of Cytokine Genes in T Cells during Primary and Secondary Murine Influenza Pneumonia. 1993 J Exp Med 177:475-82.

Carey, P.D. et al. Neutrophil Activation, Vascular Leak Toxicity, and Cytolysis during Interleukin-2 Infusion in Human Cancer. 1997 Surgery 122:918-26.

Cerar, T. et al. Diagnostic value of cytokines and chemokines in lyme neuroborreliosis. 2013 Clin Vaccine Immunol 20:1578-84.

Channappanavar, R. et al. Pathogenic Human Coronavirus Infections: Causes and Consequences of Cytokine Storm and Immunopathology. 2017 Semin Immunopathol 39:529-39.

Chaturvedi, U.C. et al. Cytokine Cascade in Dengue Hemorrhagic Fever: Implications for Pathogenesis. 2000 FEMS Immunol Med Microbiol 28:183-8.

Chen, J. et al. Cellular Immune Responses to Severe Acute Respiratory Syndrome Coronavirus (SARS-CoV) Infection in Senescent BALB/c Mice: CD4 T Cells Are Important in Control of SARS-CoV Infection. 2009 J Virol 84:1289-1301.

Chen, X. et al. Elevated Cytokine Levels in Tears and Saliva of Patients with Primary Sjögren’s Syndrome Correlate with Clinical Ocular and Oral Manifestations. 2019 Sci Rep 9:7319.

Chi, Y. et al. Cytokine and Chemokine Levels in Patients Infected With the Novel Avian Influenza A (H7N9) Virus in China. 2013 J Infect Dis 208:1962-7.

Chien, J. et al. Temporal Changes in Cytokine/Chemokine Profiles and Pulmonary Involvement in Severe Acute Respiratory Syndrome. 2006 Respirology 11:715-22.

Chik, K.W., Li, K., Pong, H., Shing, M.M., Li, C.K., Yuen, P.M. Elevated serum interleukin-15 level in acute graft-versus-host disease after hematopoietic cell transplantation. 2003 J Pediatr Hematol Oncol 25:960-4.

Chiotos, K. et al. Multisystem Inflammatory Syndrome in Children during the COVID-19 pandemic: a case series. 2020 J Pediatr Infect Dis Soc in press.

Chung, W.H. et al. Recent Advances in the Genetics and Immunology of Stevens-Johnson Syndrome and Toxic Epidermal Necrosis. 2012 J Dermatol Sci 66:190-6.

Cicardi, M. et al. The Systemic Capillary Leak Syndrome: Appearance of Interleukin-2-Receptor-Positive Cells during Attacks. 1990 Ann Intern Med 113:475-7.

Ciccia, F. et al. Difference in the expression of IL-9 and IL-17 correlates with different histological pattern of vascular wall injury in giant cell arteritis. 2015 Rheumatology 54:1596-604.

Clay, C.C. et al. Severe Acute Respiratory Syndrome-Coronavirus Infection in Aged Nonhuman Primates Is Associated with Modulated Pulmonary and Systemic Immune Responses. 2014 Immun Ageing 11:4; 1742-4933-11-4.

Cron, R.Q. et al. Cytokine Storm Syndrome. 2019 Cham: Springer International Publishing.

De Paepe, B. et al. Scanning for Therapeutic Targets within the Cytokine Network of Idiopathic Inflammatory Myopathies. 2015 Int J Mol Sci 16:18683-713.

DΈlia, R.V., Harrison, K., Oyston, P.C., Lukaszewski, R.A., Clark, G.C. Targeting the “cytokine storm” for therapeutic benefit. 2013 Clin Vaccine Immunol 20:319-27.

DeMaria, A. et al. CD3+4-8-WT31-(T Cell Receptor gamma+) Cells and Other Unusual Phenotypes Are Frequently Detected among Spontaneously Interleukin 2-Responsive T Lymphocytes Present in the Joint Fluid in Juvenile Rheumatoid Arthritis. A Clonal Analysis. 1987 Eur J Immunol 17:1815-9.

DeRezende, L.C., Silva I.V., Rangel, L.B., Guimaraes, M.C., Regulatory T cells as a target for cancer therapy. 2010 Arch Immunol Ther Exp 58:179-90.

Dolinger, M.T. et al. Pediatric Crohn’s Disease and Multisystem Inflammatory Syndrome in Children (MIS-C) and COVID-19 Treated with Infliximab. 2020 J Pediatr Gastroenterol Nutr in press.

Dong, Q. et al. IL-9 Induces Chemokine Expression in Lung Epithelial Cells and Baseline Airway Eosinophilia in Transgenic Mice. 1999 Eur J Immunol 29:2130-9.

Duan, C. et al. Regulatory mechanisms, prophylaxis and treatment of vascular leakage following severe trauma and shock. 2017 Mil Med Res 4:11.

Dubois, S., Mariner, J., Waldmann, T.A., Tagaya, Y., IL-15Ralpha recycles and presents IL-15 In trans to neighboring cells. 2002 Immunity 17:537-47.

Endo, S. et al. Two types of septic shock classified by the plasma levels of cytokines and endotoxin. 1992 Circ Shock 38:264-74.

Engelmann, F. et al. Pathophysiologic and Transcriptomic Analyses of Viscerotropic Yellow Fever in a Rhesus Macaque Model. 2014 PLoS Negl Trop Dis 8:e000329

Ermler, M.E. et al. RNA Helicase Signaling Is Critical for Type I Interferon Production and Protection against Rift Valley Fever Virus during Mucosal Challenge. 2013 J Virol 87:4846-60.

Fadeel, B. et al. Induction of Apoptosis and Caspase Activation in Cells Obtained from Familial Haemophagocytic Lymphohistiocytosis Patients. 1999 Br J Haematol 106:406-15.

Falasca, L. et al. Molecular Mechanisms of Ebola Virus Pathogenesis: Focus on Cell Death. 2015 Cell Death Differ 22:1250-9.

Fang J., Hu, C., Hong, M., Wu, Q., You, Y., Zhong, Z., Li, W., Zou, P., Hu, Y., Prophylactic effects of interleukin-2 receptor antagonists against graft-versus-host disease following unrelated donor peripheral blood stem cell transplantation. 2012 Biol Blood Marrow Transplant 18:754-62.

Faulkner, L. et al. The Mechanism of Superantigen-Mediated Toxic Shock: Not a Simple Th1 Cytokine Storm. 2005 J Immunol 175:6870-7.

Fehniger, T.A., Suzuki, K., Ponnappan, A., VanDeusen, J.B., Cooper, M.A., Florea, S.M., Freud, A.G., Robinson, M.L., Durbin, J., Caligiuri, M.A., Fatal leukemia in interleukin 15 transgenic mice follows early expansions in natural killer and memory phenotype CD8+ T cells. 2001 J Exp Med 193:219-31.

Forrester, J.M. et al. TCR Expression of Activated T Cell Clones in the Lungs of Patients with Pulmonary Sarcoidosis. 1994 J Immunol 153:4291-302.

Fox, R.I. et al. Cytokine MRNA Expression in Salivary Gland Biopsies of Sjögren’s Syndrome. 1994 J Immunol 152:5532-9.

Friberg, H. et al. Protective versus Pathologic Pre-Exposure Cytokine Profiles in Dengue Virus Infection. 2018 PLoS Negl Trop Dis 12:e0006975.

Funke, I. et al. Capillary Leak Syndrome Associated with Elevated IL-2 Serum Levels after Allogeneic Bone Marrow Transplantation. 1994 Ann Hematol 68:49-52.

Gogishvili, T. et al. Rapid regulatory T-cell response prevents cytokine storm in CD28 superagonist treated mice. 2009 PLoS One 4:e4643.

Gong, J.H. et al. Characterization of a human cell line (NK-92) with phenotypical and functional characteristics of activated natural killer cells. 1994 Leukemia 8:652-8.

Gono, T. et al. Cytokine profiles in polymyositis and dermatomyositis complicated by rapidly progressive or chronic interstitial lung disease. 2014 Rheumatology 53:2196-203.

Gourh, P. et al. Polymorphisms in TBX21 and STAT4 increase the risk of systemic sclerosis: evidence of possible gene-gene interaction and alterations in Th1/Th2 cytokines. 2009 Arthritis Rheum 60:3794-806.

Gruss, H.J. et al. Human fibroblasts express functional IL-2 receptors formed by the IL-2R alpha- and beta-chain subunits: association of IL-2 binding with secretion of the monocyte chemoattractant protein-1. 1996 J Immunol 157:851-7.

Guggino, G. et al. Interleukin-9 over-expression and T helper 9 polarization in systemic sclerosis patients. 2017 Clin Exp Immunol 190:208-16.

Guo, J. et al. The Serum Profile of Hypercytokinemia Factors Identified in H7N9-Infected Patients Can Predict Fatal Outcomes. 2015 Sci Rep 5:srep10942.

Guo, J. et al. Coronavirus Disease 2019 (COVID-19) and Cardiovascular Disease: A Viewpoint on the Potential Influence of Angiotensin-Converting Enzyme Inhibitors/Angiotensin Receptor Blockers on Onset and Severity of Severe Acute Respiratory Syndrome Coronavirus 2 Infection. 2020 J Am Heart Assoc 9:e0162219.

Guo, Y. et al. IL-15 Superagonist-Mediated Immunotoxicity: Role of NK Cells and IFN-γ. 2015 J Immunol 195:2353-64.

Guo, Y. et al. IL-15 Enables Septic Shock by Maintaining NK Cell Integrity and Function. 2017 J Immunol 198:1320-33.

Guo, Y. et al. The Origin, Transmission and Clinical Therapies on Coronavirus Disease 2019 (COVID-19) Outbreak - an Update on the Status. 2020 Military Med Res 7:1.

Han, S.H. et al. The acute respiratory distress syndrome: from mechanism to translation. 2015 J Immunol 194:855-60.

Han, X.C. et al. Cytokine profiles as novel diagnostic markers of Epstein-Barr virus-associated hemophagocytic lymphohistiocytosis in children. 2017 J Crit Care 39:72-7.

Hao, W. et al. Mathematical Model of Sarcoidosis. 2014 Proc Natl Acad Sci 111:16065-70.

Harris, D.P. et al. Reciprocal Regulation of Polarized Cytokine Production by Effector B and T Cells. 2000 Nat Immunol 1:475-82.

Haugen, J. et al. Cytokine Concentrations in Plasma from Children with Severe and Non-Severe Community Acquired Pneumonia. 2015 PLoS One 10:e0138978.

Hechinger, A.K. et al. Therapeutic Activity of Multiple Common γ-Chain Cytokine Inhibition in Acute and Chronic GVHD. 2015 Blood 125:570-80.

Hennighausen, L., Robinson, G.W., Interpretation of cytokine signaling through the transcription factors STAT5A and STAT5B. 2008 Genes Dev 22:711-21.

Hodge, D.L. et al. IL-2 and IL-12 alter NK cell responsiveness to IFN-gamma-inducible protein 10 by down-regulating CXCR3 expression. 2002 J Immunol 168:6090-8.

Hogaboam, C.M. et al. Differential monocyte chemoattractant protein-1 and chemokine receptor 2 expression by murine lung fibroblasts derived from Th1- and Th2-type pulmonary granuloma models. 1999 J Immunol 163:2193-201.

Hondowicz, B.D. et al. Interleukin-2-Dependent Allergen-Specific Tissue-Resident Memory Cells Drive Asthma. 2016 Immunity 44:155-66.

Hornef, M.W. et al. Cytokine production in a whole-blood assay after Epstein-Barr virus infection in vivo. 1995 Clin Diagn Lab Immunol 2:209-13.

Huang, C. et al. Clinical Features of Patients Infected with 2019 Novel Coronavirus in Wuhan, China. 2020 Lancet 395:497-506.

Huang, Y. et al. Innate and Adaptive Immune Responses in Patients with Pandemic Influenza A(H1N1)pdm09. 2013 Arch Virol 158:2267-72.

Hunninghake, G.W. et al. Mechanisms of Hypergammaglobulinemia in Pulmonary Sarcoidosis. Site of Increased Antibody Production and Role of T Lymphocytes. 1981 J Clin Invest 67:86-92.

Jabri, B. et al. IL-15 Functions as a Danger Signal to Regulate Tissue-Resident T Cells and Tissue Destruction. 2015 Nat Rev Immunol 15:771-83.

Jarvis, J.N. et al. Neutrophils: the Forgotten Cell in JIA Disease Pathogenesis. 2007 Pediatr Rheumatol Online 5:13.

Jia, L. et al. Detection of IL-9 Producing T Cells in the PBMCs of Allergic Asthmatic Patients. 2017 BMC Immunol 18:38.

Jillella, A.P. et al. Non-Hodgkins Lymphoma Presenting as Anasarca: Probably Mediated by Tumor Necrosis Factor Alpha (TNF-α). 2000 Leuk Lymphoma 38:419-22.

Jiménez-Sousa, M.A. et al. IL15polymorphism Is Associated with Advanced Fibrosis, Inflammation-Related Biomarkers and Virological Response in Human Immunodeficiency Virus/Hepatitis C Virus Coinfection. 2016 Liver Int 36:1258-66.

Kahaleh, M.B. et al. Interleukin-2 in scleroderma: correlation of serum level with extent of skin involvement and disease duration. 1989 Ann Intern Med 110:446-50.

Kalyan, S. et al. Human Peripheral Gammadelta T Cells Potentiate the Early Proinflammatory Cytokine Response to Staphylococcal Toxic Shock Syndrome toxin-1. 2004 J Infect Dis 189:1892-6.

Kappler, J. et al. V Beta-Specific Stimulation of Human T Cells by Staphylococcal Toxins. 1989 Science 244:811-3.

Khan, A.A. et al. IL-2 Regulates SEB Induced Toxic Shock Syndrome in BALB/c Mice. 2009 PLoS One 4:e8473.

Kim, Y.S. et al. Targeting the IL-15 Receptor with an Antagonist IL-15 Mutant/Fc gamma2a Protein Blocks Delayed-Type Hypersensitivity. 1998 J Immunol 160:5742-8.

Kimber, I. et al. Toxic Shock Syndrome: Characterization of Human Immune Responses to TSST-1 and Evidence for Sensitivity Thresholds. 2013 Tox Sci 134:49-63.

Kimura, A. et al. The Postoperative Serum Interleukin-15 Concentration Correlates with Organ Dysfunction and the Prognosis of Septic Patients Following Emergency Gastrointestinal Surgery. 2012 J Surg Res 175:e83-8.

Klingemann, H.G. et al. A cytotoxic NK-cell line (NK-92) for ex vivo purging of leukemia from blood. 1996 Biol Blood Marrow Transplant 2:68-75.

Klingström, J. et al. Innate and Adaptive Immune Responses against Human Puumala Virus Infection: Immunopathogenesis and Suggestions for Novel Treatment Strategies for Severe Hantavirus-Associated Syndromes. 2019 J Intern Med 285:510-23.

Koh, Y.Y. et al. Levels of Interleukin-2, Interferon-gamma, and Interleukin-4 in Bronchoalveolar Lavage Fluid From Patients With Mycoplasma Pneumonia: Implication of Tendency Toward Increased Immunoglobulin E Production. 2001 Pediatrics 107:E39.

Kooy-Winkelaar, Y.M, Bouwer, D., Janssen, G.M., Thompson, A., Brugman, M.H., Schmitz, F., de Ru, A.H., van Gils, T., Bouma, G., van Rood, J.J. et al. CD4 T-cell cytokines synergize to induce proliferation of malignant and nonmalignant intraepithelial lymphocytes. 2017 Proc Natl Acad Sci 114:E980-9.

Krakauer, T. et al. Immune Response to Staphylococcal Superantigens. 1999 Immunol Res 20:163-73.

Krause, C.D. and Pestka, S., Evolution of the Class 2 cytokines and receptors, and discovery of new friends and relatives. 2005 Pharmacol Ther 106:299-346.

Krieg, C. et al. Improved IL-2 immunotherapy by selective stimulation of IL-2 receptors on lymphocytes and endothelial cells. 2010 Proc Natl Acad Sci 107:11906-11.

Kundig, T.M., Schorle, H., Bachmann, M.F., Hengartener, H., Zinkernagel, R.M., Horak, I., Immune Responses of the interleukin-2-deficient mice. 1993 Science 262:1059-61.

Kurane, I. et al. Activation of T Lymphocytes in Dengue Virus Infections. High Levels of Soluble Interleukin 2 Receptor, Soluble CD4, Soluble CD8, Interleukin 2, and Interferon-Gamma in Sera of Children with Dengue. 1991 J Clin Invest 88:1473-80.

Kushner, L.E. et al. Immune Biomarker Differences and Changes Comparing HCV Mono-Infected, HIV/HCV Co-Infected, and HCV Spontaneously Cleared Patients. 2013 PLoS One 8:e60387.

Lamparello, A.J. et al. Severely Injured Trauma Patients with High Circulating IL-15 Levels Display Worse Outcomes and Distinct Inflammatory Profiles, Suggesting a Role for Natural Killer Cell Activation. 2019 J Am Coll Surg 229:S310.

Lashine, Y.A. et al. Correcting the Expression of MiRNA-155 Represses PP2Ac and Enhances the Release of IL-2 in PBMCs of Juvenile SLE Patients. 2015 Lupus 24:240-7.

Leahy, T.R. et al. Interleukin-15 Is Associated with Disease Severity in Viral Bronchiolitis. 2015 Eur Resp J 47:212-22.

Lee, Y.G. et al. Regulation of CAR T Cell-Mediated Cytokine Release Syndrome-like Toxicity Using Low Molecular Weight Adapters. 2019 Nat Commun 10:2681.

Lentsch, A.B. et al. Mechanisms of Leukocyte-Mediated Tissue Injury Induced by Interleukin-2. 1999 Cancer Immunol Immunother 47:243-8.

Lerkvaleekul, B. et al. Macrophage Activation Syndrome: Early Diagnosis Is Key. 2018 Open Access Rheumatol 10:117-28.

Lesur, O. et al. Interleukin-2 Involvement in Early Acute Respiratory Distress Syndrome: Relationship with Polymorphonuclear Neutrophil Apoptosis and Patient Survival. 2000 Crit Care Med 12:3814-22.

Li, H. et al. Structure-Function Studies of T-Cell Receptor-Superantigen Interactions. 1998 Immunol Rev 163:177-86.

Li, J. et al. CD3 bispecific antibody-induced cytokine release is dispensable for cytotoxic T cell activity. 2019 Sci Transl Med 11:eaax8861.

Li, T. et al. IL-9 Deficiency Promotes Pulmonary Th17 Response in Murine Model of Pneumocystis Infection. 2018 Front Immunol 9:1118.

Li, Y. et al. Coronavirus Neurovirulence Correlates with the Ability of the Virus To Induce Proinflammatory Cytokine Signals from Astrocytes and Microglia. 2004 J Virol 78:3398-406.

Lin, H. et al. Expression and regulation of interleukin-9 in chronic rhinosinusitis. 2015 Am J Rhinol Allergy 29:e18-23.

Lin, K.L. et al. Temporal Characterization of Marburg Virus Angola Infection Following Aerosol Challenge in Rhesus Macaques. 2015 J Virol 89:9875-85.

Linde, A. et al. Serum levels of lymphokines and soluble cellular receptors in primary Epstein-Barr virus infection and in patients with chronic fatigue syndrome. 1992 J Infect Dis 165:994-1000.

Link B.K. et al. Anti-CD3-based Bispecific Antibody Designed for Therapy of Human B-cell Malignancy can Induce T-cell Activation by Antigen-Dependent and Antigen-Independent Mechanisms. 1998 Int J Cancer 77:251-6.

Lisi, S. et al. Sjögrens Syndrome Autoantibodies Provoke Changes in Gene Expression Profiles of Inflammatory Cytokines Triggering a Pathway Involving TACE/NF-KB. 2012 Lab Invest 92:615-24.

Liu, J. et al. Overlapping and Discrete Aspects of the Pathology and Pathogenesis of the Emerging Human Pathogenic Coronaviruses SARS-CoV, MERS-CoV, and 2019-NCoV. 2020 J Med Virol 92:491-4.

Liu, J. et al. Longitudinal Characteristics of Lymphocyte Responses and Cytokine Profiles in the Peripheral Blood of SARS-CoV-2 Infected Patients. 2020 EBioMedicine 55:102763.

Liu, M. et al. Differences in Inflammatory Marker Patterns for Adult Community-Acquired Pneumonia Patients Induced by Different Pathogens. 2018 Clin Respir J 12:974-85.

Logan, T.F. et al. Increased disease activity in a patient with sarcoidosis after high dose interleukin 2 treatment for metastatic renal cancer. 2005 Thorax 60:610-1.

Lotz, M. et al. Release of lymphokines after Epstein Barr virus infection in vitro. I. Sources of and kinetics of production of interferons and interleukins in normal humans. 1986 J Immunol 136:3636-42.

Lourdes, L.S. et al. Systemic Capillary Leak Syndrome as an Initial Presentation of ALK-Negative Anaplastic Large Cell Lymphoma. 2012 Case Rep Hematol 2012:954201.

Macaubas, C. et al. Oligoarticular and Polyarticular JIA: Epidemiology and Pathogenesis. 2009 Nat Rev Rheumatol 5:616-26.

Mahallawi, W.H. et al. MERS-CoV Infection in Humans Is Associated with a pro-Inflammatory Th1 and Th17 Cytokine Profile. 2018 Cytokine 104:8-13.

Makarevich, A. et al. Interleukin-2 (IL-2) and Interferon-γ (IFN-γ) in Identifying Severe Community-Acquired Pneumonia (SCAP) Clinical Outcomes and Complications. 2011 Eur Resp J 38:1474.

Maleki, K.T. et al. Serum Markers Associated with Severity and Outcome of Hantavirus Pulmonary Syndrome. 2019 J Infect Dis 219:1832-40.

McElroy, A.K. et al. Ebola Hemorrhagic Fever: Novel Biomarker Correlates of Clinical Outcome. 2014 J Infect Dis 210:558-66.

McElroy, A.K. et al. Human Ebola Virus Infection Results in Substantial Immune Activation. 2014 Proc Natl Acad Sci 112:4719-24.

McInnes, I.B. et al. Interleukin-15 Mediates T Cell-Dependent Regulation of Tumor Necrosis Factor-α Production in Rheumatoid Arthritis. 1997 Nat Med 3:189-95.

McKinstry, K.K. et al. Memory CD4 T Cell-Derived IL-2 Synergizes with Viral Infection to Exacerbate Lung Inflammation. 2019 PLoS Pathog 15:e1007989.

Mehta, P. et al. COVID-19: Consider Cytokine Storm Syndromes and Immunosuppression. 2020 Lancet 395:1033-4.

Miyagawa, F., Tagaya, Y., Kim, B.S., Patel, H.J., Ishida, K., Ohteki, T., Waldmann, T.A., Katz, S.I., IL-15 serves as a costimulator in determining the activity of autoreactive CD8 T cells in an experimental mouse model of graft-versus-host-like disease. 2008 J Immunol 181:1109-19.

Mo, X.Y. et al. Induction of Cytokines in Mice with Parainfluenza Pneumonia. 1995 J Virol 69:1288-91.

Moretti, S. et al. A Mast Cell-ILC2-Th9 Pathway Promotes Lung Inflammation in Cystic Fibrosis. 2017 Nat Commun 8:14017.

Mori, M. et al. High Levels of Cytokine-Producing Cells in the Lung Tissues of Patients with Fatal Hantavirus Pulmonary Syndrome. 1999 J Infect Dis 179:295-302.

Muro, S. et al. Expression of IL-15 in Inflammatory Pulmonary Diseases. 2001 J Allergy Clin Immunol 108:970-5.

Nakamura, R. et al. Interleukin-15 Is Critical in the Pathogenesis of Influenza A Virus-Induced Acute Lung Injury. 2010 J Virol 84:5574-82.

Needleman, B.W. et al. Interleukin-1, interleukin-2, interleukin-4, interleukin-6, tumor necrosis factor alpha, and interferon-gamma levels in sera from patients with scleroderma. 1992 Arthritis Rheum 35:67-72.

Noguchi, M., Yi, H., Rosenblatt, H.M., Filipovich, A.H., Adelstein, S., Modi, W.S., McBride, O.W., Leonard, W.J., Interleukin 2 receptor gamma chain mutation results in X-linked severe combined immunodeficiency in humans. 1993 Cell 73:147-57.

Nordberg, M. et al. Cytotoxic mechanisms may play a role in the local immune response in the central nervous system in neuroborreliosis. 2011 J Neuroimmunol 232:186-93.

Notarnicola, A. et al. Correlation between serum levels of IL-15 and IL-17 in patients with idiopathic inflammatory myopathies. 2015 Scand J Rheumatol 44:224-8.

Oh, U., Jacobson S., Treatment of HTLV-I-Associated Myelopathy / Tropical Spastic Paraparesis: Towards Rational Targeted Therapy 2008 Neurol Clin 2008 26: 781-5.

Okamoto, M. et al. Interleukin 18 (IL-18) in Synergy with IL-2 Induces Lethal Lung Injury in Mice: a Potential Role for Cytokines, Chemokines, and Natural Killer Cells in the Pathogenesis of Interstitial Pneumonia. 2002 Blood 99:1289-98.

Olcott, C.M. et al. Interleukin-9 and interleukin-17C in chronic rhinosinusitis. 2016 Int Forum Allergy Rhinol 6:841-7.

Orucevic, A. et al. Role of nitric oxide in IL-2 therapy-induced capillary leak syndrome. 1998 Cancer Metastasis Rev 17:127-42.

Orzaez, M., Gortat, A., Mondragon, L., Perez-Paya, E., Peptides and Peptide Mimics as Modulators of Apototic Pathways. 2009 Chem Med Chem 4:146-60.

O’Shea, J.J., Targeting the Jak/STAT pathway for immunosuppression. 2004 Ann Rheum Dis 63:(suppl II): ii67-71.

Outinen, T.K. et al. Thrombocytopenia Associates with the Severity of Inflammation and Variables Reflecting Capillary Leakage in Puumala Hantavirus Infection, an Analysis of 546 Finnish Patients 2016 Infect Dis (Lond) 48:682-7.

Ozsurekci, Y. et al. Can the Mild Clinical Course of Crimean-Congo Hemorrhagic Fever in Children Be Explained by Cytokine Responses? 2013 J Med Virol 85:1955-9.

Panupattanapong, S. et al. New spectrum of COVID-19 manifestations in children: Kawasaki-like syndrome and hyperinflammatory response. 2020 Cleve Clin J Med in press.

Papa, A. et al. Emergence of Crimean-Congo Haemorrhagic Fever in Greece. 2009 Clin Microbiology and Infection 16:843-7.

Papa, A. et al. Cytokines as Biomarkers of Crimean-Congo Hemorrhagic Fever. 2015 J Med Virol 88:21-7.

Parsonnet, J. et al. Mediators in the Pathogenesis of Toxic Shock Syndrome: Overview. 1989 Rev Infect Dis 11:S263-9.

Patro, A.R.K. et al. Cytokine Signature Associated with Disease Severity in Dengue. 2019 Viruses 88:34;v110100034.

Pattanaik, D. et al. Pathogenesis of Systemic Sclerosis. 2015 Front Immunol 6:272.

Paul, W.E., Pleiotropy and redundancy: T cell-derived lymphokines in the immune response. 1989 Cell 57:521-4.

Pesu M, Candotti F, Husa M, Hofmann SR, Notarangelo LD, and O’Shea JJ. Jak3, severe combined immunodeficiency, and a new class of immunosuppressive drugs. 2005 Immunol Rev 203:127-42.

Pesu, M., Laurence, A., Kishore, N., Zwillich, S., Chan, G., O’Shea, J.J., Therapeutic targeting of Janus kinases. 2008 Immunol Rev 223:132-42.

Pietikainen, A. et al. Cerebrospinal fluid cytokines in Lyme neuroborreliosis. 2016 J Neuroinflammation 13:273.

Poust, J.C. et al. Management of Toxicities Associated with High-Dose Interleukin-2 and Biochemotherapy. 2013 Anticancer Drugs 24:1-13.

Prasse, A. et al. Th1 Cytokine Pattern in Sarcoidosis Is Expressed by Bronchoalveolar CD4 and CD8 T Cells. 2000 Clin Exp Immunol 122:241-8.

Prior, C. et al. Increased Levels of Serum Interferon-Gamma in Pulmonary Sarcoidosis and Relationship with Response to Corticosteroid Therapy. 1991 Am Rev Respir Dis 143:53-60.

Rafi, A.Q. et al. Evidence for the Involvement of Fas Ligand and Perforin in the Induction of Vascular Leak Syndrome. 1998 J Immunol 161:3077-86.

Rai, G. et al. Serum Cytokine Profile in Patients with Chronic Rhinosinusitis with Nasal Polyposis Infected by Aspergillus flavus. 2018 Ann Lab Med 38:125-31.

Ramos-Casals, M. et al. Adult Haemophagocytic Syndrome. 2014 Lancet 383:1503-16.

Rauer, S. et al. Lyme Neuroborreliosis. 2018 Dtsch Arztebl Int 115:751-6.

Robinson, B.W. et al. Gamma Interferon Is Spontaneously Released by Alveolar Macrophages and Lung T Lymphocytes in Patients with Pulmonary Sarcoidosis. 1985 J Clin Invest 75:1488-95.

Rochman, Y., Spolski, R., Leonard, W.J., New Insights into the regulation of T cells by gamma c family cytokines. 2009 Nat Rev Immunol 9:480-90.

Roediger, B. et al. IL-2 Is a Critical Regulator of Group 2 Innate Lymphoid Cell Function during Pulmonary Inflammation. 2015 J Allergy Clin Immunol 136:1653-63.

Ruiz, S.I. et al. Animal Models of Human Viral Diseases. Animal Models for the Study of Human Disease, 2013. 927-70.

Ruprecht, C.R. et al. Coexpression of CD25 and CD27 Identifies FoxP3 Regulatory T Cells in Inflamed Synovia. 2005 J Exp Med 201:1793-1803.

Russier, M. et al. The Exonuclease Domain of Lassa Virus Nucleoprotein Is Involved in Antigen-Presenting-Cell-Mediated NK Cell Responses. 2014 J Virol 88:13811-20.

Sadeghi, M. et al. Cytokine Expression during Early and Late Phase of Acute Puumala Hantavirus Infection. 2011 BMC Immunol 12:65.

Sakaguchi, S., Yamaguchi, T., Nomura, T., Ono, M., Regulatory T cells and immune tolerance. 2008 Cell 133:775-87.

Sambatakou, H. et al. Cytokine Profiling of Pulmonary Aspergillosis. 2006 Int J Immunogenet 33:297-302.

Sarawar, S.R. et al. Cytokine Profiles of Bronchoalveolar Lavage Cells from Mice with Influenza Pneumonia: Consequences of CD4+ and CD8+ T Cell Depletion. 1993 Reg Immunol 5:142-50.

Sarawar, S.R. et al. Concurrent Production of Interleukin-2, Interleukin-10, and Gamma Interferon in the Regional Lymph Nodes of Mice with Influenza Pneumonia. 1994 J Virol 68:3112-9.

Sato, N., Sabzevari, H., Fu, S., Ju, W., Bamford, R.N., Waldmann, T.A., and Tagaya, Y., Development of an IL-15-Autocrine CD8 T-cell Leukemia in IL-15 Transgenic mice requires the cis-expression of IL-15R alpha. 2011 Blood 117:4032-40.

Schaeffer, J. et al. Lassa Virus Activates Myeloid Dendritic Cells but Suppresses Their Ability to Stimulate T Cells. 2018 PLoS Pathog 14:e1007430.

Schaeffer, J. et al. Non-Pathogenic Mopeia Virus Induces More Robust Activation of Plasmacytoid Dendritic Cells than Lassa Virus. 2019 Viruses 11:287.

Schlosser, R.J. et al. Mucous Cytokine Levels in Chronic Rhinosinusitis-Associated Olfactory Loss. 2016 JAMA Otolaryngol Head Neck Surg 142:731-7.

Schulert, G.S. et al. Macrophage Activation Syndrome and Cytokine-Directed Therapies. 2014 Best Pract Res Clin Rheumatol 28:277-92.

Segawa, S. et al. Inhibition of Transforming Growth Factor-β Signalling Attenuates Interleukin (IL)-18 plus IL-2-Induced Interstitial Lung Disease in Mice. 2010 Clin Exp Immunol 160:394-402.

Semenzato, G. et al. Immune Mechanisms in Interstitial Lung Diseases. 2000 Allergy 55:1103-20.

Shaw, T.Y. et al. Weathering a Cytokine Storm: A Case of EBV-Induced Hemophagocytic Lymphohistiocytosis. 2016 J Investig Med High Impact Case Rep 4:2324709616647409.

Shimbara, A. et al. IL-9 and Its Receptor in Allergic and Nonallergic Lung Disease: Increased Expression in Asthma. 2000 J Allergy Clin Immunol 105:108-15.

Shultz, L.D., Brehm, M.A., Garcia-Martinez, J.V., Greiner, D.L., Humanized mice for immune system investigation: progress, promise and challenges. 2012 Nat Rev Immunol 12:786-98.

Silversides, J.A. et al. Staphylococcal Toxic Shock Syndrome: Mechanisms and Management. 2010 Curr Infect Dis Rep 12:392-400.

Singer, M. et al. The Third International Consensus Definitions for Sepsis and Septic Shock (Sepsis-3). 2016 JAMA 315:801-10.

Sisto, M. et al. Interleukin-15 as a Potential New Target in Sjögrens Syndrome-Associated Inflammation. 2016 Pathology 48:602-7.

Sisto, M. et al. TLR2 Signals via NF-KB to Drive IL-15 Production in Salivary Gland Epithelial Cells Derived from Patients with Primary Sjögren’s Syndrome. 2017 Clin Exp Med 17:341-50.

Smith, D.R. et al. Persistent Crimean-Congo Hemorrhagic Fever Virus Infection in the Testes and within Granulomas of Non-Human Primates with Latent Tuberculosis. 2019 PLoS Pathogens 15:e1008050.

Smith, N.L.D. et al. A Prominent Role for the IL1 Pathway and IL15 in Susceptibility to Chronic Cavitary Pulmonary Aspergillosis. 2014 Clin Microbiol Infect 20:0480-8.

Smith, N.L.D. et al. Clinical Implications of Interferon-γ Genetic and Epigenetic Variants. 2014 Immunology 143:499-511.

Smolewska, E. et al. Regulation of Peripheral Blood and Synovial Fluid Lymphocyte Apoptosis in Juvenile Idiopathic Arthritis. 2004 Scand J Rheumatol 33:7-12.

Soussi-Gounni, A. et al. Role of IL-9 in the Pathophysiology of Allergic Diseases. 2001 J Allergy Clin Immunol 107:575-82.

Stern, R.S. et al. Stevens-Johnson Syndrome and Toxic Epidermal Necrolysis: Associations, Outcomes, and Pathobiology - Thirty Years of Progress but Still Much to be Done. 2017 J Invest Dermatol 137:1004-8.

Streckfus, C. et al. Cytokine Concentrations in Stimulated Whole Saliva among Patients with Primary Sjögrens Syndrome, Secondary Sjögrens Syndrome, and Patients with Primary Sjögrens Syndrome Receiving Varying Doses of Interferon for Symptomatic Treatment of the Condition: a Preliminary Study. 2001 Clin Oral Investig 5:133-5.

Strengell, M. et al. IL-21 in Synergy with IL-15 or IL-18 Enhances IFN-γ Production in Human NK and T Cells. 2003 J Immunol 170:5464-9.

Su, S.C. et al. Interleukin-15 Is Associated with Severity and Mortality in Stevens-Johnson Syndrome/Toxic Epidermal Necrolysis. 2017 J Invest Dermatol 137:1065-73.

Sugamura, K., Asao, H., Kondo, M., Tanaka, N., Ishii, N., Nakamura, M., Takeshita, T., The common gamma-chain for multiple cytokine receptors. 1995 Adv Immunol 59:225-77.

Sugamura, K., Asao, H., Kondo, M., Tanaka, N., Ishii, N., Ohbo, K., Nakamura, M., Takeshita, T., The interleukin-2 receptor gamma chain: its role in the multiple cytokine receptor complexes and T cell development in XSCID. 1996 Annu Rev Immunol 14:179-205.

Sugimoto, N. et al. IL-9 Blockade Suppresses Silica-Induced Lung Inflammation and Fibrosis in Mice. 2019 Am J Respir Cell Mol Biol 60:232-43.

Sullivan, N. et al. Ebola Virus Pathogenesis: Implications for Vaccines and Therapies. 2003 J Virol 77:9733-7.

Suntharalingam, G. et al. Cytokine storm in a phase 1 trial of the anti-CD28 monoclonal antibody TGN1412. 2006 N Engl J Med 355:1018-28.

Tagaya, Y., Burton, J.D., Miyamoto, Y., Waldmann, TA., Identification of a novel receptor/signal transduction pathway for IL-15/T in mast cells. 1996 EMBO J. 15:4928-39.

Takai, K., Sawasaki, T., and Endo. Y. The Wheat-Germ Cell-Free Expression System, 2010 Curr. Pharm. Biotechnol. 11:272-8.

Tanaka, T., et al., A novel monoclonal antibody against murine IL-2 receptor beta-chain. Characterization of receptor expression in normal lymphoid cells and EL-4 cells. 1991 J. Immunol. 147:2222-8.

Takeshita, T., Asao, H., Ohtani, K., Ishii, N., Kumaki, S., Tanaka, N., Manukata, H., Nakamura, M., Sugamura, K., Cloning of the Gamma chain of the Human IL2 receptor. 1992 Science 257:379-82.

Tan, M.C. et al. Acute Myocarditis Following High-Dose Interleukin-2 Treatment. 2016 J Cardiol Cases 15:28-31.

Temann, U.A. et al. IL9 Leads to Airway Inflammation by Inducing IL13 Expression in Airway Epithelial Cells. 2007 Int Immunol 19:1-10.

Temann, U.A. et al. Pulmonary Overexpression of IL-9 Induces Th2 Cytokine Expression, Leading to Immune Pathology. 2002 J Clin Invest 109:29-39.

Thiant, S. et al. Plasma Levels of IL-7 and IL-15 in the First Month after Myeloablative BMT Are Predictive Biomarkers of Both Acute GVHD and Relapse. 2010 Bone Marrow Transplant 45:1546-52.

Tisoncik, J.R., Korth, M.J., Simmons, C.P., Farrar, J., Martin, T.R., Katze, M.G. Into the eye of the cytokine storm. 2012 Microbiol Mol Biol Rev 76:16-32.

Tokman, M.G. et al. The Pathogenesis Of Experimental Toxic Shock Syndrome: The Role Of Interleukin-2 In The Induction Of Hypotension And Release Of Cytokines. 1995 Shock 3:145-51.

Tourkova, I.L. et al. Restoration by IL-15 of MHC Class I Antigen-Processing Machinery in Human Dendritic Cells Inhibited by Tumor-Derived Gangliosides. 2005 J Immunol 175:3045-52.

Trottestam, H. et al. Chemoimmunotherapy for Hemophagocytic Lymphohistiocytosis: Long-Term Results of the HLH-94 Treatment Protocol. 2011 Blood 118:4577-84.

Tsoutsou, P.G. et al. Cytokine Levels in the Sera of Patients with Idiopathic Pulmonary Fibrosis. 2006 Respir Med 100:938-45.

Uchiyama, T. et al. Study of the Biological Activities of Toxic Shock Syndrome Toxin-1. 1986 Microbiol Immunol 30:469-83.

Ueda, T. et al. Serum Interleukin-15 Level Is a Useful Predictor of the Complications and Mortality in Severe Acute Pancreatitis. 2007 Surgery 142:319-26.

van den Brûle, S. et al. Profibrotic Effect of IL-9 Overexpression in a Model of Airway Remodeling. 2007 Am J Respir Cell Mol Biol 37:202-9.

Veenhuis, R.T. et al. Systemic Elevation of Proinflammatory Interleukin 18 in HIV/HCV Coinfection versus HIV or HCV Monoinfection. 2017 Clin Infect Dis 64:589-96.

Via, C.S. et al. Kinetics of T Cell Activation in Acute and Chronic Forms of Murine Graft-versus-Host Disease. 1991 J Immunol 146:2603-9.

Via, C.S. et al. Critical Role of Interleukin-2 in the Development of Acute Graft-versus-Host Disease. 1993 Int Immunol 5:565-72.

Villinger, F. et al. Markedly Elevated Levels of Interferon (IFN)-σ, IFN-α, Interleukin (IL)-2, IL-10, and Tumor Necrosis Factor-α Associated with Fatal Ebola Virus Infection. 1999 J Infect Dis 179:S188-91.

Vissinga, C. et al. TCR Expression and Clonality Analysis in Pulmonary Sarcoidosis. 1996 Hum Immunol 48:98-106.

Waldmann, T.A., Anti-Tac (daclizumab, Zenapax) in the treatment of leukemia, autoimmune diseases, and in the prevention of allograft rejection: a 25-year personal odyssey. 2007 J Clin Immunol 27: 1-18.

Waldmann T.A. et al. The Multifaceted Regulation Of Interleukin-15 Expression And The Role Of This Cytokine In Nk Cell Differentiation And Host Response To Intracellular Pathogens. 1999 Annu Rev Immunol 17:19-49.

Wang, Z. et al. Biomarkers of Cytokine Release Syndrome and Neurotoxicity Related to CAR-T Cell Therapy. 2018 Biomark Res 6:4.

Watanabe, R. et al. Pro-inflammatory and anti-inflammatory T cells in giant cell arteritis. 2017 Joint Bone Spine 84:421-6.

Wauquier, N. et al. Human Fatal Zaire Ebola Virus Infection Is Associated with an Aberrant Innate Immunity and with Massive Lymphocyte Apoptosis. 2010 PLoS Negl Trop Dis 4:e0000837.

Wei, S. et al. Activation of Tumor Necrosis Factor-Alpha Production from Human Neutrophils by IL-2 via IL-2-R Beta. 1993 J Immunol 150:1979-87.

Welbourn, R. et al. Involvement of Thromboxane and Neutrophils in Multiple-System Organ Edema with Interleukin-2. 1990 Ann Surg 212:728-33.

Welbourn, R. et al. Interleukin-2 Induces Early Multisystem Organ Edema Mediated by Neutrophils. 1991 Ann Surg 214:181-6.

Welch, S.R. et al. Fluorescent Crimean-Congo Hemorrhagic Fever Virus Illuminates Tissue Tropism Patterns and Identifies Early Mononuclear Phagocytic Cell Targets in Ifnar-/- Mice. 2019 PLoS Pathog 15:e1008183.

Weyand, C.M. et al. Disease patterns and tissue cytokine profiles in giant cell arteritis. 1997 Arthritis Rheum 40:19-26.

White, R.L. et al. Cardiopulmonary Toxicity of Treatment with High Dose Interleukin-2 in 199 Consecutive Patients with Metastatic Melanoma or Renal Cell Carcinoma. 1994 Cancer 74:3212-22.

Williams, A.E. et al. The Mercurial Nature of Neutrophils: Still an Enigma in ARDS? 2014 Am J Physiol Lung Cell Mol Physiol 306:L217-30.

Winn, R.M. et al. Selective Effects of Interleukin (IL)-15 on Antifungal Activity and IL-8 Release by Polymorphonuclear Leukocytes in Response to Hyphae of Aspergillus Species. 2003 J Infect Dis 188:585-90.

Wuttge, D.M. et al. Serum IL-15 in patients with early systemic sclerosis: a potential novel marker of lung disease. 2007 Arthritis Res Ther 9:R85.

Xie, Z. et al. Inflammatory Markers of the Systemic Capillary Leak Syndrome (Clarkson Disease). 2014 J Clin Cell Immunol 5:1000213.

Xu, W. et al. IL-9 Blockade Attenuates Inflammation in a Murine Model of Methicillin-Resistant Staphylococcus Aureus Pneumonia. 2020 Acta Biochim Biophys Sin 52:133-40.

Yang, J. et al. Interleukin-2 and Lymphocyte-Induced Eosinophil Proliferation and Survival in Asthmatic Patients. 1993 J Allergy Clin Immunol 91:792-801.

Yang, L. et al. Epstein-Barr virus (EBV)-encoded RNA promotes growth of EBV-infected T cells through interleukin-9 induction. 2004 Cancer Res 64:5332-7.

Yang, Y. et al. TCR Engagement Negatively Affects CD8 but Not CD4 CAR T Cell Expansion and Leukemic Clearance. 2017 Sci Transl Med 9:eaag1209.

Yarkoni, S. et al. IL-2-targeted therapy ameliorates the severity of graft-versus-host disease: ex vivo selective depletion of host-reactive T cells and in vivo therapy. 2012 Biol Blood Marrow Transplant 18:523-35.

Youinou, P. et al. Disturbance of Cytokine Networks in Sjögrens Syndrome. 2011 Arthritis Res Ther 13:227.

Younan, P. et al. Ebola Virus Binding to Tim-1 on T Lymphocytes Induces a Cytokine Storm. 2017 MBio 8:00847-17.

Younan, P. et al. Ebola Virus-Mediated T-Lymphocyte Depletion Is the Result of an Abortive Infection. 2019 PLoS Pathog 15:e1008068.

Yuki, K. et al. COVID-19 pathophysiology: A review. 2020 Clin Immunol 215:108427.

Zhang, X. et al. Potent and Selective Stimulation of Memory-Phenotype CD8 T Cells In Vivo by IL-15. 1998 Immunity 8:591-9.

Zhou, Y. et al. Th2 cytokines and asthma. Interleukin-9 as a therapeutic target for asthma. 2001 Respir Res 2:80-4.

Zinter, M.S. et al. Calming the Storm in HLH. 2019 Blood 134:103-4.

What is claimed is:

1. A composition, comprising:
an effective amount of a therapeutic compound, wherein the effective amount of the therapeutic compound is an amount sufficient to modulate signaling by at least one IL-2, IL-9, and IL-15 γc-cytokine family member and to thereby inhibit, ameliorate, reduce a severity of, treat, delay the onset of, or prevent at least one cytokine storm related disorder; and
a pharmaceutically acceptable carrier.

2. The composition of claim 1, wherein the at least one cytokine storm related disorder is selected from the group consisting of cytokine release syndrome, multiple organ dysfunction syndrome, systemic inflammatory response syndrome, sepsis, septic shock, graft-versus-host disease, haploidentical donor transplantation, sarcoidosis, hemophagocytic lymphohistiocytosis, vascular leak syndrome, Stevens-Johnson syndrome, toxic epidermal necrolysis, asthmatic allergic lung inflammation, rhinosinusitis, viral infection, multi-system inflammatory syndrome in children (MIS-C) associated with COVID-19, viral hemorrhagic fever, HIV/HCV coinfection liver fibrosis, fungal infection, pulmonary Aspergillosis, bacterial infection, toxic shock syndrome, lyme neuroborreliosis, autoimmune disease, macrophage activation syndrome, T-cell based immunotherapy, immune effector cell-associated neurotoxicity syndrome, and pulmonary infiltrate.

3. The composition of claim 2, wherein the viral infection is due to one or more of coronavirus, influenza virus, Lass virus, Rift Valley fever virus, Crimean-Congo hemorrhagic fever virus, Yellow Fever virus, Dengue Fever virus, Ebola virus, Marburg virus, hantavirus, and Epstein-Barr virus.

4. The composition of claim 3, wherein the coronavirus is one or more of SARS-CoV-1, SARS-CoV-2, and MERS-CoV.

5. The composition of claim 2, wherein the autoimmune disease is due to one or more of juvenile idiopathic arthritis, Still’s disease, Sjögren’s syndrome, systemic sclerosis, inflammatory myopathies, systemic vasculitides, giant cell arteritis, Horton disease, cranial arteritis, and temporal arteritis.

6. The composition of claim 2, wherein the T-cell based immunotherapy is one or more of chimeric antigen receptor T-cell therapy and T-cell bispecific antibody therapy.

7. The composition of claim 2, wherein the pulmonary infiltrate is due to one or more of adult respiratory distress syndrome, interstitial lung disease, pneumonia, community acquired pneumonia, and acute interstitial pneumonia.

8. The composition of claim 1, wherein the therapeutic compound is at least one of a γc-cytokine antagonist peptide, a γc-cytokine antagonist peptide derivative, or a combination thereof.

9. The composition of claim 8, wherein the γc-cytokine antagonist peptide comprises a partial sequence of a γc-box D-helix region of each of at least IL-2 and IL-15 γc-cytokine family members.

10. The composition of claim 9, wherein the partial sequence comprises consecutive blocks of at least 5 amino acids of the γc-box D-helix region of each of at least IL-2 and IL-15 γc-cytokine family members.

11. The composition of claim 9, wherein the partial sequence comprises consecutive blocks of 1-10 amino acids of the γc-box D-helix region of each of at least IL-2 and IL-15 γc-cytokine family members.

12. The composition of any one of claims 8-11, wherein the γc-cytokine antagonist peptide comprises 11 to 50 amino acids.

13. The composition of any one of claims 8-12, wherein the γc-cytokine antagonist peptide further comprises a conjugate at the N-termini, C-termini, side residues, or a combination thereof.

14. The compositions of claim 13, wherein the conjugate comprises one or more additional moieties selected from the group consisting of bovine serum albumin (BSA), albumin, Keyhole Limpet Hemocyanin (KLH), Fc region of IgG, a biological protein that functions as scaffold, an antibody against a cell-specific antigen, a receptor, a ligand, a metal ion, and Poly Ethylene Glycol (PEG).

15. The composition of any one of claims 8-14, wherein the γc-cytokine antagonist peptide further comprises a signal peptide.

16. The composition of any one of claims 8-15, wherein the γc-cytokine antagonist peptide comprises a sequence of SEQ ID NO: 1 (BNZ-γ).

17. The composition of any one of claims 8-15, wherein the γc-cytokine antagonist peptide consists of a sequence of SEQ ID NO: 1.

18. The composition of claim 8, wherein the γc-cytokine antagonist peptide and the γc-antagonist peptide derivative have similar physico-chemical properties but distinct IL-2, IL-9, or IL-15 biological activities.

19. The composition of claim 8, wherein the γc-cytokine antagonist peptide derivative shares at least about 60% identity with a peptide of SEQ ID NO: 1.

20. The composition of claim 8, wherein the γc-cytokine antagonist peptide derivative shares at least about 90% identity with a peptide of SEQ ID NO: 1.

21. The composition of claim 8, wherein the γc-cytokine antagonist peptide derivative shares at least about 95% identity with a peptide of SEQ ID NO: 1.

22. The composition of any one of claims 1-21, wherein the pharmaceutically acceptable carrier is formulated for topical, oral, and/or parenteral delivery.

23. The composition of any one of claims 1-21, wherein the pharmaceutically acceptable carrier is formulated for topical delivery.

24. The composition of any one of claims 1-21, wherein the pharmaceutically acceptable carrier is formulated for oral delivery.

25. The composition of any one of claims 1-21, wherein the pharmaceutically acceptable carrier is formulated for parenteral delivery.

26. A method of inhibiting, ameliorating, reducing a severity of, treating, delaying the onset of, or preventing at least one cytokine storm related disorder, the method comprising:
administering the composition of any one of claims 1-25 to a subject in need thereof,
thereby inhibiting, ameliorating, reducing a severity of, treating, delaying the onset of, or preventing the at least one cytokine storm related disorder.

27. The method of claim 26, wherein the at least one cytokine storm related disorder is selected from the group consisting of cytokine release syndrome, multiple organ dysfunction syndrome, systemic inflammatory response syndrome, sepsis, septic shock, graft-versus-host disease, haploidentical donor transplantation, sarcoidosis, hemophagocytic lymphohistiocytosis, vascular leak syndrome, Stevens-Johnson syndrome, toxic epidermal necrolysis, asthmatic allergic lung inflammation, rhinosinusitis, viral infection, multi-system inflammatory syndrome in children (MIS-C) associated with COVID-19, viral hemorrhagic fever, HIV/HCV coinfection liver fibrosis, fungal infection, pulmonary Aspergillosis, bacterial infection, toxic shock syndrome, lyme neuroborreliosis, autoimmune disease, macrophage activation syndrome, T-cell based immunotherapy, immune effector cell-associated neurotoxicity syndrome, and pulmonary infiltrate.

28. The method of claim 27, wherein the viral infection is due to one or more of coronavirus, influenza virus, Lass virus, Rift Valley fever virus, Crimean-Congo hemorrhagic fever virus, Yellow Fever virus, Dengue Fever virus, Ebola virus, Marburg virus, hantavirus, and Epstein-Barr virus.

29. The method of claim 28, wherein the coronavirus is one or more of SARS-CoV-1, SARS-CoV-2, and MERS-CoV.

30. The method of claim 27, wherein the autoimmune disease is due to one or more of juvenile idiopathic arthritis, Still’s disease, Sjögren’s syndrome, systemic sclerosis, inflammatory myopathies, systemic vasculitides, giant cell arteritis, Horton disease, cranial arteritis, and temporal arteritis.

31. The method of claim 27, wherein the T-cell based immunotherapy is one or more of chimeric antigen receptor T-cell therapy and T-cell bispecific antibody therapy.

32. The method of claim 27, wherein the pulmonary infiltrate is due to one or more of adult respiratory distress syndrome, interstitial lung disease, pneumonia, community acquired pneumonia, and acute interstitial pneumonia.

33. A method of designing a γc-cytokine antagonist peptide and/or a derivative thereof configured to modulate and/or block signaling by at least one IL-2, IL-9, and IL-15 γc-cytokine family member that inhibits, ameliorates, reduces a severity of, treats, delays the onset of, or prevents at least one cytokine storm related disorder, the method comprising the steps of:
using a computer to obtain from an amino acid sequence database amino acid sequences of at least one IL-2 and IL-15 γc-cytokine family member,
assembling a γc-cytokine antagonist peptide and/or a derivative thereof based on a sequence of the at least one IL-2 and IL-15 γc-cytokine family member,
wherein the γc-cytokine antagonist peptide and/or the derivative thereof modulates and/or blocks signaling by the at least one IL-2, IL-9, and IL-15 γc-cytokine family member.

34. The method of claim 33, wherein the γc-cytokine antagonist peptide comprises a partial sequence of a γc-box D-helix region of each of at least two IL-2 and IL-15 γc-cytokine family members.

35. The method of claim 34, wherein the partial sequence comprises consecutive blocks of at least 5 amino acids of the γc-box D-helix region of each of at least two IL-2 and IL-15 γc-cytokine family members.

36. The method of claim 34, wherein the partial sequence comprises consecutive blocks of 1-10 amino acids of the γc-box D-helix region of each of at least two IL-2 and IL-15 γc-cytokine family members.

37. The method of any one of claims 33-36, wherein the γc-cytokine antagonist peptide comprises 11 to 50 amino acids.

38. The method of any one of claims 33-37, wherein the γc-cytokine antagonist peptide further comprises a conjugate at the N-termini, C-termini, side residues, or a combination thereof.

39. The method of any one of claims 33-38, wherein the γc-cytokine antagonist peptide further comprises a signal peptide.

40. The method of any one of claims 33-39, wherein the γc-cytokine antagonist peptide and the derivative thereof have similar physico-chemical properties but distinct IL-2, IL-9, and IL-15 biological activities.

41. The method of any one of claims 33-39, wherein the γc-cytokine antagonist peptide comprises a sequence of SEQ ID NO: 1 (BNZ-y).

42. The method of any one of claims 33-39, wherein the γc-cytokine antagonist peptide consists of a sequence of SEQ ID NO: 1.

43. The method of any one of claims 33-40, wherein the γc-cytokine antagonist peptide derivative shares at least about 60% identity with a peptide of SEQ ID NO: 1.

44. The method of any one of claims 33-40, wherein the γc-cytokine antagonist peptide derivative shares at least about 90% identity with a peptide of SEQ ID NO: 1.

45. The method of any one of claims 33-40, wherein the γc-cytokine antagonist peptide derivative shares at least about 95% identity with a peptide of SEQ ID NO: 1.

46. A kit for inhibiting, ameliorating, reducing a severity of, treating, delaying the onset of, or preventing at least one cytokine storm related disorder comprising:
a composition according to any one of claims 1-25.

47. The kit of claim 46, wherein the at least one cytokine storm related disorder is selected from the group consisting of cytokine release syndrome, multiple organ dysfunction syndrome, systemic inflammatory response syndrome, sepsis, septic shock, graft-versus-host disease, haploidentical donor transplantation, sarcoidosis, hemophagocytic lymphohistiocytosis, vascular leak syndrome, Stevens-Johnson syndrome, toxic epidermal necrolysis, asthmatic allergic lung inflammation, rhinosinusitis, coronavirus, SARS-CoV-1, SARS-CoV-2, MERS-CoV, influenza virus, Lass virus, Rift Valley fever virus, Crimean-Congo hemorrhagic fever virus, Yellow Fever virus, Dengue Fever virus, Ebola virus, Marburg virus, hantavirus, and Epstein-Barr virus, multi-system inflammatory syndrome in children (MIS-C) associated with COVID-19, viral hemorrhagic fever, HIV/HCV coinfection liver fibrosis, fungal infection, pulmonary Aspergillosis, bacterial infection, toxic shock syndrome, lyme neuroborreliosis, juvenile idiopathic arthritis, Still’s disease, Sjögren’s syndrome, systemic sclerosis, inflammatory myopathies, systemic vasculitides, giant cell arteritis, Horton disease, cranial arteritis, temporal arteritis, macrophage activation syndrome, T-cell based immunotherapy, chimeric antigen receptor T-cell therapy, T-cell bispecific antibody therapy, immune effector cell-associated neurotoxicity syndrome, and pulmonary infiltrate, adult respiratory distress syndrome, interstitial lung disease, pneumonia, community acquired pneumonia, and acute interstitial pneumonia.


SEQUENCE LISTING (23 sequences)

SEQ ID NO: 1 - protein, 19 aa, Artificial
  Synthetic
IKEFLQRFIHIVQSIINTS

SEQ ID NO: 2 - protein, 19 aa, Artificial
  Synthetic
IKEFLQSFVHIVQMFINTS

SEQ ID NO: 3 - protein, 20 aa, Artificial
  Synthetic
IVEFLNRWITFCQSIISTLT

SEQ ID NO: 4 - protein, 21 aa, Artificial
  Synthetic
PKEFLERFKSLLQKMIHQHLS

SEQ ID NO: 5 - protein, 21 aa, Artificial
  Synthetic
LENFLERLKTIMREKYSKCSS

SEQ ID NO: 6 - protein, 21 aa, Artificial [sequence omitted]

SEQ ID NO: 7 - protein, 20 aa, Artificial
  Synthetic
DLCFLKRLLQEIKTCWNKIL

SEQ ID NO: 8 - protein, 19 aa, Artificial
  Synthetic
  MISC_FEATURE (1)..(1): X = Any amino acid
  MISC_FEATURE (2)..(2): X = Any amino acid
  MISC_FEATURE (3)..(3): X = D or E
  MISC_FEATURE (6)..(6): X = E or Q or N or polar amino acid
  MISC_FEATURE (7)..(7): X = S or R or polar amino acid
  MISC_FEATURE (8)..(8): X = Non-polar amino acid
  MISC_FEATURE (9)..(9): X = I or K or non-polar amino acid
  MISC_FEATURE (10)..(10): X = Any amino acid
  MISC_FEATURE (11)..(11): X = L or I or aliphatic amino acid
  MISC_FEATURE (12)..(12): X = Non-polar amino acid
  MISC_FEATURE (14)..(14): X = Charged amino acid
  MISC_FEATURE (15)..(15): X = Any amino acid
  MISC_FEATURE (16)..(16): X = I or K
  MISC_FEATURE (17)..(17): X = Any amino acid
  MISC_FEATURE (18)..(18): X = Any amino acid
  MISC_FEATURE (19)..(19): X = Any amino acid
XXXFLXXXXXXXQXXXXXX

SEQ ID NO: 9 - protein, 19 aa, Artificial
  Synthetic
  MISC_FEATURE (1)..(1): X = Any amino acid
  MISC_FEATURE (2)..(2): X = Any amino acid
  MISC_FEATURE (3)..(3): X = D or E
  MISC_FEATURE (6)..(6): X = E or Q or N or polar amino acid
  MISC_FEATURE (7)..(7): X = S or R or polar amino acid
  MISC_FEATURE (8)..(8): X = Non-polar amino acid
  MISC_FEATURE (9)..(9): X = I or K or non-polar amino acid
  MISC_FEATURE (10)..(10): X = Any amino acid
  MISC_FEATURE (11)..(11): X = L or I or aliphatic amino acid
  MISC_FEATURE (12)..(12): X = Non-polar amino acid
  MISC_FEATURE (14)..(14): X = Charged amino acid
  MISC_FEATURE (15)..(15): X = Any amino acid
  MISC_FEATURE (17)..(17): X = Any amino acid
XXXFLXXXXXXXQXXIXTS

SEQ ID NO: 10 - protein, 10 aa, Artificial
  Synthetic
  MISC_FEATURE (1)..(1): X = Any amino acid
  MISC_FEATURE (2)..(2): X = Any amino acid
  MISC_FEATURE (3)..(3): X = Any amino acid
  MISC_FEATURE (4)..(4): S-pentenylalanine; Linked to S-pentenylalanine at position 8
  MISC_FEATURE (5)..(5): X = Any amino acid
  MISC_FEATURE (6)..(6): X = Any amino acid
  MISC_FEATURE (7)..(7): X = Any amino acid
  MISC_FEATURE (8)..(8): S-pentenylalanine; Linked to S-pentenylalanine at position 4
  MISC_FEATURE (9)..(9): X = Any amino acid
  MISC_FEATURE (10)..(10): X = Any amino acid
XXXAXXXAXX

SEQ ID NO: 11 - protein, 11 aa, Artificial
  Synthetic
  MISC_FEATURE (1)..(1): X = Any amino acid
  MISC_FEATURE (2)..(2): X = Any amino acid
  MISC_FEATURE (3)..(3): R-octenylalanine; Linked to S-pentenylalanine at position 10
  MISC_FEATURE (4)..(4): X = Any amino acid
  MISC_FEATURE (5)..(5): X = Any amino acid
  MISC_FEATURE (6)..(6): X = Any amino acid
  MISC_FEATURE (7)..(7): X = Any amino acid
  MISC_FEATURE (8)..(8): X = Any amino acid
  MISC_FEATURE (9)..(9): X = Any amino acid
  MISC_FEATURE (10)..(10): S-pentenylalanine; Linked to R-octenylalanine at position 3
  MISC_FEATURE (11)..(11): X = Any amino acid
XXAXXXXXXAX

SEQ ID NO: 12 - protein, 20 aa, Artificial
  Synthetic
  MISC_FEATURE (1)..(1): S-pentenylalanine; Linked to S-pentenylalanine at position 5
  MISC_FEATURE (5)..(5): S-pentenylalanine; Linked to S-pentenylalanine at position 1
AIKEALQRFIHIVQSIINTS

SEQ ID NO: 13 - protein, 19 aa, Artificial
  Synthetic
  MISC_FEATURE (8)..(8): S-pentenylalanine; Linked to S-pentenylalanine at position 12
  MISC_FEATURE (12)..(12): S-pentenylalanine; Linked to S-pentenylalanine at position 8
IKEFLQRAIHIAQSIINTS

SEQ ID NO: 14 - protein, 19 aa, Artificial
  Synthetic
  MISC_FEATURE (8)..(8): R-octenylalanine; Linked to S-pentenylalanine at position 15
  MISC_FEATURE (15)..(15): S-pentenylalanine; Linked to R-octenylalanine at position 8
IKEFLQRAIHIVQSAINTS

SEQ ID NO: 15 - protein, 19 aa, Artificial
  Synthetic
  MISC_FEATURE (12)..(12): S-pentenylalanine; Linked to S-pentenylalanine at position 16
  MISC_FEATURE (16)..(16): S-pentenylalanine; Linked to S-pentenylalanine at position 12
IKEFLQRFIHIAQSIANTS

SEQ ID NO: 16 - protein, 19 aa, Artificial
  Synthetic
  MISC_FEATURE (12)..(12): R-octenylalanine; Linked to S-pentenylalanine at position 19
  MISC_FEATURE (19)..(19): S-pentenylalanine; Linked to R-octenylalanine at position 12
IKEFLQRFIHIAQSIINTA

SEQ ID NO: 17 - protein, 20 aa, Artificial
  Synthetic
  MISC_FEATURE (1)..(1): S-pentenylalanine; Linked to S-pentenylalanine at position 5
  MISC_FEATURE (5)..(5): S-pentenylalanine; Linked to S-pentenylalanine at position 1
  MISC_FEATURE (9)..(9): S-pentenylalanine; Linked to S-pentenylalanine at position 13
  MISC_FEATURE (13)..(13): S-pentenylalanine; Linked to S-pentenylalanine at position 9
AIKEALQRAIHIAQSIINTS

SEQ ID NO: 18 - protein, 20 aa, Artificial
  Synthetic
  MISC_FEATURE (1)..(1): S-pentenylalanine; Linked to S-pentenylalanine at position 5
  MISC_FEATURE (5)..(5): S-pentenylalanine; Linked to S-pentenylalanine at position 1
  MISC_FEATURE (9)..(9): R-octenylalanine; Linked to S-pentenylalanine at position 16
  MISC_FEATURE (16)..(16): S-pentenylalanine; Linked to R-octenylalanine at position 8
AIKEALQRAIHIVQSAINTS

SEQ ID NO: 19 - protein, 20 aa, Artificial
  Synthetic
  MISC_FEATURE (1)..(1): S-pentenylalanine; Linked to S-pentenylalanine at position 5
  MISC_FEATURE (5)..(5): S-pentenylalanine; Linked to S-pentenylalanine at position 1
  MISC_FEATURE (13)..(13): S-pentenylalanine; Linked to S-pentenylalanine at position 17
  MISC_FEATURE (17)..(17): S-pentenylalanine; Linked to S-pentenylalanine at position 13
AIKEALQRFIHIAQSIANTS

SEQ ID NO: 20 - protein, 20 aa, Artificial
  Synthetic
  MISC_FEATURE (1)..(1): S-pentenylalanine; Linked to S-pentenylalanine at position 5
  MISC_FEATURE (5)..(5): S-pentenylalanine; Linked to S-pentenylalanine at position 1
  MISC_FEATURE (13)..(13): R-octenylalanine; Linked to S-pentenylalanine at position 20
  MISC_FEATURE (20)..(20): S-pentenylalanine; Linked to R-octenylalanine at position 13
AIKEALQRFIHIAQSIINTA

SEQ ID NO: 21 - protein, 20 aa, Artificial
  Synthetic
  MISC_FEATURE (1)..(1): S-pentenylalanine; Linked to S-pentenylalanine at position 5
  MISC_FEATURE (5)..(5): S-pentenylalanine; Linked to S-pentenylalanine at position 1
  MISC_FEATURE (9)..(9): S-pentenylalanine; Linked to S-pentenylalanine at position 13
  MISC_FEATURE (13)..(13): S-pentenylalanine; Linked to S-pentenylalanine at position 9
  MISC_FEATURE (18)..(18): D stereochemical configuration
  MISC_FEATURE (19)..(19): D stereochemical configuration
  MISC_FEATURE (20)..(20): D stereochemical configuration
AIKEALQRAIHIAQSIINTS

SEQ ID NO: 22 - protein, 20 aa, Artificial
  Synthetic
  MISC_FEATURE (1)..(1): S-pentenylalanine; Linked to S-pentenylalanine at position 5
  MISC_FEATURE (5)..(5): S-pentenylalanine; Linked to S-pentenylalanine at position 1
  MISC_FEATURE (9)..(9): R-octenylalanine; Linked to S-pentenylalanine at position 16
  MISC_FEATURE (16)..(16): S-pentenylalanine; Linked to R-octenylalanine at position 9
  MISC_FEATURE (18)..(18): D stereochemical configuration
  MISC_FEATURE (19)..(19): D stereochemical configuration
  MISC_FEATURE (20)..(20): D stereochemical configuration
AIKEALQRAIHIVQSAINTS

SEQ ID NO: 23 - protein, 20 aa, Artificial
  Synthetic
  MISC_FEATURE (1)..(1): S-pentenylalanine; Linked to S-pentenylalanine at position 5
  MISC_FEATURE (5)..(5): S-pentenylalanine; Linked to S-pentenylalanine at position 1
  MISC_FEATURE (13)..(13): S-pentenylalanine; Linked to S-pentenylalanine at position 17
  MISC_FEATURE (17)..(17): S-pentenylalanine; Linked to S-pentenylalanine at position 13
  MISC_FEATURE (18)..(18): D stereochemical configuration
  MISC_FEATURE (19)..(19): D stereochemical configuration
  MISC_FEATURE (20)..(20): D stereochemical configuration
AIKEALQRFIHIAQSIANTS